(19) United States
(12) Patent Application Publication
(10) Pub. No.: US 2024/0043790 A1
(43) Pub. Date: Feb. 8, 2024

(54) LACTIC ACID BACTERIA HAVING IMPROVED SUGAR METABOLISM

(71) Applicant: DUPONT NUTRITION BIOSCIENCES APS, Copenhagen K (DK)

(72) Inventors: Anais JEDRZEJOWSKI, Dange-Saint-Romain (FR); Christophe FREMAUX, Dange-Saint-Romain (FR); Elodie GUILLOU, Dange-Saint-Romain (FR); Sabine VAN DILLEN, Dange-Saint-Romain (FR); Philippe HORVATH, Dange-Saint-Romain (FR); Mikael PIANFETTI, Dange-Saint-Romain (FR)

(21) Appl. No.: 18/050,511

(22) PCT Filed: Apr. 29, 2021
(86) PCT No.: PCT/EP2021/061244

§ 371 Date: Oct. 28, 2022

(30) Foreign Application Priority Data
Apr. 29, 2020 (EP) 20172078.6

(51) Int. Cl. C12N 1/20; C12N 15/74; A23C 19/032; A23C 19/068

(52) U.S. Cl. CPC C12N 1/205; C12N 1/20; C12N 15/746; A23C 19/0323; A23C 19/0684; C12R 2001/46

(57) ABSTRACT

The invention relates to a method for generating a Streptococcus thermophilus strain exhibiting a “high galactose utilization” profile, which is characterized by a high percentage of consumed galactose at the maximum speed of lactose consumption and optionally a high percentage of consumed galactose at the end of lactose consumption (both as determined by assay I), comprising modifying the sequence of the gal-lac gene cluster. The invention also relates to a Streptococcus thermophilus strain obtained or obtainable by this method, to a Streptococcus thermophilus strain characterized both by the sequence of its gal-lac gene cluster and its “high galactose utilization” profile, to a culture and kit-of part comprising such strains, as well as to the use of such strains in food applications.

### CROSS-REFERENCE TO RELATED APPLICATIONS

This specification claims priority under 35 USC § 371 as a national phase of International Patent Application PCT/EP2021/061244 (filed Apr. 29, 2021; and published Nov. 4, 2021 as Int'l Publ. No. WO2021/219777), which, in turn, claims priority to European Patent Application No. 20172078.6, filed Apr. 29, 2020, entitled “LACTIC ACID BACTERIA HAVING IMPROVED SUGAR METABOLISM.” The contents of each of the above-referenced patent applications are incorporated by reference in their entirety into this specification for all purposes.

### INCORPORATION BY REFERENCE OF THE SEQUENCE LISTING

The present application is being filed along with a Sequence Listing in electronic format. The Sequence Listing is provided as a file entitled NB41753-WO-PCT_ST25.txt, created Apr. 26, 2021, which is 57,975 bytes in size. The information in the electronic format of the Sequence Listing is incorporated by reference in its entirety.

### FIELD OF THE INVENTION

The invention relates to a method for generating a Streptococcus thermophilus strain exhibiting a “high galactose utilization” profile, which is characterized by a high percentage of consumed galactose at the maximum speed of lactose consumption and optionally a high percentage of consumed galactose at the end of lactose consumption (both as determined by assay I), comprising modifying the sequence of the gal-lac gene cluster. The invention also relates to a Streptococcus thermophilus strain obtained or obtainable by this method, to a Streptococcus thermophilus strain characterized both by the sequence of its gal-lac gene cluster and its “high galactose utilization” profile, to a culture and kit-of part comprising such strains, as well as to the use of such strains in food applications.

### BACKGROUND TO THE INVENTION

Most strains of the species Streptococcus thermophilus display a galactose-negative phenotype (i.e., unable to grow on galactose as a sole source of carbohydrate). Actually, de Vin et al. (Appl. Environ. Microbiol. 2005) have identified that about 15% of S. thermophilus strains (8 out of 49) are galactose-positive. However, genomic analyses revealed that most if not all S. thermophilus strains possesses an apparently intact galactose operon. The galactose (gal) operon consists in a group of three genes forming the cluster galKTE that is associated to galR and galM genes (FIG. 1) and is followed by the lactose operon that is bearing the lacS and lacZ genes (as described by Vaughan et al. J. Bacteriol. 2001; Vaillancourt et al. J. Bacteriol. 2002). The gal operon and lac operon are defined together as the gal-lac gene cluster. The gal-lac gene cluster organization and location are further defined below. The genes found in the gal operon encode a transcription regulator (galR), a galactokinase (galK), a galactose 1-phosphate uridyltransferase (galT), an UDP-glucose 4-epimerase (galE) and a mutarotase (galM). The enzymes encoded within this operon allows the utilization of galactose through the Leloir pathway (FIG. 2).

It is estimated that the galactose-negative phenotype of S. thermophilus resulted from recent evolution of this species (Vaughan et al, 2001 above cited). All the necessary genetic material being still encoded in their genome, it was thus not surprising to be able to select galactose-positive mutants under appropriate selective conditions as described by multiple authors (Tinson et al., Aust. J. Dairy Technol. 1982; Thomas and Crow, Appl. Environ. Microbiol. 1984; Hutkins et al., Appl. Environ. Microbiol. 1985; Bentaya et al., Can. J. Microbiol. 1991; Vaughan et al., 2001). However, these mutants were unstable, and the galactose-positive phenotype is rapidly lost if selective conditions are not maintained (Thomas and Crow, 1984, above cited), as for example if strains are grown on lactose or on saccharose. Moreover, despite of being galactose-positive, these were still excreting galactose during growth on lactose.

Most of strains are utilizing the galactose moiety of lactose when grown on lactose (Vaillancourt et al. 2002 and de Vin et al. 2005, above cited). In 2005, de Vin et al. have identified 4 type of kinetic of galactose utilization for S. thermophilus, that were defined from type A to type D. Some strains (18.4%) displayed fermentation profile A and consumed none of the excreted galactose within 8.5 h of fermentation. The majority of the strains (65.3%) displayed fermentation profile B, and were only able to consume part of the excreted galactose within 8.5 h of fermentation. Strains belonging to this group consumed the excreted galactose at various speed and to various extent, but never to completion. The inability of the members of group A and B to use all of the excreted galactose was also persistent after prolonged incubation up to 24 h. Other strains (14.3%) displayed a fermentation profile C and consumed all of the excreted galactose within 8.5 h of fermentation.

A single strain (out of 49), with a fermentation profile D, did not excrete galactose during growth on lactose and consumed the glucose and galactose moieties of lactose simultaneously. This strain with the fermentation profile D (called EU20) was part of the Rhodia Food Collection (see Marshall et al. Carbohydrate Research 2001) and later of the DuPont Danisco culture collection (DGCC7698). The DGCC7698 strain was deposited by DuPont Nutrition Biosciences ApS under accession number DSM32823 on May 29, 2018. In 2005, the gal operon of the EU20 strain was registered in Genbank under accession number AY704367.1.

There is still a need to design S. thermophilus strains having an efficient galactose utilization in media containing lactose (such as milk), since in some dairy applications, high quantity of galactose in final products is not desired.

### SUMMARY

Provided herein are methods for generating a Streptococcus thermophilus strain exhibiting a “high galactose utilization” profile, comprising introducing a nucleic acid sequence containing the sequence set forth by SEQ ID NO:2 or a SEQ ID NO:2 derivative to a Streptococcus thermophilus strain bearing in its genome a gal-lac gene cluster and having a percentage of consumed galactose at the maximum speed of lactose consumption (VₘₐₓLacₕ) as determined by assay I which is less than 50%, and optionally a percentage of consumed galactose at the end of lactose consumption (V₀Lacₕ) determined by assay I which is less than 50%. In an embodiment, the nucleic acid sequence contains the sequence set forth by SEQ ID NO:1, 3, 4, 5, or 6 or a SEQ ID NO:1, 3, 4, 5, or 6 derivative, wherein the derivative contains the sequence set forth by SEQ ID NO:2 or the SEQ ID NO:2 derivative. In an embodiment, the introducing includes natural competence, conjugation, or transformation. In an embodiment, the method further includes after introducing the nucleic acid sequence, selecting and/or isolating one or more Streptococcus thermophilus strains which exhibit a “high galactose utilization” profile defined by a percentage of consumed galactose at the maximum speed of lactose consumption (VₘₐₓLacₕ) as determined by assay I which is at least 50%.

In an aspect is provided a Streptococcus thermophilus strain deposited under accession number DSM33851, DSM33852, DSM33853, or DSM33854 on Apr. 21, 2021, at the DSMZ or a mutant thereof.

Also provided are methods for generating a Streptococcus thermophilus strain exhibiting a “high galactose utilization” profile, including: a) providing a Streptococcus thermophilus strain, bearing in its genome a gal-lac gene cluster and having a percentage of consumed galactose at the maximum speed of lactose consumption (VₘₐₓLacₕ) as determined by assay I which is less than 50% and optionally a percentage of consumed galactose at the end of lactose consumption (V₀Lacₕ) determined by assay I which is less than 50%; b) modifying the sequence of the gal-lac gene cluster of said Streptococcus thermophilus strain to obtain a gal-lac gene cluster with a different sequence, said different sequence containing SEQ ID NO:2 or a SEQ ID NO:2 derivative; and c) selecting a Streptococcus thermophilus strain obtained in step b) which exhibits a “high galactose utilization” profile defined by a percentage of consumed galactose at the maximum speed of lactose consumption (VₘₐₓLacₕ) as determined by assay I which is at least 50%. In an embodiment, step b) is selected from the group consisting of modifying the sequence of the gal-lac gene cluster of said Streptococcus thermophilus strain to obtain a gal-lac gene cluster with a different sequence, said different sequence including SEQ ID NO:3 or a SEQ ID NO:3 derivative, and modifying the sequence of the gal-lac gene cluster of said Streptococcus thermophilus strain to obtain a gal-lac gene cluster with a different sequence, said different sequence including SEQ ID NO:4 or a SEQ ID NO:4 derivative. In an embodiment, step b) is modifying the sequence of the gal-lac gene cluster of said Streptococcus thermophilus strain to obtain a gal-lac gene cluster with a different sequence, said different sequence including SEQ ID NO:5 or a SEQ ID NO:5 derivative. In an embodiment, step b) is modifying the sequence of the gal-lac gene cluster of said Streptococcus thermophilus strain to obtain a gal-lac gene cluster with a different sequence, said different sequence including SEQ ID NO:6 or a SEQ ID NO:6 derivative, in particular the gal operon of said different sequence consists of the sequence as defined in SEQ ID NO:6 or a SEQ ID NO:6 derivative. In an embodiment, step b) is modifying the sequence of the gal-lac gene cluster of said Streptococcus thermophilus strain to obtain a gal-lac gene cluster consisting of the sequence as defined in SEQ ID NO:1 or a SEQ ID NO:1 derivative. In an embodiment, the Streptococcus thermophilus strain of step a) is galactose-negative.

In an aspect is provided a Streptococcus thermophilus strain obtainable by the methods provided herein, provided that the strain is not the Streptococcus thermophilus DSM32823 strain deposited at DSMZ on May 29, 2018.

In an aspect is provided a Streptococcus thermophilus strain characterized in that: a) the sequence of its gal-lac gene cluster contains the sequence as defined in SEQ ID NO:2 or a SEQ ID NO:2 derivative; and b) it has a “high galactose utilization” profile defined by a percentage of consumed galactose at the maximum speed of lactose consumption (VₘₐₓLacₕ) as determined by assay I which is at least 50%; provided that the strain is not the Streptococcus thermophilus DSM32823 strain deposited at DSMZ on May 29, 2018. In an embodiment, its gal-lac gene cluster is selected from the group consisting of a gal-lac gene cluster, the sequence of which includes the sequence as defined in SEQ ID NO:3 or a SEQ ID NO:3 derivative and a gal-lac gene cluster, the sequence of which includes the sequence as defined in SEQ ID NO:4 or a SEQ ID NO:4 derivative. In an embodiment, the sequence of its gal-lac gene cluster includes the sequence as defined in SEQ ID NO:5 or a SEQ ID NO:5 derivative. In an embodiment, the sequence of its gal-lac gene cluster includes the sequence as defined in SEQ ID NO:6 or a SEQ ID NO:6 derivative, in particular the gal operon of its gal-lac gene cluster consists of the sequence as defined in SEQ ID NO:6 or a SEQ ID NO:6 derivative. In an embodiment, the sequence of its gal-lac gene cluster consists of the sequence as defined in SEQ ID NO:1 or a SEQ ID NO:1 derivative.

In an embodiment, the Streptococcus thermophilus strain provided herein or obtained or obtainable by any of the methods provided herein has a “high galactose utilization” profile defined by a percentage of consumed galactose at the maximum speed of lactose consumption (VₘₐₓLacₕ) as determined by assay I which is selected from the group consisting of a percentage of at least 50%, at least 55%, at least 60%, at least at least 65%, at least 70%, at least 75%, at least 80% and at least 85%. In an embodiment, the “high galactose utilization” profile is further defined by a percentage of consumed galactose at the end of lactose consumption (V₀Lacₕ) as determined by assay I which is at least at least 70%, particularly is selected from the group consisting of a percentage of at least 70%, at least 75%, at least 80%, at least 85%, at least 90%, at least 95% and a percentage which is 100%. In an embodiment, the Streptococcus thermophilus strain provided herein or obtained or obtainable by any of the methods provided herein has a “high galactose utilization” profile defined by a percentage of consumed galactose at the maximum speed of lactose consumption (VₘₐₓLacₕ) as determined by assay I which is selected from the group consisting of a percentage of at least 50%, at least 55%, at least 60%, at least at least 65%, at least 70%, at least 75%, at least 80% and at least 85%, and a percentage of consumed galactose at the end of lactose consumption (V₀Lacₕ) as determined by assay I which is selected from the group consisting of a percentage of at least 70%, at least 75%, at least 80%, at least 85%, at least 90%, at least 95% and a percentage which is 100%. In an embodiment, the Streptococcus thermophilus strain provided herein or obtained or obtainable by any of the methods provided herein has a “high galactose utilization” profile defined by either a) a percentage of consumed galactose at the maximum speed of lactose consumption (VₘₐₓLacₕ) as determined by assay I which is selected from the group consisting of a percentage of at least 70%, at least 75%, at least 80% and at least 85%, and a percentage of consumed galactose at the end of lactose consumption (V₀Lacₕ) as determined by assay I which is selected from the group consisting of a percentage of at least 80%, at least 85%, at least 90%, at least 95% and a percentage which is 100%, or b) a percentage of consumed galactose at the maximum speed of lactose consumption (VₘₐₓLacₕ) as determined by assay I which is at least 50% and less than 70%, and a percentage of consumed galactose at the end of lactose consumption (V₀Lacₕ) as determined by assay I which is at least 70% and at most 80%. In an embodiment, a SEQ ID derivative has at least 97% identity with said SEQ ID. In an embodiment, the Streptococcus thermophilus strain provided herein or obtained or obtainable by any of the methods provided herein has a genome sequence with an identity which is at most 99.98%, at most 99.97%, at most 99.6% or at most 99.5% to the genome sequence of the DSM32823 strain. In an embodiment, the Streptococcus thermophilus strain provided herein or obtained or obtainable by any of the methods provided herein is not a variant of the DSM32823 strain.

A culture including a Streptococcus thermophilus strain provided herein or obtained or obtainable by any method provided herein is provided. In an embodiment, the culture includes at least one bacterial strain and/or ingredient(s). A kit-of-part including or consisting of a) a Streptococcus thermophilus strain provided herein or obtained or obtainable by any method provided herein and b) at least one other bacterial strain and/or ingredient(s) is also provided. In an embodiment, the at least one other bacterial strain is from the genus Lactococcus and/or Lactobacillus. In an embodiment, the at least one other bacterial strain is a Lactococcus lactis subsp. lactis, a Lactococcus lactis subsp. cremoris, and/or a Lactobacillus helveticus.

Also provided herein are food or feed products including a Streptococcus thermophilus strain provided herein or obtained or obtainable by any method provided herein or a kit-of-part or culture provided herein. In an embodiment, the food or feed product is a dairy, meat or cereal food or feed product. In an embodiment, the food or feed product is a fermented dairy food product.

Methods to manufacture a fermented product are provided, including a) inoculating a substrate with a Streptococcus thermophilus strain provided herein or obtained or obtainable by any method provided herein or a kit-of-part or culture provided herein; and b) fermenting the inoculated substrate obtained from step a) to obtain a fermented product. In an embodiment, the substrate is a milk substrate. In an embodiment, the fermented product is a fermented dairy product.

In an aspect is provided methods to manufacture pasta-filata cheese, including: a) providing or producing a curd suitable for stretching, wherein said curd is obtained by inoculating and fermenting milk with a Streptococcus thermophilus strain provided herein or obtainable by any method provided herein or a kit-of-part or culture provided herein; b) stretching the curd of step a) to obtain a stretched curd; and c) manipulating the stretched curd of step b), to finally end up with a pasta-filata cheese. In an embodiment, step a) of producing a curd suitable for stretching includes: a1) inoculating milk with a Streptococcus thermophilus strain provided herein or obtained or obtainable by any method provided herein or a kit-of-part or culture provided herein; a2) fermenting the inoculated milk of step a1) to obtain a coagulated milk; a3) cutting the coagulated milk of step a2), heating and stirring, to obtain a mix of curd and whey; and a4) draining the mix of curd and whey of step a3), to obtain a curd suitable for stretching. In an embodiment, the method further includes inoculating the milk with a milk coagulant. In an embodiment, the method further includes washing the curd, when heating and stirring at step a3).

Use of at least a Streptococcus thermophilus strain provided herein or obtained or obtainable by any method provided herein to manufacture a pasta-filata cheese is provided. Also provided is use of at least a Streptococcus thermophilus strain provided herein or obtained or obtainable by any method provided herein to produce a cheese whey which has a galactose concentration decreased as compared to a cheese whey produced using a Streptococcus thermophilus strain bearing in its genome a gal-lac gene cluster and having a percentage of consumed galactose at the maximum speed of lactose consumption (VₘₐₓLacₕ) as determined by assay I which is less than 50% and optionally a percentage of consumed galactose at the end of lactose consumption (V₀Lacₕ) determined by assay I which is less than 50%.

Each of the aspects and embodiments described herein are capable of being used together, unless excluded either explicitly or clearly from the context of the embodiment or aspect.

### DESCRIPTION OF THE DRAWINGS

FIG. 1 is a scheme representing the gal-lac gene cluster of S. thermophilus. Shaded boxes represent genes (the direction of transcription is indicated). The genes code for the following proteins: GalR, putative transcriptional regulator; GalK, galactokinase; GalT, galactose-1-P uridylyltransferase; GalE, UDP-glucose 4-epimerase; GalM, galactose mutarotase; LacS, lactose transporter; LacZ, β-galactosidase. Terminators are indicated by stem-loop structures. Arrows represent promoters (from Vaillancourt et al., 2002);

FIG. 2 is a scheme representing the Leloir pathway. Lactose is uptaken via the transporter LacS and hydrolysed into glucose and galactose by the B-galactosidase (LacZ). Glucose is phosphorylated by the phosphoglucokinase to join the glycolysis. Galactose is excreted via LacS by exchange with one molecule of lactose. For the galactose-positive strains, the galactose moiety of lactose is converted into glucose-1-phosphate (G1P) thanks to enzymes encoded by the gal operon. G1P can be redirected to glycolysis via the phosphoglucomutase (PGM) (extracted from Levander et al., 2001). EPS, exopolysaccharides; G6P, glucose 6-phosphate; GalU, UDP glucose pyrophosphorylase;

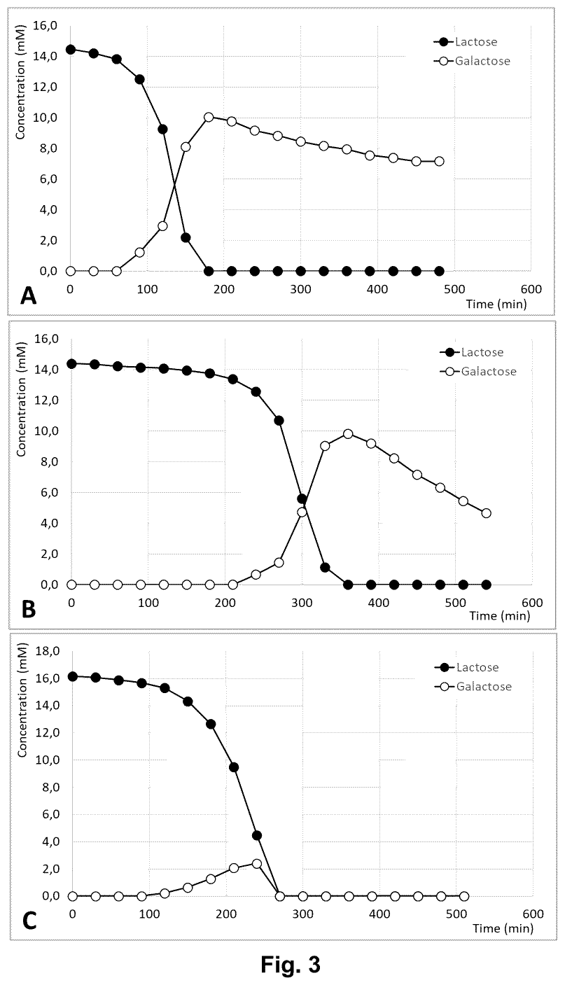

FIG. 3 represents the evolution of lactose concentration and of galactose concentration (mM) in M17 medium containing an initial concentration of 5 g/L (14.6 mM) of lactose upon fermentation with (A) strain DSM33036, (B) strain DGCC7773 and (C) strain DSM32823;

FIG. 4 is a representation of the density of single-nucleotide polymorphisms (SNPs) between 2 sequences of the gal-lac gene cluster [top] and a representation, at scale, of the open reading frames (ORFs) comprised within the gal-lac gene cluster (each arrow representing an ORF, oriented from start to stop codon, and identified by its gene name) [bottom], for (A) the comparison of SEQ ID NO:7 (DSM33036) and SEQ ID NO:8 (DGCC7773), and (B) for the comparison of SEQ ID NO:1 (DSM32823) and SEQ ID NO:7 (DSM33036);

FIG. 5 displays the evolution over time of the amount of lactose consumed (filled black circles) and of galactose consumed (empty circles) normalized to 1 litre of medium, upon fermentation by (A) strain DSM33036 and (B) strain DGCC13139;

FIG. 6 represents the speed of lactose consumption (filled black squares) and the percentage of the galactose resulting from lactose hydrolysis that is consumed over time (empty squares) upon fermentation by (A) strain DSM33036 and (B) strain DGCC13139;

FIG. 7 represents (A) the alignment of the galR-galK intergenic region of DSM32823 (SEQ ID NO:2) and DSM33036 (from SEQ ID NO:7) [the nucleotides in positions −9 and −14 of the galK promoter are underlined and the G SNP in the Shine-Dalgarno sequence is boxed], and (B) the alignment of the 200 first nucleotides of the coding sequence of the galR gene of DSM32823 (from SEQ ID NO:5) and DSM33036 (from SEQ ID NO:7) [the G nucleotide deleted in the galR coding sequence of DSM32823 as compared to the galR coding sequence of DSM33036 is boxed]. FIG. 8 shows the L values (lightness) of pizza cheese manufactured using bacterial strains as indicated after pizza baking as described in Example 8.

### DETAILED DESCRIPTION

The inventors have surprisingly shown that the galactose metabolism of Streptococcus thermophilus strains can be advantageously changed by modifying specific parts of the gal-lac gene cluster of these strains. The present application describes Streptococcus thermophilus strains presenting an advantageous galactose metabolism and a method to generate these strains, and exemplifies how this advantageous galactose metabolism can be used in various food applications.

The methods of generating Streptococcus thermophilus strains with the ability to metabolize galactose described herein are suitable for producing Streptococcus thermophilus strains useful in the food industry, for example, in the dairy industry. The methods described herein have the advantage of producing Streptococcus thermophilus strains having a “high galactose utilization” profile as described herein, which can be valuable for food production. For example, in some cases, the methods and compositions described herein provide solutions fulfilling both the requirements of food producers, e.g., cheese manufactures, in terms of time of manufacture and optionally whey valorisation, and the preferences of the food retailers and consumers (e.g., low browning).

The invention is directed to a method for generating a Streptococcus thermophilus strain having a “high galactose utilization” profile according to assay I, to a Streptococcus thermophilus strain obtained or obtainable by this method, as well as to a Streptococcus thermophilus strain per se having a “high galactose utilization” profile according to assay I.

For the avoidance of doubt, the Streptococcus thermophilus strain is to be understood as a Streptococcus salivarius subsp. thermophilus strain.

The definitions and features given within the context of the method for generating a Streptococcus thermophilus strain having a “high galactose utilization” profile according to assay I apply the same to the Streptococcus thermophilus strains obtained or obtainable by this method, and to the Streptococcus thermophilus strains per se, including, but not limited to, the sequences (SEQ ID and their derivatives) and the features defining the “high galactose utilization” profile according to assay I (such as the percentages of galactose consumption).

Unless defined otherwise, all technical and scientific terms used herein have the same meaning as commonly understood by one of ordinary skill in the art to which this disclosure belongs.

This disclosure is not limited by the exemplary methods and materials disclosed herein, and any methods and materials similar or equivalent to those described herein can be used in the practice or testing of embodiments of this disclosure.

The headings provided herein are not limitations of the various aspects or embodiments of this disclosure which can be had by reference to the specification as a whole. The section headings used herein are for organizational purposes only and are not to be construed as limiting the subject matter described. Any terms defined are more fully defined by reference to the specification as a whole.

Definitions of terms may appear throughout the specification. It is to be understood that this disclosure is not limited to particular embodiments described, as such may, of course, vary. It is also to be understood that the terminology used herein is for the purpose of describing particular embodiments only and is not intended to be limiting.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and grammatical variants thereof, are inclusive or open-ended and do not exclude additional, non-recited members, elements or method steps. The terms “comprising”, “comprises” and “comprised of”, “including”, “includes” or “containing”, “contains”, and grammatical variants thereof also include the term “consisting of”.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

The publications discussed herein are provided solely for their disclosure prior to the filing date of the present application. Nothing herein is to be construed as an admission that such publications constitute prior art to the claims appended hereto.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

Methods of Generating Streptococcus thermophilus Strains with a “High Galactose Utilization” Profile

The invention is directed to a method for generating a Streptococcus thermophilus strain having a “high galactose utilization” profile, said method comprising:
a) providing a Streptococcus thermophilus strain bearing in its genome a galactose-lactose gene cluster (gal-lac gene cluster) and having a percentage of consumed galactose at the maximum speed of lactose consumption (VₘₐₓLacₕ) as determined by assay I which is less than 50%;
b) modifying the sequence of the gal-lac gene cluster of said Streptococcus thermophilus strain to obtain a gal-lac gene cluster with a different sequence, said different sequence comprising a SEQ ID or a SEQ ID derivative as disclosed herein; and
c) selecting a Streptococcus thermophilus strain obtained in step b) which has a “high galactose utilization” profile as defined herein when tested by assay I.

In step a) of the method, a Streptococcus thermophilus strain bearing in its genome a gal-lac gene cluster and having a percentage of consumed galactose at the maximum speed of lactose consumption (VₘₐₓLacₕ) as determined by assay I which is less than 50% is provided.

The Streptococcus thermophilus strain provided in step a) of the method of the invention is characterized by at least the 2 following features:
1) the strain bears in its genome a gal-lac gene cluster

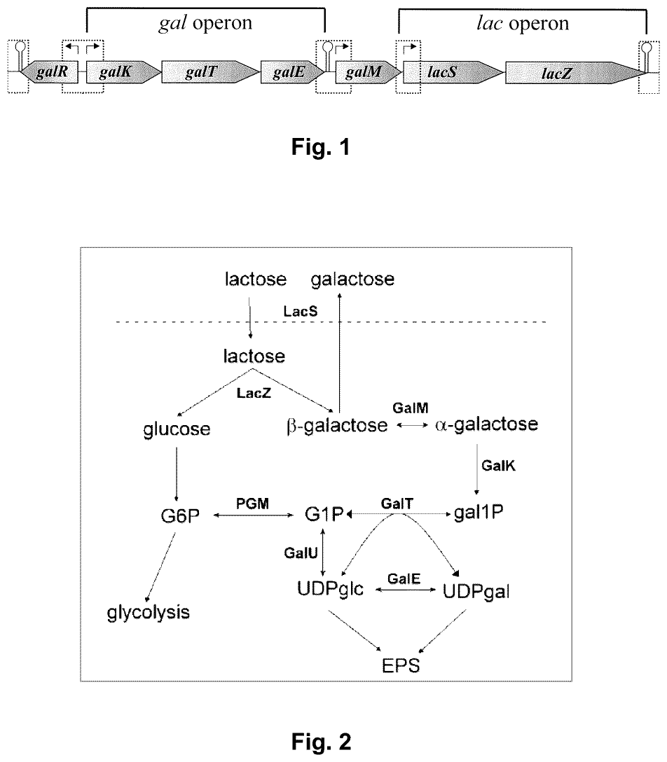

The expression “gal-lac gene cluster” as defined herein refers to a well-known cluster of genes encompassing from 5′ to 3′ the following genes: galR, galK, galT, galE, galM, lacS and lacZ (van den Bogaard et al.; System. Appl. Microbiol. 2004). The gal-lac gene cluster can be identified in the genome of S. thermophilus using the gene organization described above and based on the genes which are found upstream (an ORF encoding a putative transposase and further upstream the zmpB gene encoding a zinc-metalloprotease), and the genes which are found downstream (genes sbcC and sbcD encoding an ATP-dependent dsDNA exonuclease). In more details, the galR gene is transcribed divergently from the other genes galK, galT, galE, galM, lacS and lacZ (FIG. 1). Thus, between the galR and galK genes, an intergenic region is found, which contains both the galR and galK promoters which are divergent. The galKTEM genes are transcribed together from the galK promoter, while the galM gene can also be transcribed from a promoter region found upstream of the galM gene. The lacSZ genes are transcribed together from a promoter region found upstream of the lacS gene. The SEQ IDs disclosed herein are all in the same direction, from 5′ to 3′, which is the direction of the transcription of the galK, galT, galE, galM, lacS and lacZ genes; thus, the coding sequence of the galR gene appears as reverse complement in some of these SEQ IDs (for example its TAG stop-codon appears as CTA); and
2) the strain exhibits a percentage of consumed galactose at the maximum speed of lactose consumption (VₘₐₓLacₕ) as determined by assay I which is less than 50%. In an embodiment, the strain has a percentage of consumed galactose at the maximum speed of lactose consumption (VₘₐₓLacₕ) as determined by assay I which is less than 50% and a percentage of consumed galactose at the end of lactose consumption (V₀Lacₕ) as determined by assay I which is less than 50%. The percentages of consumed galactose at the maximum speed of lactose consumption and at the end of lactose consumption are determined by assay I as detailed herein.

### Assay I

The Streptococcus thermophilus strain to be tested is pre-cultivated twice successively in M17 broth supplemented with lactose 5 g/L for 12 hours at 37° C. The pre-culture is then used to inoculate at 1% (v/v) M17 broth supplemented with lactose 5 g/L (300-ml culture). The culture is then incubated at 37° C. in a water bath. Every 30 min, a sample of the culture is withdrawn (5 ml), filtered through a 0.2 μm Nylon filter and placed into a 2 ml HPLC vial. Filtered samples are stored at −20° C. until further analysis. Five μL of the sample are injected on an Agilent 1200 HPLC (high-performance-liquid-chromatography). The elution is done in isocratic mode with 0.025N sulfuric acid solution at 0.7 mL/min. Sugars are separated in 20 min onto an H+ ion exchange column (ROA Rezex® 150 mm×7.8 mm×8 μm) and are detected with a refractometer. Quantification is performed comparatively to an external calibration. The calculation of sugar amount is performed with Chromeleon reprocessing software (ThermoFischer Scientific). From the quantification of lactose and galactose at each time point, the percentage of galactose consumption at the maximum speed of lactose consumption (VₘₐₓLacₕ) and at null speed of lactose consumption (V₀Lacₕ) are determined as follows:
1. for each time point, the quantity of lactose consumed (Lacₕ) is determined by calculating the difference between the initial amount of lactose normalized for 1 liter of cultivation medium (Lacᵢ) minored by the amount of lactose, normalized for 1 liter of cultivation medium, upon strain growth at a given time point (Lacₜ): Lacₕ (mmol)=Lacᵢ (mmol)−Lacₜ (mmol);
2. the quantity of galactose produced at a given time point (Galₚ) is determined as being equivalent to the quantity of lactose consumed Lacₕ: Galₚ (mmol)=Lacₕ (mmol);
3. for each time point, the quantity of galactose consumed (Gal_{c}) is determined by calculating the difference between the quantity of galactose produced, normalized for 1 litre of medium, upon strain growth at a given time point (Galₚ) minored by the quantity of galactose, normalized for 1 liter of cultivation medium, upon strain growth at a given time point (Galₜ): Gal_{c} (mmol)=Galₚ (mmol)−Galₜ (mmol);
4. for each time point, the proportion of galactose consumed is determined by calculating the percentage of galactose produced from lactose that is consumed by the strain during growth: Gal_{c} (%)=[Gal_{c} (mmol)/Galₚ (mmol)]×100;
5. from the Lacₕ values calculated at each time point, the speed of lactose consumption at a given time point is determined by calculating the difference of lactose quantity between two time points divided by the duration between these two time points: VLacₕ (mmol/min)=dLacₕ (mmol)/dt (min). The time point at which the speed of lactose consumption is maximal (VₘₐₓLacₕ) and the time point at which the speed of lactose consumption reaches 0 (i.e., at the end of lactose consumption) (V₀Lacₕ) are selected;
6. the percentage of galactose [Gal_{c} (%)] calculated under 4. corresponding to the time point at which the speed of lactose consumption is maximal (VₘₐₓLacₕ) and the percentage of galactose [Gal_{c} (%)] calculated under 4. corresponding to the time point at which the speed of lactose consumption reaches 0 (V₀Lacₕ) are then respectively considered to be the percentage of galactose consumption at VₘₐₓLacₕ and the percentage of galactose consumption at V₀Lacₕ as determined by assay I for a particular Streptococcus thermophilus strain.

In a particular embodiment, the Streptococcus thermophilus strain provided in step a) of the method of the invention—which bears in its genome a gal-lac gene cluster and has a percentage of consumed galactose at the maximum speed of lactose consumption as determined by assay I which is less than 50% and optionally a percentage of consumed galactose at the end of lactose consumption as determined by assay I which is less than 50%—is galactose-negative. By the expression “galactose-negative”, it is meant a Streptococcus thermophilus strain which—when inoculated at 1% into a M17 broth containing 10% galactose and incubated for 6 hours at 42° C.—does not reduce the pH of said M17 broth to less than 6.0. In a particular embodiment, the Streptococcus thermophilus strain provided in step a) of the method of the invention—which bears in its genome a gal-lac gene cluster and has a percentage of consumed galactose at the maximum speed of lactose consumption as determined by assay I which is less than 50% and optionally a percentage of consumed galactose at the end of lactose consumption as determined by assay I which is less than 50%—is galactose-positive. By the expression “galactose-positive”, it is meant a Streptococcus thermophilus strain which—when inoculated at 1% into a M17 broth containing 10% galactose and incubated for 6 hours at 42° C.—does reduce the pH of said M17 broth to less than 6.0.

In an embodiment, the Streptococcus thermophilus strain provided in step a) of the method of the invention—which bears in its genome a gal-lac gene cluster and has a percentage of consumed galactose at the maximum speed of lactose consumption as determined by assay I which is less than 50% and optionally a percentage of consumed galactose at the end of lactose consumption as determined by assay I which is less than 50%, is unrelated to the DSM32823 strain filed at the DSMZ on May 29, 2018, i.e., that the genome sequence of the the Streptococcus thermophilus strain provided in step a) has an identity which is at most 99.97%, at most 99.96% or at most 99.95% to the genome sequence of the DSM32823 strain.

In step b) of the method, the sequence of the gal-lac gene cluster of the Streptococcus thermophilus strain provided in step a) is modified, to obtain a gal-lac gene cluster, the sequence of which is different as compared to the sequence of the gal-lac gene cluster of the Streptococcus thermophilus strain provided in step a). A gal-lac gene cluster must be obtained after the modification(s), in the sense that the general organization of the operon must be maintained. Thus, the gal-lac gene cluster obtained after the modification keeps, from 5′ to 3′, the following genes: a galR gene, a galK gene, a galT gene, a galE gene, a galM gene, a lacS gene and a lacZ gene, and keeps the original transcription organization: a galR gene which is transcribed in the reverse orientation, and galKgalT, galE, galM, lacS, and lacZ genes that are transcribed in the forward orientation. It is noteworthy that the gal-lac gene cluster obtained after step b) is located such that the ORF encoding a putative transposase and the zmpBgene encoding a zinc-metalloprotease are found upstream, and the genes sbcC and sbcD encoding an ATP-dependent dsDNA exonuclease are found downstream.

The gal-lac gene cluster thus obtained after step b), enabling to obtain a Streptococcus thermophilus strain exhibiting a “high galactose utilization” profile as defined herein when tested by assay I (step c), is further characterized as it comprises or consists of a nucleotide sequence identified herein by SEQ ID or SEQ ID derivatives. By a specific SEQ ID (e.g., SEQ ID NO: 1, 2, 3, 4, 5, or 6), it is meant the exact sequence of the SEQ ID. By “SEQ ID derivative” (e.g., SEQ ID NO: 1, 2, 3, 4, 5 or 6 derivative), it is meant a sequence which has an identity of at least 97% with said SEQ ID, wherein the identity is calculated over the whole length of the 2 sequences. In a particular embodiment, an identity of at least 97% is a percentage of identity selected from the group consisting of at least 97.5%, at least 98%, at least 98.5%, at least 99% and at least 99.5%. In an embodiment, the SEQ ID derivative differs from the SEQ ID by one or more nucleotide modification selected from the group consisting of nucleotide substitution, nucleotide deletion, nucleotide insertion and any mixture of these modification types. In an embodiment, the SEQ ID derivative differs from the SEQ ID by nucleotide substitutions (i.e. has the same size as the SEQ ID). In an embodiment, the SEQ ID derivative differs from the SEQ ID by from 1 to 30 nucleotide substitutions. In an embodiment, the SEQ ID derivative differs from the SEQ ID by from 1 to 20 nucleotide substitutions. In an embodiment, the SEQ ID derivative differs from the SEQ ID by from 1 to 15 nucleotide substitutions. In an embodiment, the SEQ ID derivative differs from the SEQ ID by from 1 to 10 nucleotide substitutions. In an embodiment, the SEQ ID derivative differs from the SEQ ID by a number of substitution(s) selected from the group consisting of 1, 2, 3, 4, 5, 6, 7, 8, 9 or 10 nucleotide substitutions. The definition of SEQ ID and SEQ ID derivative given herein in the context of the method for generating a Streptococcus thermophilus strain having a “high galactose utilization” profile applies the same to the Streptococcus thermophilus strain obtained or obtainable by the method and the Streptococcus thermophilus strain per se.

In step c) of the method, a Streptococcus thermophilus strain—whose gal-lac gene cluster is modified according to step b)—is then selected for exhibiting a “high galactose utilization” profile when tested by assay I. The expression “high galactose utilization” profile is defined herein by a percentage of consumed galactose at the maximum speed of lactose consumption (VₘₐₓLacₕ) as determined by assay I which is at least 50%. Thus, the Streptococcus thermophilus strain—whose the gal-lac gene cluster is modified according to step b)—is selected in step c) for having a percentage of consumed galactose at the maximum speed of lactose consumption (VₘₐₓLacₕ) as determined by assay I which is at least 50%. In an embodiment, the expression “high galactose utilization” profile is defined as a percentage of consumed galactose at the maximum speed of lactose consumption (VₘₐₓLacₕ) as determined by assay I which is selected from the group consisting of a percentage of at least 50%, at least 55%, at least 60%, at least 65%, at least 70%, at least 75%, at least 80% and at least 85%. In an embodiment, the expression “high galactose utilization” profile is defined as a percentage of consumed galactose at the maximum speed of lactose consumption (VₘₐₓLacₕ) as determined by assay I which is selected from the group consisting of a percentage of at least 70%, at least 75%, at least 80% and at least 85%.

In an embodiment, in addition to the percentage of consumed galactose at the maximum speed of lactose consumption as determined by assay 1, the “high galactose utilization” profile of the Streptococcus thermophilus strain of the invention is further defined by the percentage of consumed galactose at the end of lactose consumption (V₀Lacₕ) as determined by assay 1. Thus, the “high galactose utilization” profile is defined, in addition to the percentage of consumed galactose at the maximum speed of lactose consumption (VₘₐₓLacₕ) as determined by assay 1, by a percentage of consumed galactose at the end of lactose consumption (V₀Lacₕ) as determined by assay I which is at least 70%. In an embodiment, the “high galactose utilization” profile is defined, in addition to the percentage of consumed galactose at the maximum speed of lactose consumption as determined by assay 1, by a percentage of consumed galactose at the end of lactose consumption (V₀Lacₕ) as determined by assay I which is selected from the group consisting of a percentage of at least 70%, at least 75%, at least 80%, at least 85%, at least 90%, at least 95% and a percentage which is 100%. In an embodiment, the “high galactose utilization” profile is defined, in addition to the percentage of consumed galactose at the maximum speed of lactose consumption as determined by assay 1, by a percentage of consumed galactose at the end of lactose consumption (V₀Lacₕ) as determined by assay I which is selected from the group consisting of a percentage of at least 80%, at least 85%, at least 90%, at least 95% and a percentage which is 100%.

In an embodiment, the “high galactose utilization” profile of the Streptococcus thermophilus strain of the invention is defined by a percentage of consumed galactose at the maximum speed of lactose consumption as determined by assay I which is at least 50%, and a percentage of consumed galactose at the end of lactose consumption as determined by assay I which is at least 70%. In an embodiment, the “high galactose utilization” profile of the Streptococcus thermophilus strain of the invention is defined by a percentage of consumed galactose at the maximum speed of lactose consumption as determined by assay I which is selected from the group consisting of a percentage of at least 50%, at least 55%, at least 60%, at least at least 65%, at least 70%, at least 75%, at least 80% and at least 85%, and a percentage of consumed galactose at the end of lactose consumption as determined by assay I which is selected from the group consisting of a percentage of at least 70%, at least 75%, at least 80%, at least 85%, at least 90%, at least 95% and a percentage which is 100%.

In an embodiment, the Streptococcus thermophilus strain (as such or screened in step c) are characterized by a “high galactose utilization” profile defined by a percentage of consumed galactose at the maximum speed of lactose consumption as determined by assay I which is selected from the group consisting of a percentage of at least 70%, at least 75%, at least 80% and at least 85%, and a percentage of consumed galactose at the end of lactose consumption as determined by assay I which is selected from the group consisting of a percentage of at least 80%, at least 85%, at least 90%, at least 95% and a percentage which is 100%.

In another embodiment, the Streptococcus thermophilus strain (as such or screened in step c) are characterized by a “high galactose utilization” profile defined by a percentage of consumed galactose at the maximum speed of lactose consumption as determined by assay I which is at least 50% and less than 70%, and a percentage of consumed galactose at the end of lactose consumption as determined by assay I which is at least 70% and at most 80%.

For the avoidance of doubt, the percentage of consumed galactose at the maximum speed of lactose consumption and the percentage of consumed galactose at the maximum speed of lactose consumption, both as determined by assay 1, are necessarily at most 100%, since the strain cannot consume more galactose than the strain can generate from the hydrolysis of the lactose.

For the avoidance of doubt, the percentage of consumed galactose at the maximum speed of lactose consumption and the percentage of consumed galactose at the end of lactose consumption, both as determined by assay 1, are necessarily at most 100%, since the strain cannot consume more galactose than the strain can generate from the hydrolysis of the lactose.

In an embodiment, the sequence of the gal-lac gene cluster of the Streptococcus thermophilus strain provided in step a) is modified in step b) to obtain a gal-lac gene cluster with a different sequence (as compared to the gal-lac gene cluster of the Streptococcus thermophilus strain provided in step a), said different sequence comprising the sequence as defined in SEQ ID NO:2 or a SEQ ID NO:2 derivative. Thus, the sequence of the gal-lac gene cluster, once modified at step b), comprises SEQ ID NO:2 or a SEQ ID NO:2 derivative. Therefore, the invention is directed to a method for generating a Streptococcus thermophilus strain having a “high galactose utilization” profile, said method comprising:
a) providing a Streptococcus thermophilus strain bearing in its genome a gal-lac gene cluster and having a percentage of consumed galactose at the maximum speed of lactose consumption (VₘₐₓLacₕ) as determined by assay I which is less than 50%;
b) modifying the sequence of the gal-lac gene cluster of said Streptococcus thermophilus strain to obtain a gal-lac gene cluster with a different sequence, said different sequence comprising SEQ ID NO:2 or a SEQ ID NO:2 derivative; and
c) selecting a Streptococcus thermophilus strain obtained in step b) which has a “high galactose utilization” profile defined by a percentage of consumed galactose at the maximum speed of lactose consumption (VₘₐₓLacₕ) as determined by assay I which is at least 50%.

The sequence as defined in SEQ ID NO:2 consists of the intergenic region between the galR and the galK genes, i.e., the sequence located between the 1^{st} nucleotide of the start codon (ATG) of the galR coding sequence (on reverse complement) and the 1^{st} nucleotide of the start codon (ATG) of the galK coding sequence. The person skilled in the art knows based on its common general knowledge how to modify the gal-lac gene cluster of the strain provided in a) to obtain a Streptococcus thermophilus strain exhibiting a “high galactose utilization” profile according to assay I as defined herein, the gal-lac gene cluster of which comprises SEQ ID NO:2 or a SEQ ID NO:2 derivative. In an embodiment, the sequence of the gal-lac gene cluster is modified in step b) to obtain a gal-lac gene cluster with a different sequence (as compared to the gal-lac gene cluster of the Streptococcus thermophilus strain provided in step a), such that the intergenic region between the galR and the galK genes of said gal-lac gene cluster consists of the sequence as defined in SEQ ID NO:2 or a SEQ ID NO:2 derivative.

In the SEQ ID NO:2-based embodiments, it is not excluded that other part(s) of the gal-lac gene cluster is or are also modified, provided that the Streptococcus thermophilus strain to be selected in step c) exhibits a “high galactose utilization” profile according to assay I as defined herein.

In an embodiment, the sequence of the gal-lac gene cluster is modified in step b) to obtain a gal-lac gene cluster with a different sequence, such that, in addition to the modification leading to a sequence comprising SEQ ID NO:2 or a SEQ ID NO:2 derivative, the modification also encompasses the sequence located immediately upstream of SEQ ID NO:2 or the SEQ ID NO:2 derivative and/or the sequence located immediately downstream of SEQ ID NO:2 or the SEQ ID NO:2 derivative. By “immediately upstream of a SEQ ID” and “immediately downstream of a SEQ ID”, it is meant respectively the nucleotide sequence which is linked to the 5′ end of the concerned SEQ ID (here SEQ ID NO:2 or SEQ ID NO:2 derivative) and the nucleotide sequence which is linked to the 3′ end of the concerned SEQ ID (here SEQ ID NO:2 or SEQ ID NO:2 derivative). In an embodiment, when the modification concerns SEQ ID NO:2 and the sequence located immediately upstream of SEQ ID NO:2 and/or the sequence located immediately downstream of SEQ ID NO:2, the modification leads to a gal-lac gene cluster with a different sequence comprising a fragment of SEQ ID NO:1 which includes SEQ ID NO:2. By “fragment of SEQ ID NO:1 which includes SEQ ID NO:”, it is meant a fragment of consecutive nucleotides of SEQ ID NO:1 which comprises the concerned SEQ ID (here SEQ ID NO:2), and whose size ranges from the size of the concerned SEQ ID (here 141 nucleotides for SEQ ID NO:2) to 11,041 nucleotides (=SEQ ID NO:1 minus 1 nt). In an embodiment, step b) is modifying the sequence of the gal-lac gene cluster of said Streptococcus thermophilus strain of step a) to obtain a gal-lac gene cluster with a different sequence, said different sequence comprising a fragment of SEQ ID NO:1, wherein said fragment of SEQ ID NO:1 comprises SEQ ID NO:2.

In an embodiment, the sequence of the gal-lac gene cluster is modified in step b) to obtain a gal-lac gene cluster with a different sequence, such that, in addition to the modification leading to a sequence comprising SEQ ID NO:2 or a SEQ ID NO:2 derivative, the modification also encompasses other sequence(s) of the gal-lac gene cluster which is/are not located immediately upstream of SEQ ID NO:2 or the SEQ ID NO:2 derivative, and/or the located immediately downstream of SEQ ID NO:2 or the SEQ ID NO:2 derivative. This(these) other modification(s) is/are selected from the group consisting of the substitution of one or more nucleotides, the addition of one or more nucleotides, the deletion of one or more nucleotides and any mixture of these modifications, provided that the Streptococcus thermophilus strain to be selected in step c) exhibits a “high galactose utilization” profile according to assay I as defined herein. In an embodiment, this(these) other modification(s) is/are the substitution of one or more nucleotides.

In an embodiment, step b) of the method of the invention includes, but is not limited to, the replacement of the region of the intergenic region between the galR and the galK genes of the gal-lac gene cluster of the Streptococcus thermophilus strain provided in step a) by the sequence as defined in SEQ ID NO:2 or a SEQ ID NO:2 derivative. In an embodiment, step b) of the method of the invention is the replacement of the intergenic region between the galR and the galK genes of the gal-lac gene cluster of the Streptococcus thermophilus strain provided in step a) by the sequence as defined in SEQ ID NO:2 or a SEQ ID NO:2 derivative (i.e., there is no other modification of the gal-lac gene cluster than the replacement by SEQ ID NO:2 or the SEQ ID NO:2 derivative).

The invention is also directed to a Streptococcus thermophilus strain obtained or obtainable by any of the methods described above based on SEQ ID NO:2 or a SEQ ID NO:2 derivative, provided that the strain is not the Streptococcus thermophilus DSM32823 strain deposited at DSMZ on May 29, 2018. In an embodiment, the Streptococcus thermophilus strain obtained or obtainable by any of the methods described above based on SEQ ID NO:2 or a SEQ ID NO:2 derivative is not the Streptococcus thermophilus DSM32823 strain deposited at DSMZ on May 29, 2018 and is not a variant of the DSM32823 strain.

In a particular embodiment of any of the SEQ ID NO:2-based embodiments described above, the modification step b) leads to a gal-lac gene cluster with SEQ ID NO:2.

Examples of sequence which contains SEQ ID NO:2 include, but are not limited to, SEQ ID NO:3, SEQ ID NO:4, SEQ ID NO:5, and SEQ ID NO:6. In an embodiment, examples of fragment of SEQ ID NO:1 which contains SEQ ID NO:2 include, but are not limited to, SEQ ID NO:3, SEQ ID NO:4, SEQ ID NO:5, and SEQ ID NO:6. In a particular embodiment, a sequence which contains SEQ ID NO:2, optionally as a fragment of SEQ ID NO:1, is SEQ ID NO:3. In an embodiment of the invention, the sequence of the gal-lac gene cluster of the Streptococcus thermophilus strain provided in step a) is modified in step b) to obtain a gal-lac gene cluster with a different sequence (as compared to the gal-lac gene cluster of the Streptococcus thermophilus strain provided in step a), said different sequence comprising a sequence selected from the group consisting of SEQ ID NO:3, SEQ ID NO:4, SEQ ID NO:5 and SEQ ID NO:6. Therefore, the invention is directed to a method for generating a Streptococcus thermophilus strain having a “high galactose utilization” profile, said method comprising:
a) providing a Streptococcus thermophilus strain bearing in its genome a gal-lac gene cluster and having a percentage of consumed galactose at the maximum speed of lactose consumption (VₘₐₓLacₕ) as determined by assay I which is less than 50%;
b) modifying the sequence of the gal-lac gene cluster of said Streptococcus thermophilus strain to obtain a gal-lac gene cluster with a different sequence, said different sequence comprising a sequence selected from the group consisting of SEQ ID NO:3, SEQ ID NO:4, SEQ ID NO:5 and SEQ ID NO:6; and
c) selecting a Streptococcus thermophilus strain obtained in step b) which has a “high galactose utilization” profile defined by a percentage of consumed galactose at the maximum speed of lactose consumption (VₘₐₓLacₕ) as determined by assay I which is at least 50%.

The invention is not only based on any of SEQ ID NO:3, SEQ ID NO:4, SEQ ID NO:5 and SEQ ID NO:6, but also their respective derivatives as defined herein. In an embodiment of the invention, the sequence of the gal-lac gene cluster of the Streptococcus thermophilus strain provided in step a) is modified in step b) to obtain a gal-lac gene cluster with a different sequence (as compared to the gal-lac gene cluster of the Streptococcus thermophilus strain provided in step a), said different sequence comprising a sequence selected from the group consisting of SEQ ID NO:3, a SEQ ID NO:3 derivative, SEQ ID NO:4, a SEQ ID NO:4 derivative, SEQ ID NO:5, a SEQ ID NO:5 derivative, SEQ ID NO:6 and a SEQ ID NO:6 derivative. Therefore, the invention is directed to a method for generating a Streptococcus thermophilus strain having a “high galactose utilization” profile, said method comprising:
a) providing a Streptococcus thermophilus strain bearing in its genome a gal-lac gene cluster and having a percentage of consumed galactose at the maximum speed of lactose consumption (VₘₐₓLacₕ) as determined by assay I which is less than 50%;
b) modifying the sequence of the gal-lac gene cluster of said Streptococcus thermophilus strain to obtain a gal-lac gene cluster with a different sequence, said different sequence comprising a sequence selected from the group consisting of SEQ ID NO:3, a SEQ ID NO:3 derivative, SEQ ID NO:4, a SEQ ID NO:4 derivative, SEQ ID NO:5, a SEQ ID NO:5 derivative, SEQ ID NO:6 and a SEQ ID NO:6 derivative; and
c) selecting a Streptococcus thermophilus strain obtained in step b) which has a “high galactose utilization” profile defined by a percentage of consumed galactose at the maximum speed of lactose consumption (VₘₐₓLacₕ) as determined by assay I which is at least 50%.

In an embodiment, the sequence of the gal-lac gene cluster of the Streptococcus thermophilus strain provided in step a) is modified in step b) to obtain a gal-lac gene cluster with a different sequence (as compared to the gal-lac gene cluster of the Streptococcus thermophilus strain provided in step a), said different sequence comprising the sequence as defined in SEQ ID NO:3 or a SEQ ID NO:3 derivative. Thus, the sequence of the gal-lac gene cluster, once modified at step b), comprises SEQ ID NO:3 a SEQ ID NO:3 derivative. Therefore, the invention is directed to a method for generating a Streptococcus thermophilus strain having a “high galactose utilization” profile, said method comprising:
a) providing a Streptococcus thermophilus strain bearing in its genome a gal-lac gene cluster and having a percentage of consumed galactose at the maximum speed of lactose consumption (VₘₐₓLacₕ) as determined by assay I which is less than 50%;
b) modifying the sequence of the gal-lac gene cluster of said Streptococcus thermophilus strain to obtain a gal-lac gene cluster with a different sequence, said different sequence comprising SEQ ID NO:3 a SEQ ID NO:3 derivative; and
c) selecting a Streptococcus thermophilus strain obtained in step b) which has a “high galactose utilization” profile defined by a percentage of consumed galactose at the maximum speed of lactose consumption (VₘₐₓLacₕ) as determined by assay I which is at least 50%.

The sequence as defined in SEQ ID NO:3 consists of, from 5′ to 3′, part of the galR gene and the intergenic region between the galR gene and the galK gene. Thus, SEQ ID NO:3 starts by the sequence AATTGCCACTTGATACTTTT (SEQ ID NO:17) (found in the reverse complement of the galR coding sequence) and ends by the last nucleotide of the intergenic region between the galR gene and the galK gene. The person skilled in the art knows based on its common general knowledge how to modify the gal-lac gene cluster of the strain provided in a) to obtain a Streptococcus thermophilus strain exhibiting a “high galactose utilization” profile according to assay I as defined herein, the gal-lac gene cluster of which comprises SEQ ID NO:3 or a SEQ ID NO:3 derivative. In an embodiment, the sequence of the gal-lac gene cluster is modified in step b) to obtain a gal-lac gene cluster with a different sequence (as compared to the gal-lac gene cluster of the Streptococcus thermophilus strain provided in step a), such that the region of said gal-lac gene cluster starting by the sequence AATTGCCACTTGATACTTTT (SEQ ID NO:17) and ending by the last nucleotide of the intergenic region between the galR gene and the galK gene consists of the sequence as defined in SEQ ID NO:3 or a SEQ ID NO:3 derivative.

In the SEQ ID NO:3-based embodiments, it is not excluded that other part(s) of the gal-lac gene cluster is or are also modified, provided that the Streptococcus thermophilus strain to be selected in step c) exhibits a “high galactose utilization” profile according to assay I as defined herein.

In an embodiment, the sequence of the gal-lac gene cluster is modified in step b) to obtain a gal-lac gene cluster with a different sequence, such that, in addition to the modification leading to a sequence comprising SEQ ID NO:3 or the SEQ ID NO:3 derivative, the modification also encompasses the sequence located immediately upstream and/or the sequence located immediately downstream. In an embodiment, when the modification concerns SEQ ID NO:3 and the sequence located immediately upstream of SEQ ID NO:3 and/or the sequence located immediately downstream of SEQ ID NO:3, the modification leads to a gal-lac gene cluster with a different sequence comprising a fragment of SEQ ID NO:1 which includes SEQ ID NO:3. In an embodiment, step b) is modifying the sequence of the gal-lac gene cluster of said Streptococcus thermophilus strain of step a) to obtain a gal-lac gene cluster with a different sequence, said different sequence comprising a fragment of SEQ ID NO:1, wherein said fragment of SEQ ID NO:1 comprises SEQ ID NO:3. In an embodiment, the sequence of the gal-lac gene cluster is modified in step b) to obtain a gal-lac gene cluster with a different sequence, such that, in addition to the modification leading to a sequence comprising SEQ ID NO:3 or a SEQ ID NO:3 derivative, the modification also encompasses other sequence(s) of the gal-lac gene cluster which is/are not located immediately upstream of SEQ ID NO:3 or the SEQ ID NO:3 derivative and/or the located immediately downstream of SEQ ID NO:3 or the SEQ ID NO:3 derivative. This(these) other modification(s) is/are selected from the group consisting of the substitution of one or more nucleotides, the addition of one or more nucleotides, the deletion of one or more nucleotides and any mixture of these modifications, provided that the Streptococcus thermophilus strain to be selected in step c) exhibits a “high galactose utilization” profile according to assay I as defined herein. In an embodiment, this(these) other modification(s) is/are the substitution of one or more nucleotides.

In an embodiment, step b) of the method of the invention includes, but is not limited to, the replacement of the region of the gal-lac gene cluster starting by the sequence AATTGCCACTTGATACTTTT (SEQ ID NO: 17) and ending by the last nucleotide of the intergenic region between the galR gene and the galK gene of the Streptococcus thermophilus strain provided in step a) by the sequence as defined in SEQ ID NO:3 or a SEQ ID NO:3 derivative. In an embodiment, step b) of the method of the invention is the replacement of the region of the gal-lac gene cluster starting by the sequence AATTGCCACTTGATACTTTT (SEQ ID NO: 17) and ending by the last nucleotide of the intergenic region between the galR gene and the galK gene of the Streptococcus thermophilus strain provided in step a) by the sequence as defined in SEQ ID NO:3 or a SEQ ID NO:3 derivative (i.e., there is no other modification of the gal-lac gene cluster than the replacement by SEQ ID NO:3 or the SEQ ID NO:3 derivative).

The invention is also directed to a Streptococcus thermophilus strain obtained or obtainable by any of the methods described above based on SEQ ID NO:3 or a SEQ ID NO:3 derivative, provided that the strain is not the Streptococcus thermophilus DSM32823 strain deposited at DSMZ on May 29, 2018. In an embodiment, the Streptococcus thermophilus strain obtained or obtainable by any of the methods described above based on SEQ ID NO:3 or a SEQ ID NO:3 derivative is not the Streptococcus thermophilus DSM32823 strain deposited at DSMZ on May 29, 2018 and is not a variant of the DSM32823 strain.

In a particular embodiment of any of the SEQ ID NO:3-based embodiments described above, the modification step b) leads to a gal-lac gene cluster with SEQ ID NO:3.

In an embodiment, the sequence of the gal-lac gene cluster of the Streptococcus thermophilus strain provided in step a) is modified in step b) to obtain a gal-lac gene cluster with a different sequence (as compared to the gal-lac gene cluster of the Streptococcus thermophilus strain provided in step a), said different sequence comprising the sequence as defined in SEQ ID NO:4 or a SEQ ID NO:4 derivative. Thus, the sequence of the gal-lac gene cluster, once modified at step b), comprises SEQ ID NO:4 or a SEQ ID NO:4 derivative. Therefore, the invention is directed to a method for generating a Streptococcus thermophilus strain having a “high galactose utilization” profile, said method comprising:
a) providing a Streptococcus thermophilus strain bearing in its genome a gal-lac gene cluster and having a percentage of consumed galactose at the maximum speed of lactose consumption (VₘₐₓLacₕ) as determined by assay I which is less than 50%;
b) modifying the sequence of the gal-lac gene cluster of said Streptococcus thermophilus strain to obtain a gal-lac gene cluster with a different sequence, said different sequence comprising SEQ ID NO:4 or a SEQ ID NO:4 derivative; and
c) selecting a Streptococcus thermophilus strain obtained in step b) which has a “high galactose utilization” profile defined by a percentage of consumed galactose at the maximum speed of lactose consumption (VₘₐₓLacₕ) as determined by assay I which is at least 50%.

The sequence as defined in SEQ ID NO:4 consists of, from 5′ to 3′, the galR gene and the intergenic region between the galR gene and the galK gene. Thus, SEQ ID NO:4 starts by the 1^{st} nucleotide of the reverse complement of the STOP codon of the galR coding sequence and ends by the last nucleotide of the intergenic region between the galR gene and the galK gene. The person skilled in the art knows based on its common general knowledge how to modify the gal-lac gene cluster of the strain provided in a) to obtain a Streptococcus thermophilus strain exhibiting a “high galactose utilization” profile according to assay I as defined herein, the gal-lac gene cluster of which comprises SEQ ID NO:4 or a SEQ ID NO:4 derivative. In an embodiment, the sequence of the gal-lac gene cluster is modified in step b) to obtain a gal-lac gene cluster with a different sequence (as compared to the gal-lac gene cluster of the Streptococcus thermophilus strain provided in step a), such that the region of said gal-lac gene cluster starting by the 1^{st} nucleotide of the reverse complement of the STOP codon of the galR coding sequence and ending by the last nucleotide of the intergenic region between the galR gene and the galK gene consists of the sequence as defined in SEQ ID NO:4 or a SEQ ID NO:4 derivative.

In the SEQ ID NO:4-based embodiments, it is not excluded that other part(s) of the gal-lac gene cluster is or are also modified, provided that the Streptococcus thermophilus strain to be selected in step c) exhibits a “high galactose utilization” profile according to assay I as defined herein.

In an embodiment, the sequence of the gal-lac gene cluster is modified in step b) to obtain a gal-lac gene cluster with a different sequence, such that, in addition to the modification leading to a sequence comprising SEQ ID NO:4 or a SEQ ID NO:4 derivative, the modification also encompasses the sequence located immediately downstream of SEQ ID NO:4 or the SEQ ID NO:4 derivative. In an embodiment, when the modification concerns SEQ ID NO:4 and the sequence located immediately downstream of SEQ ID NO:4, the modification leads to a gal-lac gene cluster with a different sequence comprising a fragment of SEQ ID NO:1 which includes SEQ ID NO:4. In an embodiment, step b) is modifying the sequence of the gal-lac gene cluster of said Streptococcus thermophilus strain of step a) to obtain a gal-lac gene cluster with a different sequence, said different sequence comprising a fragment of SEQ ID NO:1, wherein said fragment of SEQ ID NO:1 comprises SEQ ID NO:4. In an embodiment, the sequence of the gal-lac gene cluster is modified in step b) to obtain a gal-lac gene cluster with a different sequence, such that, in addition to the modification leading to a sequence comprising SEQ ID NO:4 or a SEQ ID NO:4 derivative, the modification also encompasses other sequence(s) of the gal-lac gene cluster which is/are not located immediately downstream of SEQ ID NO:4 or the SEQ ID NO:4 derivative. This(these) other modification(s) is/are selected from the group consisting of the substitution of one or more nucleotides, the addition of one or more nucleotides, the deletion of one or more nucleotides and any mixture of these modifications, provided that the Streptococcus thermophilus strain to be selected in step c) exhibits a “high galactose utilization” profile according to assay I as defined herein. In an embodiment, this(these) other modification(s) is/are the substitution of one or more nucleotides.

In an embodiment, step b) of the method of the invention includes, but is not limited to, the replacement of the region of the gal-lac gene cluster starting by the 1^{st} nucleotide of the reverse complement of the STOP codon of the galR coding sequence and ending by the last nucleotide of the intergenic region between the galR gene and the galK gene of the Streptococcus thermophilus strain provided in step a) by the sequence as defined in SEQ ID NO:4 or a SEQ ID NO:4 derivative. In an embodiment, step b) of the method of the invention is the replacement of the region of the gal-lac gene cluster starting by the 1^{st} nucleotide of the reverse complement of the STOP codon of the galR coding sequence and ending by the last nucleotide of the intergenic region between the galR gene and the galK gene of the Streptococcus thermophilus strain provided in step a) by the sequence as defined in SEQ ID NO:4 or a SEQ ID NO:4 derivative (i.e., there is no other modification of the gal-lac gene cluster than the replacement by SEQ ID NO:4 or the SEQ ID NO:4 derivative).

The invention is also directed to a Streptococcus thermophilus strain obtained or obtainable by any of the methods described above based on SEQ ID NO:4 or the SEQ ID NO:4 derivative, provided that the strain is not the Streptococcus thermophilus DSM32823 strain deposited at DSMZ on May 29, 2018. In an embodiment, the Streptococcus thermophilus strain obtained or obtainable by any of the methods described above based on SEQ ID NO:4 or the SEQ ID NO:4 derivative is not the Streptococcus thermophilus DSM32823 strain deposited at DSMZ on May 29, 2018 and is not a variant of the DSM32823 strain.

In a particular embodiment of any of the SEQ ID NO:4-based embodiments described above, the modification step b) leads to a gal-lac gene cluster with SEQ ID NO:4.

In an embodiment, the sequence of the gal-lac gene cluster of the Streptococcus thermophilus strain provided in step a) is modified in step b) to obtain a gal-lac gene cluster with a different sequence (as compared to the gal-lac gene cluster of the Streptococcus thermophilus strain provided in step a), said different sequence comprising the sequence as defined in SEQ ID NO:5 or a SEQ ID NO:5 derivative. Thus, the sequence of the gal-lac gene cluster, once modified at step b), comprises SEQ ID NO:5 or a SEQ ID NO:5 derivative. Therefore, the invention is directed to a method for generating a Streptococcus thermophilus strain having a “high galactose utilization” profile, said method comprising:
a) providing a Streptococcus thermophilus strain bearing in its genome a gal-lac gene cluster and having a percentage of consumed galactose at the maximum speed of lactose consumption (VₘₐₓLacₕ) as determined by assay I which is less than 50%;
b) modifying the sequence of the gal-lac gene cluster of said Streptococcus thermophilus strain to obtain a gal-lac gene cluster with a different sequence, said different sequence comprising SEQ ID NO:5 or a SEQ ID NO:5 derivative; and
c) selecting a Streptococcus thermophilus strain obtained in step b) which has a “high galactose utilization” profile defined by a percentage of consumed galactose at the maximum speed of lactose consumption (VₘₐₓLacₕ) as determined by assay I which is at least 50%.

The sequence as defined in SEQ ID NO:5 consists of, from 5′ to 3′, part of the galR gene, the intergenic region between the galR and galK genes, and part of the galK gene. Thus, SEQ ID NO:5 starts by the 389^{th} nucleotide from the 1^{st} nucleotide of the reverse complement of the STOP codon of the galR coding sequence and ends by the 698^{th} nucleotide of the coding sequence of the galK gene. For the avoidance of doubt, the 389^{th} nucleotide from the 1^{st} nucleotide of the reverse complement of the STOP codon of the galR coding sequence corresponds to the 607^{th} nucleotide of the coding sequence of the galR gene. The person skilled in the art knows based on its common general knowledge how to modify the gal-lac gene cluster of the strain provided in a) to obtain a Streptococcus thermophilus strain exhibiting a “high galactose utilization” profile according to assay I as defined herein, the gal-lac gene cluster of which comprises SEQ ID NO:5 or a SEQ ID NO:5 derivative. In an embodiment, the sequence of the gal-lac gene cluster is modified in step b) to obtain a gal-lac gene cluster with a different sequence (as compared to the gal-lac gene cluster of the Streptococcus thermophilus strain provided in step a), such that the region of said gal-lac gene cluster starting by the 389^{th} nucleotide from the 1^{st} nucleotide of the reverse complement of the STOP codon of the galR coding sequence and ending by the 698^{th} nucleotide of the coding sequence of the galK gene consists of the sequence as defined in SEQ ID NO:5 or a SEQ ID NO:5 derivative.

In the SEQ ID NO:5-based embodiments, it is not excluded that other part(s) of the gal-lac gene cluster is or are also modified, provided that the Streptococcus thermophilus strain to be selected in step c) exhibits a “high galactose utilization” profile according to assay I as defined herein.

In an embodiment, the sequence of the gal-lac gene cluster is modified in step b) to obtain a gal-lac gene cluster with a different sequence, such that, in addition to the modification leading to a sequence comprising SEQ ID NO:5 or a SEQ ID NO:5 derivative, the modification also encompasses the sequence located immediately upstream of SEQ ID NO:5 or the SEQ ID NO:5 derivative and/or the sequence located immediately downstream of SEQ ID NO:5 or the SEQ ID NO:5 derivative. In an embodiment, when the modification concerns SEQ ID NO:5 and the sequence located immediately upstream of SEQ ID NO:5 and/or the sequence located immediately downstream of SEQ ID NO:5, the modification leads to a gal-lac gene cluster with a different sequence comprising a fragment of SEQ ID NO:1 which includes SEQ ID NO:5. In an embodiment, step b) is modifying the sequence of the gal-lac gene cluster of said Streptococcus thermophilus strain of step a) to obtain a gal-lac gene cluster with a different sequence, said different sequence comprising a fragment of SEQ ID NO:1, wherein said fragment of SEQ ID NO:1 comprises SEQ ID NO:5. In an embodiment, the sequence of the gal-lac gene cluster is modified in step b) to obtain a gal-lac gene cluster with a different sequence, such that, in addition to the modification leading to a sequence comprising SEQ ID NO:5 or a SEQ ID NO:5 derivative, the modification also encompasses other sequence(s) of the gal-lac gene cluster which is/are not located immediately upstream of SEQ ID NO:5 or the SEQ ID NO:5 derivative and/or the located immediately downstream of SEQ ID NO:5 or the SEQ ID NO:5 derivative. This(these) other modification(s) is/are selected from the group consisting of the substitution of one or more nucleotides, the addition of one or more nucleotides, the deletion of one or more nucleotides and any mixture of these modifications, provided that the Streptococcus thermophilus strain to be selected in step c) exhibits a “high galactose utilization” profile according to assay I as defined herein. In an embodiment, this(these) other modification(s) is/are the substitution of one or more nucleotides.

In an embodiment, step b) of the method of the invention includes, but is not limited to, the replacement of the region of the gal-lac gene cluster starting by the 389^{th} nucleotide from the 1^{st} nucleotide of the reverse complement of the STOP codon of the galR coding sequence and ending by the 698^{th} nucleotide of the coding sequence of the galK gene of the Streptococcus thermophilus strain provided in step a) by the sequence as defined in SEQ ID NO:5 or a SEQ ID NO:5 derivative. In an embodiment, step b) of the method of the invention is the replacement of the region of the gal-lac gene cluster starting by the 389^{th} nucleotide from the 1^{st} nucleotide of the reverse complement of the STOP codon of the galR coding sequence and ending by the 698^{th} nucleotide of the coding sequence of the galK gene of the Streptococcus thermophilus strain provided in step a) by the sequence as defined in SEQ ID NO:5 or a SEQ ID NO:5 derivative (i.e., there is no other modification of the gal-lac gene cluster than the replacement by SEQ ID NO:5 or the SEQ ID NO:5 derivative).

The invention is also directed to a Streptococcus thermophilus strain obtained or obtainable by any of the methods described above based on SEQ ID NO:5 or a SEQ ID NO:5 derivative, provided that the strain is not the Streptococcus thermophilus DSM32823 strain deposited at DSMZ on May 29, 2018. In an embodiment, the Streptococcus thermophilus strain obtained or obtainable by any of the methods described above based on SEQ ID NO:5 or a SEQ ID NO:5 derivative is not the Streptococcus thermophilus DSM32823 strain deposited at DSMZ on May 29, 2018 and is not a variant of the DSM32823 strain

In a particular embodiment of any of the SEQ ID NO:5-based embodiments described above, the modification step b) leads to a gal-lac gene cluster with SEQ ID NO:5.

In an embodiment of the invention, the sequence of the gal-lac gene cluster of the Streptococcus thermophilus strain provided in step a) is modified in step b) to obtain a gal-lac gene cluster with a different sequence (as compared to the gal-lac gene cluster of the Streptococcus thermophilus strain provided in step a), said different sequence comprising the sequence as defined in SEQ ID NO:6 or a SEQ ID NO:6 derivative. Thus, the sequence of the gal-lac gene cluster, once modified at step b), comprises SEQ ID NO:6 or a SEQ ID NO:6 derivative. Therefore, the invention is directed to a method for generating a Streptococcus thermophilus strain having a “high galactose utilization” profile, said method comprising:
a) providing a Streptococcus thermophilus strain bearing in its genome a gal-lac gene cluster and having a percentage of consumed galactose at the maximum speed of lactose consumption (VₘₐₓLacₕ) as determined by assay I which is less than 50%;
b) modifying the sequence of the gal-lac gene cluster of said Streptococcus thermophilus strain to obtain a gal-lac gene cluster with a different sequence, said different sequence comprising SEQ ID NO:6 or a SEQ ID NO:6 derivative; and
c) selecting a Streptococcus thermophilus strain obtained in step b) which has a “high galactose utilization” profile defined by a percentage of consumed galactose at the maximum speed of lactose consumption (VₘₐₓLacₕ) as determined by assay I which is at least 50%.

The sequence as defined in SEQ ID NO:6 consists of the gal operon (i.e., from 5′ to 3′, the galR, galK, galT, galE and galM genes). Thus, SEQ ID NO:6 starts by the 1^{st} nucleotide of the reverse complement of the STOP codon of the galR coding sequence and ends by the 3^{rd} nucleotide of the STOP codon of the galM coding sequence.

The person skilled in the art knows based on its common general knowledge how to modify the gal-lac gene cluster of the strain provided in a) to obtain a Streptococcus thermophilus strain exhibiting a “high galactose utilization” profile according to assay I as defined herein, the gal-lac gene cluster of which comprises SEQ ID NO:6 or a SEQ ID NO:6 derivative. In an embodiment, the sequence of the gal-lac gene cluster is modified in step b) to obtain a gal-lac gene cluster with a different sequence (as compared to the gal-lac gene cluster of the Streptococcus thermophilus strain provided in step a), such that the region of said gal-lac gene cluster starting by the 1^{st} nucleotide of the reverse complement of the STOP codon of the galR coding sequence and ending by the 3^{rd} nucleotide of the STOP codon of the galM coding sequence consists of the sequence as defined in SEQ ID NO:6 or a SEQ ID NO:6 derivative.

In the SEQ ID NO:6-based embodiments, it is not excluded that other part(s) of the gal-lac gene cluster is or are also modified, provided that the Streptococcus thermophilus strain to be selected in step c) exhibits a “high galactose utilization” profile according to assay I as defined herein.

In an embodiment, the sequence of the gal-lac gene cluster is modified in step b) to obtain a gal-lac gene cluster with a different sequence, such that, in addition to the modification leading to a sequence comprising SEQ ID NO:6 or a SEQ ID NO:6 derivative, the modification also encompasses the sequence located immediately downstream of SEQ ID NO:6 or the SEQ ID NO:6 derivative. In an embodiment, when the modification concerns SEQ ID NO:6 and the sequence located immediately downstream of SEQ ID NO:6, the modification leads to a gal-lac gene cluster with a different sequence comprising a fragment of SEQ ID NO:1 which includes SEQ ID NO:6. In an embodiment, step b) is modifying the sequence of the gal-lac gene cluster of said Streptococcus thermophilus strain of step a) to obtain a gal-lac gene cluster with a different sequence, said different sequence comprising a fragment of SEQ ID NO:1, wherein said fragment of SEQ ID NO:1 comprises SEQ ID NO:6. In an embodiment, the sequence of the gal-lac gene cluster is modified in step b) to obtain a gal-lac gene cluster with a different sequence, such that, in addition to the modification leading to a sequence comprising SEQ ID NO:6 or a SEQ ID NO:6 derivative, the modification also encompasses other sequence(s) of the gal-lac gene cluster which is/are not located immediately downstream of SEQ ID NO:6 or the SEQ ID NO:6 derivative. This(these) other modification(s) is/are selected from the group consisting of the substitution of one or more nucleotides, the addition of one or more nucleotides, the deletion of one or more nucleotides and any mixture of these modifications, provided that the Streptococcus thermophilus strain to be selected in step c) exhibits a “high galactose utilization” profile according to assay I as defined herein. In an embodiment, this(these) other modification(s) is/are the substitution of one or more nucleotides.

In an embodiment, step b) of the method of the invention includes, but is not limited to, the replacement of the region of the gal-lac gene cluster starting by the 1^{st} nucleotide of the reverse complement of the STOP codon of the galR coding sequence and ending by the 3^{rd} nucleotide of the STOP codon of the galM coding sequence of the Streptococcus thermophilus strain provided in step a) by the sequence as defined in SEQ ID NO:6 or a SEQ ID NO:6 derivative.

In an embodiment, step b) of the method of the invention is the replacement of the region of the gal-lac gene cluster starting by the 1^{st} nucleotide of the reverse complement of the STOP codon of the galR coding sequence and ending by the 3^{rd} nucleotide of the STOP codon of the galM coding sequence of the Streptococcus thermophilus strain provided in step a) by the sequence as defined in SEQ ID NO:6 or a SEQ ID NO:6 derivative (i.e., there is no other modification of the gal-lac gene cluster than the replacement by SEQ ID NO:6 or the SEQ ID NO:6 derivative).

The invention is also directed to a Streptococcus thermophilus strain obtained or obtainable by any of the methods described above based on SEQ ID NO:6 or a SEQ ID NO:6 derivative, provided that the strain is not the Streptococcus thermophilus DSM32823 strain deposited at DSMZ on May 29, 2018. In an embodiment, the Streptococcus thermophilus strain obtained or obtainable by any of the methods described above based on SEQ ID NO:6 or a SEQ ID NO:6 derivative is not the Streptococcus thermophilus DSM32823 strain deposited at DSMZ on May 29, 2018 and is not a variant of the DSM32823 strain.

In a particular embodiment of any of the SEQ ID NO:6-based embodiments described above, the modification step b) leads to a gal-lac gene cluster with SEQ ID NO:6.

In an embodiment, the sequence of the gal-lac gene cluster of the Streptococcus thermophilus strain provided in step a) is modified in step b) to obtain a gal-lac gene cluster with a different sequence (as compared to the gal-lac gene cluster of the Streptococcus thermophilus strain provided in step a), said different sequence consisting of SEQ ID NO:1 or a SEQ ID NO:1 derivative. Thus, the sequence of the gal-lac gene cluster, once modified at step b), consists of SEQ ID NO:1 or a SEQ ID NO:1 derivative. Therefore, the invention is directed to a method for generating a Streptococcus thermophilus strain having a “high galactose utilization” profile, said method comprising:
a) providing a Streptococcus thermophilus strain bearing in its genome a gal-lac gene cluster and having a percentage of consumed galactose at the maximum speed of lactose consumption (VₘₐₓLacₕ) as determined by assay I which is less than 50%;
b) modifying the sequence of the gal-lac gene cluster of said Streptococcus thermophilus strain to obtain a gal-lac gene cluster with a different sequence, said different sequence consisting of SEQ ID NO:1 or a SEQ ID NO:1 derivative; and
c) selecting a Streptococcus thermophilus strain obtained in step b) which has a “high galactose utilization” profile defined by a percentage of consumed galactose at the maximum speed of lactose consumption (VₘₐₓLacₕ) as determined by assay I which is at least 50%.

The sequence as defined in SEQ ID NO:1 consists of a gal-lac gene cluster. Thus, SEQ ID NO:1 starts by the 1^{st} nucleotide of the reverse complement of the STOP codon of the galR coding sequence and ends by the 3^{rd} nucleotide of the STOP codon of the lacZ coding sequence. The person skilled in the art knows based on its common general knowledge how to modify the gal-lac gene cluster of the strain provided in a) to obtain a Streptococcus thermophilus strain exhibiting a “high galactose utilization” profile according to assay I as defined herein, the gal-lac gene cluster of which consists of SEQ ID NO:1 or a SEQ ID NO:1 derivative. In an embodiment, the sequence of the gal-lac gene cluster is modified in step b) to obtain a gal-lac gene cluster with a different sequence (as compared to the gal-lac gene cluster of the Streptococcus thermophilus strain provided in step a), such that the gal-lac gene cluster, starting by the 1^{st} nucleotide of the reverse complement of the STOP codon of the galR coding sequence and ending by the 3^{rd} nucleotide of the STOP codon of the lacZ coding sequence, consists of SEQ ID NO:1 or a SEQ ID NO:1 derivative.

In an embodiment, step b) of the method of the invention is the replacement of the gal-lac gene cluster, starting by the 1^{st} nucleotide of the reverse complement of the STOP codon of the galR coding sequence and ending by the 3^{rd} nucleotide of the STOP codon of the lacZ coding sequence of the S. thermophilus strain provided in a) by the sequence as defined in SEQ ID NO:1 or a SEQ ID NO:1 derivative. In an embodiment, step b) of the method of the invention is the replacement of the gal-lac gene cluster, starting by the 1^{st} nucleotide of the reverse complement of the STOP codon of the galR coding sequence and ending by the 3^{rd} nucleotide of the STOP codon of the lacZ coding sequence of the S. thermophilus strain provided in a) by the sequence as defined in SEQ ID NO:1.

The invention is also directed to a Streptococcus thermophilus strain obtained or obtainable by any of the methods described above based on SEQ ID NO:1 or a SEQ ID NO:1 derivative, provided that the strain is not the Streptococcus thermophilus DSM32823 strain deposited at DSMZ on May 29, 2018. In an embodiment, the Streptococcus thermophilus strain obtained or obtainable by any of the methods described above based on SEQ ID NO:1 or a SEQ ID NO:1 derivative is not the Streptococcus thermophilus DSM32823 strain deposited at DSMZ on May 29, 2018 and is not a variant of the DSM32823 strain.

In a particular embodiment of any of the SEQ ID NO:1-based embodiments described above, the modification step b) leads to a gal-lac gene cluster consisting of SEQ ID NO:1.

In an aspect is provided a method for generating a Streptococcus thermophilus strain with a “high galactose utilization” profile, the method including introducing a nucleic acid sequence including the sequence set forth by SEQ ID NO:2 or a SEQ ID NO:2 derivative to a Streptococcus thermophilus strain bearing in its genome a galactose-lactose gene cluster (gal-lac gene cluster) and having a percentage of consumed galactose at the maximum speed of lactose consumption (VₘₐₓLacₕ) as determined by assay I which is less than 50%, and optionally a percentage of consumed galactose at the end of lactose consumption (V₀Lacₕ) determined by assay I of less than 50%. In an embodiment, the nucleic acid sequence includes or is the sequence of SEQ ID NO:3 or a SEQ ID NO:3 derivative. In an embodiment, the SEQ ID NO:3 derivative includes the sequence set forth by SEQ ID NO:2 or a SEQ ID NO:2 derivative. In an embodiment, the nucleic acid sequence includes or is the sequence of SEQ ID NO:4 or a SEQ ID NO:4 derivative. In an embodiment, the SEQ ID NO:4 derivative includes the sequence set forth by SEQ ID NO:2 or a SEQ ID NO:2 derivative. In an embodiment, the nucleic acid sequence includes or is the sequence of SEQ ID NO:5 or a SEQ ID NO:5 derivative. In an embodiment, the SEQ ID NO:5 derivative includes the sequence set forth by SEQ ID NO:2 or a SEQ ID NO:2 derivative. In an embodiment, the nucleic acid sequence includes or is the sequence of SEQ ID NO:6 or a SEQ ID NO:6 derivative. In an embodiment, the SEQ ID NO:6 derivative includes the sequence set forth by SEQ ID NO:2 or a SEQ ID NO:2 derivative. In an embodiment, the nucleic acid sequence includes or is the sequence of SEQ ID NO:1 or a SEQ ID NO:1 derivative. In an embodiment, the SEQ ID NO:1 derivative includes the sequence set forth by SEQ ID NO:2 or a SEQ ID NO:2 derivative. In an embodiment, the method of introducing the nucleic acid sequence is by conjugation. In an embodiment, the method of introducing the nucleic acid sequence is by transformation. In an embodiment, the method of introducing the nucleic acid sequence is by natural competence. In an embodiment, the natural competence is induced natural competence. Methods of inducing natural competence in a bacterial strain are known in the art and, in some cases, may be performed generally as described in published international application WO 2010/149721, which is incorporated by reference herein in its entirety.

In an embodiment, the Streptococcus thermophilus strain with a “high galactose utilization” profile generated by the methods in the preceding paragraph may be selected and/or isolated. In an embodiment, the Streptococcus thermophilus strain with a “high galactose utilization” profile is selected. In an embodiment, the Streptococcus thermophilus strain with a “high galactose utilization” profile is isolated, e.g., from Streptococcus thermophilus strains lacking a “high galactose utilization” profile and/or other Streptococcus thermophilus strains with a “high galactose utilization” profile. It is contemplated that the methods of the preceding paragraph may produce one or more Streptococcus thermophilus strains with a “high galactose utilization” profile that may or may not be genetically identical, e.g., in the gal-lac operon, and/or exhibit the same “high galactose utilization” profile, e.g., when assessed according to assay I. For example, it is possible that Streptococcus thermophilus strains with a “high galactose utilization” profile generated by the preceding methods display different percentages of consumed galactose at the maximum speed of lactose consumption (VₘₐₓLacₕ) as determined by assay I and/or different percentages of consumed galactose at the end of lactose consumption as determined by assay I. Thus, in some cases, Streptococcus thermophilus strains with a “high galactose utilization” profile generated according the preceding methods may be selected and/or isolated from Streptococcus thermophilus strains lacking a “high galactose utilization” profile and/or other Streptococcus thermophilus strains with a “high galactose utilization” profile that have different genotypes and/or “high galactose utilization” profiles as determined by assay I.

In an embodiment, the Streptococcus thermophilus strains with a “high galactose utilization” profile generated according to any of the methods described herein encode a truncated GalR protein. In an embodiment, the Streptococcus thermophilus strains with a “high galactose utilization” profile generated according to any of the methods described herein fail to express a GalR protein, have reduced expression of a GalR protein, or express a GalR protein with reduced or lost function, e.g., compared to a Streptococcus thermophilus strain lacking a “high galactose utilization” profile.

Streptococcus thermophilus Strains with a “High Galactose Utilization” Profile

The invention is also directed to a Streptococcus thermophilus strain which is characterized both by the nucleotide sequence of its gal-lac gene cluster and its profile according to assay I. Thus, the invention is directed to a Streptococcus thermophilus strain which is characterized in that a) the sequence of its gal-lac gene cluster comprises a SEQ ID or SEQ ID derivative as defined herein and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In an embodiment, the Streptococcus thermophilus strain is characterized in that a) the sequence of its gal-lac gene cluster comprises a SEQ ID or SEQ ID derivative as defined herein and b) it exhibits a “high galactose utilization” profile defined by a percentage of consumed galactose at the maximum speed of lactose consumption (VₘₐₓLacₕ) as determined by assay I which is at least 50% and a percentage of consumed galactose at the end of lactose consumption (V₀Lacₕ) as determined by assay I which is at least 70%, provided the strain is not the Streptococcus thermophilus DSM32823 strain deposited at DSMZ on May 29, 2018 and optionally is not a variant of the DSM32823 strain.

In an embodiment, the Streptococcus thermophilus strain of the invention is characterized in that a) the sequence of its gal-lac gene cluster comprises SEQ ID NO:2 or a SEQ ID NO:2 derivative and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In an embodiment, the Streptococcus thermophilus strain of the invention is characterized in that a) the sequence of the intergenic region between the galR and the galK genes consists of the sequence as defined in SEQ ID NO:2 or a SEQ ID NO:2 derivative, and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In a particular embodiment of any of the SEQ ID NO:2-based embodiments described above, the gal-lac gene cluster of the Streptococcus thermophilus strain of the invention comprises SEQ ID NO:2.

In an embodiment, the Streptococcus thermophilus strain of the invention is characterized in that a) the sequence of its gal-lac gene cluster comprises a part of SEQ ID NO:1 (part of SEQ ID NO:1 as defined herein), wherein said part of SEQ ID NO:1 comprises SEQ ID NO:2, and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain.

In an embodiment, the Streptococcus thermophilus strain of the invention is characterized in that a) the sequence of its gal-lac gene cluster comprises a sequence selected from the group consisting of SEQ ID NO:3, SEQ ID NO:4, SEQ ID NO:5 and SEQ ID NO:6 and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In an embodiment, the Streptococcus thermophilus strain of the invention is characterized in that a) the sequence of its gal-lac gene cluster comprises a sequence selected from the group consisting of SEQ ID NO:3, a SEQ ID NO:3 derivative, SEQ ID NO:4, a SEQ ID NO:4 derivative, SEQ ID NO:5, a SEQ ID NO:5 derivative, SEQ ID NO:6 and a SEQ ID NO:6 derivative; and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In a particular embodiment, the Streptococcus thermophilus strain of the invention is characterized in that a) the sequence of its gal-lac gene cluster comprises a sequence consisting of SEQ ID NO:4 or a SEQ ID NO:4 derivative, and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain.

In an embodiment, the Streptococcus thermophilus strain of the invention is characterized in that a) the sequence of its gal-lac gene cluster comprises a part of SEQ ID NO:1 (part of SEQ ID NO:1 as defined herein), wherein said part of SEQ ID NO:1 comprises a sequence selected from the group consisting of SEQ ID NO:3, SEQ ID NO:4, SEQ ID NO:5 and SEQ ID NO:6, and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain.

In an embodiment, the Streptococcus thermophilus strain of the invention is characterized in that a) the sequence of its gal-lac gene cluster comprises SEQ ID NO:3 or a SEQ ID NO:3 derivative; and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In an embodiment, the Streptococcus thermophilus strain of the invention is characterized in that a) the sequence of the region of its gal-lac gene cluster, starting by the sequence AATTGCCACTTGATACTTTT (SEQ ID NO: 17) and ending by the last nucleotide of the intergenic region between the galR gene and the galK gene, consists of the sequence as defined in SEQ ID NO:3 or a SEQ ID NO:3 derivative, and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In a particular embodiment of any of the SEQ ID NO:3-based embodiments described above, the gal-lac gene cluster of the Streptococcus thermophilus strain of the invention comprises SEQ ID NO:3.

In an embodiment, the Streptococcus thermophilus strain of the invention is characterized in that a) the sequence of its gal-lac gene cluster comprises the sequence as defined in SEQ ID NO:4 or a SEQ ID NO:4 derivative; and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In an embodiment, the Streptococcus thermophilus strain of the invention is characterized in that a) the sequence of the region of its gal-lac gene cluster, starting by the 1^{st} nucleotide of the reverse complement of the STOP codon of the galR coding sequence and ending by the last nucleotide of the intergenic region between the galR gene and the galK gene, consists of the sequence as defined in SEQ ID NO:4 or a SEQ ID NO:4 derivative, and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In a particular embodiment of any of the SEQ ID NO:4-based embodiments described above, the gal-lac gene cluster of the Streptococcus thermophilus strain of the invention comprises SEQ ID NO:4.

In an embodiment, the Streptococcus thermophilus strain of the invention is characterized in that a) the sequence of its gal-lac gene cluster comprises the sequence as defined in SEQ ID NO:5 or a SEQ ID NO:5 derivative, and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In an embodiment, the Streptococcus thermophilus strain of the invention is characterized in that a) the sequence of the region of its gal-lac gene cluster, starting by the 389^{th} nucleotide from the 1^{st} nucleotide of the reverse complement of the STOP codon of the galR coding sequence and ending by the 698^{th} nucleotide of the coding sequence of the galK gene, consists of the sequence as defined in SEQ ID NO:5 or a SEQ ID NO:5 derivative, and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain [for the avoidance of doubt, the 389^{th} nucleotide from the 1^{st} nucleotide of the reverse complement of the STOP codon of the galR coding sequence corresponds to the 607^{th} nucleotide of the coding sequence of the galR gene]. In a particular embodiment of any of the SEQ ID NO:5-based embodiments described above, the gal-lac gene cluster of the Streptococcus thermophilus strain of the invention comprises SEQ ID NO:5.

In an embodiment, the Streptococcus thermophilus strain of the invention is characterized in that a) the sequence of its gal-lac gene cluster comprises the sequence as defined in SEQ ID NO:6 or a SEQ ID NO:6 derivative; and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In an embodiment, the Streptococcus thermophilus strain of the invention is characterized in that a) the sequence of the gal operon of its gal-lac gene cluster, starting by the 1^{st} nucleotide of the reverse complement of the STOP codon of the galR coding sequence and ending by the 3^{rd} nucleotide of the STOP codon of the galM coding sequence, consists of the sequence as defined in SEQ ID NO:6 or a SEQ ID NO:6 derivative, and b) it exhibits a “high galactose utilization” profile defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In a particular embodiment of any of the SEQ ID NO:6-based embodiments described above, the gal-lac gene cluster of the Streptococcus thermophilus strain of the invention comprises SEQ ID NO:6.

In an embodiment, the Streptococcus thermophilus strain of the invention is characterized in that a) the sequence of its gal-lac gene cluster consists of SEQ ID NO:1 or a SEQ ID NO:1 derivative; and b) it exhibits a “high galactose utilization” profile when tested by assay I as defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In an embodiment, the Streptococcus thermophilus strain of the invention is characterized in that a) the sequence of its gal-lac gene cluster, starting by the 1^{st} nucleotide of the reverse complement of the STOP codon of the galR coding sequence and ending by the 3^{rd} nucleotide of the STOP codon of the lacZ coding sequence, consists of SEQ ID NO:1 or a SEQ ID NO:1 derivative; and b) it exhibits a “high galactose utilization” profile when tested by assay I as defined by a percentage of consumed galactose at the maximum speed of lactose consumption (VₘₐₓLacₕ) as determined by assay I which is at least 50%, provided the strain is not the Streptococcus thermophilus DSM32823 strain deposited at DSMZ on May 29, 2018 and optionally is not a variant of the DSM32823 strain. In a particular embodiment, the gal-lac gene cluster of the Streptococcus thermophilus strain of the invention consists of SEQ ID NO:1.

It is noteworthy that any of the sequences as defined in SEQ ID NO:1, SEQ ID NO:2, SEQ ID NO:3, SEQ ID NO:4, SEQ ID NO:5 and SEQ ID NO:6 can be obtained by any means known to the person skilled in the art including, but not limited to, nucleotide synthesis or amplification (such as PCR amplification) of the gal-lac gene cluster of the strain DSM32823 strain deposited at DSMZ on May 29, 2018.

As described herein, the S. thermophilus strain of the invention or obtained by the method of the invention is not the Streptococcus thermophilus DSM32823 strain deposited at DSMZ on May 29, 2018.

In an embodiment, the S. thermophilus strain of the invention or obtained by the method of the invention, in addition to be characterized by the sequence of its gal-lac gene cluster and its “high galactose utilization” profile according to assay 1, is further characterized by a genome sequence which has an identity which is at most 99.98%, at most 99.97%, at most 99.96% or at most 99.95% to the genome sequence of the DSM32823 strain. In an embodiment, the S. thermophilus strain of the invention or obtained by the method of the invention, in addition to be characterized by the sequence of its gal-lac gene cluster and its “high galactose utilization” profile according to assay 1, is further characterized by a genome sequence which has an identity which is at most 99.98%, at most 99.97%, at most 99.96% or at most 99.95% to the genome sequence of the DSM32823 strain, and which has an identity which is at least 98%, at least 98.5%, at least 99%, at least 99.1%, at least 99.2%, at least 99.3%, at least 99.4%, at least 99.5%, at least at least 99.6%, at least 99.7%, at least 99.8% or at least 99.9% to the genome sequence of the DSM32823 strain.

Within the present application, the percentage of identity of the genome of a strain to the genome of DSM23823 is defined as the percentage of the genome sequence present in the genome of the strain and found in the genome of the DSM32823 strain or the percentage of sequences present in the genome of the DSM32823 and found in the genome sequence of said strain.

In an embodiment, the S. thermophilus strain of the invention or obtained by the method of the invention is not the Streptococcus thermophilus DSM32823 strain deposited at DSMZ on May 29, 2018 and is not a variant of the DSM32823 strain.

Within the present patent application as well as within patent application PCT/EP2019/079613, a variant of the DSM32823 strain is defined as a strain obtained by mutating the DSM32823 strain (spontaneous or induced mutation step), such that the mutating strain (the variant) is genetically close to the DSM32823 strain; a strain the genome of which has an identity of at least 99.99% with the strain it derives from defines a variant. Therefore, a S. thermophilus strain of the invention, whose genome sequence has an identity which is at most 99.98% to the genome sequence of the DSM32823 strain (as defined above) is not a variant of DSM32823.

Variants of the DSM32823 strain have been more particularly defined in patent application PCT/EP2019/079613 and these variants are not part of the invention. In an embodiment, the S. thermophilus strain of the invention or obtained by the method of the invention is not the Streptococcus thermophilus DSM32823 strain deposited at DSMZ on May 29, 2018 and is not a DSM32823 variant as defined in patent application PCT/EP2019/079613. In an embodiment, the S. thermophilus strain of the invention or obtained by the method of the invention is not the Streptococcus thermophilus DSM32823 strain deposited at DSMZ on May 29, 2018 and is not a DSM32823 variant, wherein said DSM32823 variant is defined as a Streptococcus thermophilus strain:
1) presenting at least one mutation in its genome as compared to the DSM32823 strain; and
2) fulfilling at least one of these two following features regarding galactose excretion:
   2a. its ability to reach a pH of 5.2 in less than 5 hours, when inoculated at 1% (v/v) into a M17 oxoid medium supplemented with galactose 30 g/L and incubated at 43° C., in particular when tested by assay 2;
   2b. its ability not to excrete galactose, when inoculated at 1% (v/v) into a M17 medium supplemented with 0.5% (wt/vol) of lactose and incubated at 42° C., in particular when tested by assay 3 or its ability to excrete galactose but to consume the excreted galactose to completion at most 9 hours, after being inoculated at 1% (v/v) into a M17 medium supplemented with 0.5% (wt/vol) of lactose and incubated at 42° C., in particular when tested by assay 3;
and
3) fulfilling at least one of these two following features regarding its genome:
   3a. the genome sequence of the variant has an identity of at least 99.99% to the genome sequence of the DSM32823 strain;
   3b. the sequence of each of several chromosomal loci, defined according to a specific multilocus sequence typing (MLST) scheme, has an identity of least 99% with the sequence of the corresponding chromosomal locus in the DSM32823 strain. Examples of specific multilocus sequence typing (MLST) scheme are disclosed in patent application PCT/EP2019/079613.

Assay 2 (as described in PCT/EP2019/079613) is as follows:
Streptococcus thermophilus strains characterized as galactose-positive (i.e., described as able to grow on a medium comprising galactose as the only source of carbohydrates) were grown overnight at 37° C. in M17 supplemented with sucrose 30 g/L;
the culture was washed (v/v) in tryptone-salt solution (tryptone 1 g/L, NaCl 8.5 g/L) as follows: the culture was centrifuged at 4000 rpm for 5 minutes; the pellet was resuspended in 10 ml of tryptone-salt solution
the washed culture was inoculated at 1% (v/v) into 150 ml of M17 oxoid supplemented with galactose 30 g/L;
the inoculated medium was incubated at 43° C. for 24 hours, and its pH monitored using a CINAC system (Alliance Instruments, France; pH electrode Mettler 405 DPAS SC, Toledo, Spain); the pH was measured and recorded every 5 minutes. Using the CINAC v2.07 software, the time to reach a pH of 5.2 is determined.

Assay 3 (as described in PCT/EP2019/079613) is as follows:
S. thermophilus strains characterized as galactose-positive are grown 12 hours at 42° C. in M17 supplemented with 0.5% (wt/vol) of lactose [1% (v/v) inoculation]; this step is repeated a second time in the same conditions;
the culture is inoculated at 1% (v/v) into a M17 medium supplemented with 0.5% (wt/vol) of lactose, and the inoculated medium is incubated at 42° C. up to 10 hours;
during fermentation, samples are withdrawn every 30 minutes to determine the galactose concentration; samples are centrifuged at 14000×g for 5 minutes, filtered sterilized through Phenex nylon 0.45 μm-pore size×15 mm diameter filters (Phenomenex®) and stored at −20° C. until further analysis; 10 μl of each sample are injected on an Agilent®1100 HPLC. The elution is done through isocratic mode with pure H₂O at 0.6 ml/min. Sugars are separated in 40 minutes onto a Pb²⁺ ion exchange column (SP0810 Shodex™ 300 mm×8 mm×7 μm). The concentration of galactose (if any) is determined (g/L). Concentration of galactose below 0.05 g/L is considered not measurable.

In an embodiment, the Streptococcus thermophilus strains with a “high galactose utilization” profile include those deposited under accession numbers DSM33851, DSM33852, DSM33853, or DSM33854 on Apr. 21, 2021, at the DSMZ or mutants thereof. In an embodiment, the Streptococcus thermophilus strain with a “high galactose utilization” profile is the strain deposited under accession number DSM33851 at the DSMZ or mutants thereof. In embodiments, the Streptococcus thermophilus strain with a “high galactose utilization” profile is the strain deposited under accession number DSM33852, at the DSMZ or mutants thereof. In embodiments, the Streptococcus thermophilus strain with a “high galactose utilization” profile is the strain deposited under accession number DSM33853, at the DSMZ or mutants thereof. In embodiments, the Streptococcus thermophilus strain with a “high galactose utilization” profile is the strain deposited under accession number DSM33854, at the DSMZ or mutants thereof. In an embodiment, a mutant is a strain derived from a deposited strain that has been manipulated, e.g., genotypically manipulated, but maintains the same or an improved phenotype, e.g., “high galactose utilization” profile, of the parent. In an embodiment, a mutant of a Streptococcus thermophilus strain with a “high galactose utilization” profile is not the Streptococcus thermophilus DSM32823 strain deposited at DSMZ on May 29, 2018 and is not a DSM32823 variant.

### Culture and Kit-of-Part

The invention is directed to a culture comprising or consisting of a Streptococcus thermophilus strain of the invention; the expression “A culture comprising or consisting of” means that the Streptococcus thermophilus strain of the invention, when appropriate, is physically mixed together with other microorganism(s) and/or ingredients to form the culture (same pouch or same box). In an embodiment, the culture is a pure culture, i.e., comprises or consists of a single Streptococcus thermophilus strain of the invention. In an embodiment, the culture is a mixed culture, i.e. comprises or consists of a Streptococcus thermophilus strain of the invention and at least one other microorganism, in particular at least one other bacterial strain, in particular at least one other lactic acid bacterial strain, and/or ingredients. By “at least” one other (lactic acid) bacteria strain, it is meant 1 or more, and in particular 1, 2, 3, 4 or 5 strains.

The invention is also directed to a kit-of-part comprising a Streptococcus thermophilus strain of the invention; the expression “a kit-of-part comprising or consisting” means that the Streptococcus thermophilus strain of the invention and the other microorganism(s) and/or ingredients are intended to be used together but can be physically separated. In an embodiment, the kit-of-part of the invention comprising or consisting of a) a Streptococcus thermophilus strain of the invention and b) at least one other microorganism, in particular at least one other bacterial strain, in particular at least one other lactic acid bacterial strain.

In an embodiment, the culture or kit-of-part of the invention comprises or consists of the Streptococcus thermophilus strain(s) of the invention, and one or more further lactic acid bacterium of the species selected from the group consisting of a Lactococcus species, a Streptococcus species, a Lactobacillus species including Lactobacillus acidophilus, an Enterococcus species, a Pediococcus species, a Leuconostoc species, a Bifidobacterium species and an Oenococcus species or any combination thereof. Lactococcus species include Lactococcus lactis, including Lactococcus lactis subsp. lactis, Lactococcus lactis subsp. cremoris and Lactococcus lactis subsp. lactis biovar diacetylactis. Bifidobacterium species includes Bifidobacterium animalis, in particular Bifidobacterium animalis subsp lactis. Other lactic acid bacteria species include Leuconostoc sp., Streptococcus thermophilus, Lactobacillus delbrueckii subsp. bulgaricus, and Lactobacillus helveticus.

In an embodiment, the culture or kit-of-part of the invention comprises or consists of Streptococcus thermophilus strain(s) of the invention, and at least one Streptococcus thermophilus strain, different from the Streptococcus thermophilus strain(s) of the invention and/or at least one strain of the Lactobacillus species, and/or any combination thereof. In a particular embodiment, the culture or kit-of-part of the invention comprises or consists of the Streptococcus thermophilus strain(s) of the invention and one or several strain(s) of the species Lactobacillus delbrueckii subsp. bulgaricus.

In an embodiment, the culture or kit-of-part of the invention comprises or consists of a Streptococcus thermophilus strain of the invention and Lactococcus strain(s). In an embodiment, the bacterial composition comprises or consists of the Streptococcus thermophilus strain(s) of the invention, a Lactococcus lactis subsp. lactis and/or a Lactococcus lactis subsp. cremoris. In an embodiment, the culture or kit-of-part of the invention comprises or consists of a) a Streptococcus thermophilus strain of the invention, and b) Lactococcus strain(s) and/or Lactobacillus helveticus strain(s). In an embodiment, the culture or kit-of-part of the invention comprises or consists of a Streptococcus thermophilus strain of the invention and Lactobacillus helveticus strain(s). In an embodiment, the culture or kit-of-part of the invention comprises or consists of a Streptococcus thermophilus strain of the invention, Lactococcus strain(s) and Lactobacillus helveticus strain(s). In an embodiment, the culture or kit-of-part of the invention comprises or consists of a Streptococcus thermophilus strain of the invention, Lactococcus strain(s), optionally Lactococcus lactis subsp. lactis strain(s) and/or Lactococcus lactis subsp. cremoris strain(s), and Lactobacillus helveticus strain(s). In an embodiment, the culture or kit-of-part of the invention comprises or consists of a Streptococcus thermophilus strain of the invention, Lactococcus lactis subsp. lactis strain(s) and Lactobacillus helveticus strain(s). In an embodiment, the culture or kit-of-part of the invention comprises or consists of a Streptococcus thermophilus strain of the invention, Lactococcus lactis subsp. cremoris strain(s) and Lactobacillus helveticus strain(s).

In a particular of any of the embodiments defined herein, the culture and kit-of-part further comprises ingredients, such as food ingredients. In an embodiment, the food ingredient is a food acceptable ingredient, such as sugars (saccharose, trehalose), maltodextrin or minerals.

In an embodiment, and whatever their composition, the culture and kit-of-part of the invention is in frozen, dried, freeze-dried, liquid or solid format, in the form of pellets or frozen pellets, or in a powder or dried powder. In an embodiment, the culture and kit-of-part of the invention is in a frozen format or in the form of pellets or frozen pellets, in particular contained into one or more boxes or sachets. In an embodiment, the culture and kit-of-part of the invention is in a powder form, such as a dried or freeze-dried powder, in particular contained into one or more boxes or sachets. In an embodiment, when the Streptococcus thermophilus strain of the invention and other microorganisms (such as bacteria) and/or ingredients are provided as a kit of part, said Streptococcus thermophilus strain of the invention, other microorganisms (such as bacteria) and/or ingredients are under the same format, i.e, are in a frozen format, in the form of pellets or frozen pellets, a powder form, such as a dried or freeze-dried powder.

In a particular embodiment, within the culture and kit-of-part of the invention, whatever their format (frozen, dried, freeze-dried, liquid or solid format, in the form of pellets or frozen pellets, or in a powder or dried powder), the Streptococcus thermophilus strain of the invention is in a concentration comprised in the range of 10⁵ to 10¹² cfu (colony forming units) per gram (cfu/g) of the culture or of the composition of the kit-of-part into which the Streptococcus thermophilus strain is contained. In an embodiment, the concentration of the Streptococcus thermophilus strain(s) within the culture and kit-of-part of the invention is in the range of 10⁷ to 10¹² cfu per gram in particular at least 10⁷, at least 10⁸, at least 10⁹, at least 10¹⁰ or at least 10¹¹ cfu/g of the of the culture or of the composition of the kit-of-part into which the Streptococcus thermophilus strain is contained. In an embodiment, when in the form of frozen or dried concentrate, the concentration of the Streptococcus thermophilus strain of the invention within the culture and kit-of-part of the invention is in the range of 10⁸ to 10¹² cfu/g of frozen concentrate or dried concentrate, and more preferably at least 10⁸, at least 10⁹, at least 10¹⁰, at least 10¹¹ or at least 10¹² cfu/g of frozen concentrate or dried concentrate.

Product Comprising the Streptococcus thermophilus Strain of the Invention

The present invention also provides the use of the Streptococcus thermophilus strain of the invention, the culture or the kit-of-part of the invention to manufacture a food or feed product, preferably a fermented dairy product.

The invention is directed to a food or feed product comprising the Streptococcus thermophilus strain of the invention, the culture or the kit-of-part of the invention. In an embodiment, the food or feed product is a dairy, meat or cereal product. In an embodiment, a food product of the invention is a dairy product.

In an embodiment, a food product of the invention is a fermented food product. More preferably, a food as described herein is a fermented dairy product—such as a fermented milk, a yoghurt, a cream, a matured cream, a cheese, a fromage frais, a milk beverage, a processed cheese, a cream dessert, a cottage cheese, a yoghurt drink, a dairy product retentate or an infant milk. In an embodiment, the dairy product or fermented dairy product of the invention comprises milk of animal and/or plant origin. Milk is as defined elsewhere in this application.

Method to Manufacture a Product Comprising the Streptococcus thermophilus Strain of the Invention

The invention is also directed to a method for manufacturing a fermented product comprising a) inoculating a substrate with the Streptococcus thermophilus strain, the culture or the kit-of-part of the invention; and b) fermenting the inoculated substrate to obtain a fermented product.

In an embodiment, the substrate is a milk substrate, more preferably milk, and the fermented product is a fermented dairy product. By “milk substrate”, it is meant milk of animal and/or plant origin. In a particular embodiment, the milk substrate is of animal origin, in particular of any mammals, such as cow, goat, sheep, buffalo, zebra, horse, donkey, or camel, and the like. The milk may be in the native state, a reconstituted milk, a skimmed milk, or a milk supplemented with compounds necessary for the growth of the bacteria or for the subsequent processing of fermented milk. In an embodiment, the milk substrate is cow milk In an embodiment, The milk substrate is vegetable milk, that is to say extracts of plant material which have been treated or otherwise, such as leguminous plants (soya bean, chick pea, lentil and the like) or oilseeds (colza, soya bean, sesame, cotton and the like).

The invention is also directed to a fermented product, in particular a fermented dairy product, obtained or obtainable by the method for manufacturing a fermented product of the invention.

The Streptococcus thermophilus strain, the culture or the kit-of-part of the invention finds an advantageous use in various dairy applications (as particular embodiments of a method for manufacturing a fermented product described herein).

In an embodiment, the invention is directed to the use of at least the Streptococcus thermophilus strain, the culture or the kit-of-part of the invention, to manufacture a pasta-filata cheese. Thus, the invention is directed to a method to manufacture pasta-filata cheese, comprising:
a) providing or producing a curd suitable for stretching, wherein said curd is obtained by inoculating and fermenting milk with the Streptococcus thermophilus strain, the culture or the kit-of-part of the invention;
b) stretching the curd of step a) to obtain a stretched curd; and
c) manipulating the stretched curd of step b), to finally end up with a pasta-filata cheese.

Milk is as defined elsewhere in this application.

The curd provided or produced in step a) is characterized by its suitability for stretching; and the strain(s) used for producing the curd.

The term“curd” is defined herein as a curd obtained by fermentation, and therefore excludes any curd obtained by chemical acidification. As defined herein, a curd is “suitable for stretching” when the features of the curd (such as but not limited to pH, submicelle dimension) are such that the stretching step enables the curd to adopt fibers characteristics of pasta-filata cheese. These features are well-known in the art and the person skilled in the art knows which curd features are needed at this stage to manufacture a pasta-filtata cheese.

In an embodiment, the curd suitable for stretching is characterized by 1 or 2 of following features (i) a pH comprised between 4.9 and 5.4, in particular between 5 and 5.3; and/or (ii) a submicelle dimension of at least 5 nm, in particular from 5 to 15 nm, in particular from 5 to 10 nm. In a particular embodiment, the curd suitable for stretching is characterized by a pH comprised between 4.9 and 5.4, in particular between 5 and 5.3. In a particular embodiment, the curd suitable for stretching is characterized by a submicelle dimension (of at least 5 nm, in particular from 5 to 15 nm, in particular from 5 to 10 nm. In a particular embodiment, the curd suitable for stretching is characterized by a pH comprised between 4.9 and 5.4, in particular between 5 and 5.3 and a submicelle dimension of at least 5 nm, in particular from 5 to 15 nm, in particular from 5 to 10 nm.

In an embodiment, when step a) is producing a curd suitable for stretching, said step a) comprises:
a1) inoculating milk with the Streptococcus thermophilus strain, the culture or the kit-of-part of the invention, and optionally with a milk coagulant;
a2) fermenting the inoculated milk of step a1) to obtain a coagulated milk;
a3) cutting the coagulated milk of step a2), heating and stirring, to obtain a mix of curd and whey;
a4) draining the mix of curd and whey of step a3), to obtain a curd suitable for stretching.

When producing the curd upstream of step a) or as part of step a (in particular in step a2), the inoculated milk is fermented to obtain a coagulated milk. By “fermentation” (or fermenting), it is meant to keep the inoculated milk under conditions enabling lactic acid production and formation of a coagulated milk. In a particular embodiment, the inoculated milk is kept at a temperature between 30 and 42° C., in particular between 35° C. and 39° C. In an embodiment, the inoculated milk is fermented at a temperature between 30 and 42° C., in particular between 35 and 39° C.

When producing the curd upstream of step a) or as part of step a (in particular in step a3), the coagulated milk is cut, heated and stirred, to obtain a mix of curd and whey.

In an embodiment, the coagulated milk is cut, in the tank, into cubes, in particular into from 1-cm³ to 8-cm³ cubes. As an example, the cutting step lasts about 10 minutes.

In an embodiment, the cut coagulated milk is stirred and heated, in the tank, at a temperature between 38 and 42° C. In an embodiment, in combination or independently of the previous one directed to temperature, the heating and stirring is carried out until the pH of the whey reaches between 6.1 and 6.3. As an example, the heating and stirring step lasts between 10 and 30 minutes, in particular between 15 and 20 minutes.

When producing the curd upstream of step a) or as part of step a (in particular in step a4), the mix of curd and whey is drained (i.e., the whey is removed) to obtain a curd suitable for stretching as defined herein. In an embodiment, the mix of curd and whey is drained at a temperature between 38 and 42° C. The mix of curd and whey is typically drained on a draining table. As an example, the draining step lasts between 2 hours and 2.5 hours.

In step b) of the method to manufacture pasta-filata cheese of the invention, the curd (provided or produced) is stretched, to obtain a stretched curd. In an embodiment, the curd is stretched in hot water, whey or salt brine or using direct steam injection. In a particular embodiment, the curd is stretched in hot water the temperature of which is between 55 and 85° C., such that the temperature of the curd is around 50-70° C. The stretching curd is a thermo-mechanical treatment of the curd, which is typically carried out using a cooker/stretcher (such as for example but not limited to, the CMT Mozzarella Cooker Stretcher model F94). As an example, the stretching step lasts between 5 and 15 minutes.

In step c) of the method to manufacture pasta-filata cheese of the invention, the stretched curd is manipulated to finally end up with a pasta-filata cheese. Conventional steps after the stretching step include one or more of moulding (put into mould), brining (put into brine) and/or cooling.

The method to manufacture pasta-filata cheese the invention as defined herein optionally comprises additional steps: In an embodiment, the method optionally comprises washing the curd, during the heating and stirring steps. Thus, when heating and stirring the mix of curd and whey (in particular in step a3)), whey from the tank is removed and replaced by hot water (for example at 40° C.) to speed up the removal of the whey from the curd. In an embodiment, the percentage of whey removed and replaced by hot water is selected from the group consisting of 10, 20, 30 and 40%. In an embodiment, the percentage of whey removed and replaced by hot water ranges from 10 to 30%. In an embodiment, 10±3% of whey is removed and replaced by hot water. In an embodiment, 20±5% of whey is removed and replaced by hot water. In an embodiment, 30±5% of whey is removed and replaced by hot water.

Thus, in an embodiment, the invention is directed to a method to manufacture pasta-filata cheese, comprising:
a1) inoculating milk with the Streptococcus thermophilus strain, the culture or the kit-of-part of the invention, and optionally with a milk coagulant;
a2) fermenting the inoculated milk of step a1) to obtain a coagulated milk;
a3) cutting the coagulated milk of step a2), heating, stirring and washing the curd, to obtain a mix of curd and whey;
a4) draining the mix of curd and whey of step a3), to obtain a curd suitable for stretching.
b) stretching the curd to obtain a stretched curd; and
c) manipulating the stretched curd of step b), to finally end up with a pasta-filata cheese.

In an embodiment, independently or in combination with the previous one, the method to manufacture pasta-filata cheese optionally comprises, milling the curd into strips before the stretching step. Thus, in an embodiment, the invention is directed to a method to manufacture pasta-filata cheese, comprising:
a1) inoculating milk with the Streptococcus thermophilus strain, the culture or the kit-of-part of the invention, and optionally with a milk coagulant;
a2) fermenting the inoculated milk of step a1) to obtain a coagulated milk;
a3) cutting the coagulated milk of step a2), heating and stirring, and optionally washing the curd, to obtain a mix of curd and whey;
a4) draining the mix of curd and whey of step a3), to obtain a curd suitable for stretching
a5) milling the curd to obtain strips of curd
b) stretching the strips of curd to obtain a stretched curd; and
c) manipulating the stretched curd of step b), to finally end up with a pasta-filata cheese.

The invention is also directed to a stretched curd or a pasta-filata cheese or a pasta-filata cheese whey comprising the Streptococcus thermophilus strain, the culture or the kit-of-part of the invention. In an embodiment, the stretched curd or pasta-filata cheese of the invention is obtained by the method to manufacture pasta-filata cheese of the invention as defined herein. By “pasta-filata cheese whey”, it is meant a whey obtained during the manufacture of the stretched curd or pasta-filata cheese of the invention, in particular when implementing the method to manufacture pasta-filata cheese of the invention.

The invention is also directed to the use of the Streptococcus thermophilus strain, the culture or the kit-of-part of the invention to produce a cheese whey which has a galactose concentration decreased as compared to a cheese whey produced using a Streptococcus thermophilus strain bearing in its genome a gal-lac gene cluster and having a percentage of consumed galactose at the maximum speed of lactose consumption as determined by assay I which is less than 50% and optionally a percentage of consumed galactose at the end of lactose consumption determined by assay I which is less than 50%. By “cheese whey”, it is meant a whey obtained during the manufacture of a cheese. In an embodiment, the cheese whey is a pasta-filata cheese whey. In a particular embodiment, the cheese whey is a swiss-type cheese whey such as an emmental whey or maasdam whey. The cheese whey produced using the Streptococcus thermophilus strain of the invention has a galactose concentration which is decreased as compared to the galactose concentration of a cheese whey produced using a Streptococcus thermophilus strain bearing in its genome a gal-lac gene cluster and having a percentage of consumed galactose at the maximum speed of lactose consumption as determined by assay I which is less than 50% and optionally a percentage of consumed galactose at the end of lactose consumption determined by assay I which is less than 50% [with both cheese wheys produced in the same conditions, with the exception of the strains]. By “decrease” it is meant a galactose concentration (g/kg) which is decreased of at least 20% as compared to the galactose concentration of a cheese whey produced using a Streptococcus thermophilus strain bearing in its genome a gal-lac gene cluster and having a percentage of consumed galactose at the maximum speed of lactose consumption as determined by assay I which is less than 50% and optionally a percentage of consumed galactose at the end of lactose consumption determined by assay I which is less than 50%. In an embodiment, the galactose concentration (g/kg) is decreased of at least 25%. In an embodiment, the galactose concentration (g/kg) is decreased of at least 30%. In an embodiment, the galactose concentration (g/kg) is decreased of at least 40%. In an embodiment, the galactose concentration (g/kg) is decreased of a range comprised between 20% and 50%. In an embodiment, the galactose concentration (g/kg) is decreased of a range comprised between 30% and 45%. In an embodiment, the galactose concentration (g/kg) is decreased of a range comprised between 30% and 40%.

### Deposit and Expert Solution

The following deposits were made according to the Budapest treaty on the international recognition of the deposit of microorganisms for the purposes of patent procedure.
Streptococcus thermophilus strain (DGCC7698) deposited under accession number DSM32823 on May 29, 2018, at the DSMZ [Deutsche Sammlung von Mikroorganismen und Zellkulturen GmbH, Inhoffenstrasse 7B, D-38124 Braunschweig—Germany] by DuPont Nutrition Biosciences ApS
Streptococcus thermophilus strain (DGCC715) deposited under accession number DSM33036 on Feb. 12, 2019, at the DSMZ by DuPont Nutrition Biosciences ApS
Streptococcus thermophilus strain (DGCC13392) deposited under accession number DSM 33851 on Apr. 21, 2021, at the DSMZ by DuPont Nutrition Biosciences ApS
Streptococcus thermophilus strain (DGCC13393) deposited under accession number DSM 33852 on Apr. 21, 2021, at the DSMZ by DuPont Nutrition Biosciences ApS
Streptococcus thermophilus strain (DGCC13400) deposited under accession number DSM 33853 on Apr. 21, 2021, at the DSMZ by DuPont Nutrition Biosciences ApS
Streptococcus thermophilus strain (DGCC13401) deposited under accession number DSM 33854 on Apr. 21, 2021, at the DSMZ by DuPont Nutrition Biosciences ApS.

It is requested that the biological material shall be made available only by the issue of a sample to an expert nominated by the requester. In respect to those designations in which a European Patent is sought, a sample of the deposited microorganism will be made available until the publication of the mention of the grant of the European patent or until the date on which application has been refused or withdrawn or is deemed to be withdrawn, only by the issue of such a sample to an expert nominated by the person requesting the sample, and approved either i) by the Applicant and/or ii) by the European Patent Office, whichever applies (Rule 32 EPC).

SEQUENCES

SEQ ID NO: 1
ctattttgtactgtttcttaatgttaattttgttcctagtttagtaagtgttggtatttttcgaggtgctaagaa
ttgtttattgagaatatccatagctgttcgtcccatttcttctgtatagacagtgacactagaaagtggaggata
cacttgtttagctagagttgtatcgttaaaagagataatttgaatgtcgtcagggaccttgataccattttcctg
aagtgctctgagtgcaccaatagctagactatcactagccgcaaagtaagcatcaggtaaagtagctccactctt
aatcttagaatcaagaagttcatagccagattggacagtgaagtcgccagtcagaataaaaagagggtcatagat
tccctttttcatacagtagttccgataagaacgtaaacgaggatcagagatgatttcagttgcatctgttgtttt
ttcttgcccaatgagtaggcctatattattacaaccttgttctttaagataacagagtgccgattgtacggagtt
ttcaaaatcagtagtaacacatggatgtccttgattaagagtatcactatcaacaaagaccagagtcttttttag
tctttctagtttcgcaatttgttcacgactaaattttccgatacagagtacaccaacgacctcctctcctagact
tgaaggaatgtcgttgaaaaagtgaagcatctcgtagtctagttcataggctcttttttcaataccaagtctaat
attataatagtagatgtcatctaactcgtgttcttcactaacccattgaataattgccacttgatactttttttt
tttgaattattaattgttttgtatttagtatatccgatttcatcagcgatagttaatatccgatgtctagtatcc
tctgttacggaaagagtttcatctttattaagaacacgtgaaacagttgaaatagatacacctgctaagtttgcg
atatctgttaatgtagccatagtatcctccgcatatttcagtataacataacttttcttttttacctatatttta
ctaaaaaaatagtaaaagtattgatttttcatgtgaaagggattacaatttcagtgtaaacaaaaagaataaggg
agatacagcctatgaatacgttacagttaagagaaaagtttaaagaagtttttggtgtagaagcagatcatactt
tcttttcaccaggtcgtattaatttgattggtgagcatacggactacaatggaggtaacgtccttccggtagcta
ttaccctaggtacttacggagcggcccgcaaacgtgatgacaaagttttgcgtttcttctcagctaactttgaag
agaagggaatcatcgaagtgccacttgaaaatcttcgttttgaaaaagaacacaactggacaaactatccaaaag
gtgttcttcatttcttgcaagaagctgggcatacgattgattcaggtatggatatttacatctatggtaacattc
caaatggatcaggcttgtcatcatcatcatctttggaattgttgattggtgttattgctgaaaaactttatgacc
ttaaattggaacgccttgacttggttaaaatcggtaagcaaactgaaaatgactttatcggcgttaactctggta
tcatggaccaattcgctattggtatgggagctgatcaatgtgcgatttacttggatacaaatactctaaagtatg
acttgataccccttgaccttaaagacaatgttgttgttatcatgaacactaacaaacgtcgtgaattggctgatt
ctaaatacaatgaacgtcgtgctgaatgtgaaacagcagtatctgaactccaagaaaaacttgatatccaaacac
ttggtgaattggacctctggacattcgatgcatacagctacttgattaaagataaaaatcgtatcaaacgtgcac
gccatgcagttcttgaaaatcaacgtacacttcaagcccgtaaagctcttgaagcaggagatttggaaggctttg
gtcgtcttatgaatgcttctcacgtatcattggaacatgattacgaagttacaggtcttgaacttgatactttgg
cacacacagcatgggaacaagaaggtgttcttggagctcgtatgacaggagcgggatttggtggatgtgccatcg
cacttgtaaacaaagataaagttgaagatttcaaaaaagcagttggtcaacgttatgaagaagtcgttggttatg
caccaagcttctatatcgctgaagtagctggcggttcacgagtacttgattaatgagaaagaagtagagattatg
gctgaaaatttagtaaacacttttgtaactcaggttattgaaaatagtgattatgaggaattggaccgtatctat
ttgacaaacaaagtttttgctttggttggagaaggagttgctgatgttgaaacagatagctctgaattgattgac
cttaaagaccagttgcttcaagcaggtgttaaagctggttctgtaggtgaacttaatgaagaacaagatatcatc
ggtgctcaactcatggacctgattacaccaagtcctagtgttgtaaatcgtaacttttgggatacctataaatct
aatccagaacaagctatcgctgatttttatgctctcagtaagcgcaatgattatgttaaggtaaaggcaattgct
caaaatatcgcctataagtctccaacaaagtacggtgacttggaaattacgattaacctttcaaaacctgaaaaa
gatccaaaagccatcgctgcagctaaaaatgcggtagcttctgattatccaaaatgccaactttgtatggaaaat
gaaggttatctaggtcgcatcaatcacccagcacgtagtaatcaccgtgttattcgtttccaaatggaagacaag
gagtggggcttccaatattcaccatatgcttactttaatgaacattctattttcttttatggtaagcacgaacca
atgcacatcagtccattgacgtttggtcgtctcctatcaattgttgaagcatttcctggttactttgcaggttca
aatgccgatcttccaattgtaggtggttcaattcttacacacgaacactatcaaggtggtcgccacaccttccca
atggaagtagcaggcattaaagaaaaagttagctttgatggatactctgatgttgaaactggtattgttaattgg
cctatgtcagttcttcgtttaagaagtgaagataaggaaagacttattgctcttgcaaccaaaattttaaattgc
tggcgtggttattcagacgaaaaagctggtgtcttggctgagtctgatggacaacctcaccacaccattacccca
attgctcgtagaaaagacggcaaatttgaattggatttggttcttcgtgacaatcaaacttctgaagaacatcca
gacggtatctaccacccacataaagatgttcaacatattaagaaagaaaacatcggtttgattgaagttatggga
ttggccattcttccacctcgtttgaaaacagaacttaaagatgttgaagattatctattgggtcaaggtaaccaa
gttgctccaattcaccaagaatgggcagatgaactcaaagctcaaaatccgaatgttacggctgaggaagtgaca
gaagttgttcgacaatctgttgcagatatctttgctcgtgtactagaagatgcaggtgtttataagactaatagt
gaaggcttggatcagtttaaagcatttgtagattttgtaaatttagctgattaattgttttttctgaagaaagga
gataagaaatggcaattttagtattaggtggagctggttatatcggttctcacatggttgatcgcttagttgaaa
aaggtcaagaaaaagtagtagtagtagatagtttggtaaccggtcaccgtgctgccgtacatccagatgctattt
tctatcaaggcgatctctctgatcaagattttatgagaaaggtcttcaaagaaaatcctgatgttgatgccgtta
ttcactttgcggcctattcattggttggtgaatcaatggaaaaaccactcaaatattttgataacaatacagctg
gaatggttaaattgcttgaagttatgaacgaatgcggtgttaaatacattgtcttctcatcaacagcagcaactt
acggaattccagaagaaattccaattcttgaaacaacaccacaaaatcctatcaacccatatggtgaaagtaagt
tgatgatggaaaccattatgaagtggtctgataaagcttatggtattaaatatgtgccacttcgttacttcaacg
ttgctggtgcaaaacctgatggatctatcggtgaagaccatggtccagaaactcaccttcttccaattatccttc
aagtagctcaaggtgttcgtgaaaaaatcatgatctttggggatgactacaatactccagacgggactaacgttc
gtgactatgtgcatccattcgacttggcggacgctcacctccttgctgttgaatatcttcgtaaaggtaatgaat
ctacagcctttaacttaggttcatcaacaggtttttcaaaccttcaaattcttgaagcagctcgtaaggtaactg
gtaaagagattccagctgaaaaagttgatcgtcgtcctggtgatcctgatatattgattgcttcatctgaaaaag
cacgtacagttcttggatggaaaccacaatttgataacatcgaaaaaatcatcgcaagtgcttgggcatggcatt
ctagccatccaaaagggtacgatgatcgaggataattaaaataataaacaaaaataaagagtatatataattgtt
gtacttctttgtttttttgaggaaataatatgaaaataagctgcgaaattattggaaaagttgattcaggcgatg
tcagtaaaatttcaatggaaaataataatggtgttgtcatttccacactcacaacaggtgctacacttcaggagt
ttttggttccaacggaaactggtgctcttaaaaatatagtacttggtttcagtgatttcgaggattactataaga
ataatttatgtgcctgccagtccattggtagggttgctggaagaattgggaaagcttcgtatactcataatatgg
ttctttatagccttcctaagaatgagggagaaaactgcttacatggcggtccaaaaggaatgcaggttcaaaatt
ggaactatgtcacaaacctgaatgatgaatatgttgagacaaaatttattagaagactttattcaagtgtagatg
gctttcctggtgatgttacagtctctattagttatagacttgacaataataaccgcttaacaatactctttgaag
ccttcgatgttacagagtcaacagtctttaatccgacaaaccatgtttactttaatcttagcgacaaacaggatt
tatcaagtcatgagttacaaatctattcagactatcgtttagagttggattctgagttaatccctacaggacaaa
aaattaatgtagatggaacaaattatgactttagaaagacaactgacttgctacctcgaattgaagcaaataatg
gttttgatgatgcctttgtagttgaagggggaacttgtgatcatgtgaaagaagtagctattttgcatgacaaag
aaagcggagatggtattgaaatctactcaaacagaaatggtttagtcatttttacgatggatgatatcgaagata
atatttactttgcaagagataaaggcaaaatggcaaaagggcgtgaagctattgctctggaagctcaaacccttc
cagatgccgttaatcacaaaggttttggagatattatcttaaataaaggccatagtgtaaactatgaaattggct
tccaatactttaattcatcaagataaccatgtattagtaaaagtttagtaaaaaacactgaaattattgactgta
taaaccaatttttatataatgtaaacgcattcaaataataggaggtttccgaaatggaaaaatctaaaggtcaga
tgaagtctcgtttatcctacgcagctggtgctttcggtaacgacgtcttctatgcaaccatgtcaacatacttta
tcatgtttgtgacaactcacttgtttaacacaggtgatccaaagcaagatagtcactacgtactattaattacta
acattatcgctgctttgcgtatcttggaagtatttatcgatccattgatcggtaatatgattgataacactaata
ctaagtatggtaaattcaaaccatgggtagttggtggtggtatcatcagttctatcaccttgttgcttctcttca
ccgatttaggtggtttgaataaaacaaatcctttcttgtaccttgtactttttggaattatctaccttgtaatgg
atgtcttctactcgattaaagatatcggtttctggtcaatgattcctgccttgtctcttgatagtcacgaacgtg
aaaaaatggcaacttttgcccgtattggttctacgattggtgccaatattgtaggtgttgccatcatgccaatcg
ttttgttcttctctatgacgaacaatagtggctctggagataaatctggatggttctggtttgcatttatcgttg
ctctcattggtgtgattacatcaattgctgttggtattggtacacgtgaagttgagtcaaaacttcgtgataaca
atgaaaaaactagccttaaacaagtctttaaagttcttggtaaaaacgaccaattgatgtggctgtctctcggat
attggttctatggtcttggtattaatacacttaatgctcttcaactttattacttcacatttatccttggtgatt
cagggaaatactcaatcctttacggattgaatacagttgttggtttggtttcagtttcactgttccctagtcttg
ctggtaaattcaaccgtaaacgtttgttctatggatgtattgcagtaatgctcggtggtatcggaatctttagta
ttgcaggtacttcacttccaatgatcttgactgcagctgaactcttcttcattccacaacctcttgtgttccttg
ttatccttatgattatctctgactcagtagaatatggtcaatggaaattgggacaccgtgatgaatcacttactt
tgtcagttcgtccacttgttgataaacttggtggtgcgatgtcaaactggcttgtttctacaattgccgtagctg
ccggtatgacaacaggtgcctcagcatcaacaattacaacacatcaacagtctatttttaagcttagcatgtttg
gtttcccagcagcagcaatgcttatcggtgccttcattattgctcgtaaaatcactttgactgaagcacgtcacg
ctgaaattgttgaagaattggaaaatcgctttagcgtagcaacttctgaaaatgaagttaaagctaacgtcgtat
ctcttgtagcccctacaactggtaaactagttgatctttcaagcgttagtgataaacgctttgcttcaggtagcc
taggtaaaggatttgcaatcaaaccttcagatggtaaggtttttgccccaattagtggtacagttcgtcagattc
tacctactcgtcatgcgattggtattgaaagtgatgatggtgtcgttgttctcgtacacgttggtattggaacag
ttaaacttaaaggtgaagggttcattagttacgtaaaacaaggtgatcgtgttgaagttggacaaaaacttcttg
agttctggtcaccaattattgagaaaaatggtcttgatgacacagtacttgtcactgtaactaattcagaaaaat
tcagtgctttccatcttgaacaaaaagttggagaaaaggtagaagctttgtctgaagttattaccttcaaaaaag
gagaataatttatgaacacgactgaaaaaattcaaacttacttaaacgatccaaagattgttagcgttaatactg
ttgacgctcactcagatcataagtattttgaatctcttgcagaattttctgagggggagatgaagttaagacaat
ctcttaatggaaaatggaaaattcactatgctcagaatacaaatcaggttttaaaagacttttataaaacagaat
ttgatgaaactgatttgaatttcatcaatgtaccaggtcatttagagcttcaaggttttggttctccacaatatg
tgaatacccagtatccttgggatggtaaagaattccttcgtccacctcaagttcctcaagaatcaaatgctgttg
catcatacgttaaacattttaccttgaatgatgcattaaaagataaaaaagtatttatctcattccaaggggttg
ctacttccatctttgtatgggtcaatggtaacttcgtaggatacagtgaagattcatttacacctagtgaatttg
aaattagtgattaccttgttgaaggtgataacaagttggcggtagctgtttatcgttactctacagcaagctggt
tggaagaccaagacttctggagactttacggtatttttagagatgtttacttgtatgctattccaaaagttcacg
ttcaagatctctttgttaagggagattatgattaccaaacaaaaacaggtcaattggatattgatttgaagactg
ttggtgattatgaagacaagaagattaaatatgttctttcagattatgaaggcatcgttacagaaggtgatgcat
ctgttaatggtgacggtgaactatctgtaagtcttgaaaatcttaaaatcaaaccttggagtgctgaaagtccta
aactttacgatttgatccttcatgttttggatgatgaccaagttgttgaagtcgttccagttaaagttggattta
gacgctttgaaattaaagataaacttatgcttttgaatggtaagagaattatctttaaaggggttaacagacacg
aatttaacgctagaacaggacgttgtatcactgaagaagatatgctttgggatatcaaagtgatgaaacaacata
acatcaatgctgttcgtacttcacactatcctaaccaaacacgttggtatgaattgtgtgatgaatatggacttt
atgttatcgatgaagccaaccttgaaacacacggtacatggcaaaaacttggtctatgcgaaccttcatggaata
tcccagctagtgaaccagaatggttgcctgcttgtttggatcgtgccaataacatgttccaacgcgataagaacc
acgctagtgttatcatttggtcttgtggtaatgaatcatatgctggtaaagatattgctgacatggctgattact
tccgtagtgttgacaatactcgtccagttcactatgaaggtgttgcatggtgtcgtgagtttgattacattacag
acatcgaaagtcgtatgtatgcgaaaccagctgatatcgaagaatacctcacaactggtaaactagttgatcttt
caagcgttagtgataaacactttgcttcaggtaacctaactaacaaacctcaaaaaccttatatttcatgtgaat
acatgcacacaatgggtaactctggtggtggattgcaactctacactgacttagagaaatatccagaataccaag
gtggatttatttgggacttcattgaccaagctatttacaaaacacttccaaatggtagcgaattcctatcatatg
gtggtgactggcatgatagaccttctgactacgaattttgtggaaatggtatcgtctttgcagatcgtaccctaa
ctccaaaacttcaaacagttaaacatctttactctaatattaagattgctgttgatgaaaaatcagtaactatca
agaatgataatctcttcgaagatctttctgcttatactttcctagctagagtttacgaagatggtagaaaagtta
gtgaaagtgaatatcactttgatgttaaaccaggcgaagaagcaacattcccagttaactttgtagttgaggctt
caaattctgaacaaatttacgaagttgcttgtgttctgagggaagcaactaaatgggctcctaaaggtcatgaaa
ttgttcgtggtcaatatgttgttgaaaagattagcactgaaacaccagttaaagcacctttgaatgttgttgaag
gggacttcaacatcggtattcaaggacaaaacttctcaatcttgctttcacgtgcacaaaatactttagtatctg
ctaagtataatggtgttgaattcattgagaaaggtcctaaacttagcttcactcgtgcttacactgacaacgatc
gtggtgctggatatccattcgaaatggcaggctggaaggttgctggaaactatagtaaagttacagatactcaaa
ttcaaatcgaagacgactctgttaaagtgacttatgttcatgaattgccaggcttgtctgatgtcgaagttaagg
taacttatcaagttgactacaagggtcgaatctttgttactgcaaactatgatggtaaagcaggtttgccaaact
tccctgaatttggtctagaatttgctatcggttcacaatttacaaaccttagctattatggatacggtgcagaag
aaagctaccgtgataaacttcctggtgcctatcttggtcgatatgaaacatctgttgaaaagacatttgctccat
atctaatgccacaagaatctggtaatcactatggtactcgtgaattcacagtatctgatgataaccataatggtc
ttaaattcaccgcacttaataaagcattcgaattcagtgctttgcgtaacagtactgaacaaattgaaaatgctc
gtcaccaatatgagttgcaagaatctgatgctacatggattaaagttcttgctgctcaaatgggtgtaggtggtg
acgactcatggggtgctccagttcatgacgaattcttgcttagctcagcagatagctatcaattaagcttcatga
ttgaaccactaaattag
SEQ ID NO: 2
agtatcctccgcatatttcagtataacataacttttcttttttacctatattttactaaaaaaatagtaaaagta
ttgatttttcatgtgaaagggattacaatttcagtgtaaacaaaaagaataagggagatacagcct
SEQ ID NO: 3
aattgccacttgatactttttttttttgaattattaattgttttgtatttagtatatccgatttcatcagcgata
gttaatatccgatgtctagtatcctctgttacggaaagagtttcatctttattaagaacacgtgaaacagttgaa
atagatacacctgctaagtttgcgatatctgttaatgtagccatagtatcctccgcatatttcagtataacataa
cttttcttttttacctatattttactaaaaaaatagtaaaagtattgatttttcatgtgaaagggattacaattt
cagtgtaaacaaaaagaataagggagatacagcct
SEQ ID NO: 4
ctattttgtactgtttcttaatgttaattttgttcctagtttagtaagtgttggtatttttcgaggtgctaagaa
ttgtttattgagaatatccatagctgttcgtcccatttcttctgtatagacagtgacactagaaagtggaggata
cacttgtttagctagagttgtatcgttaaaagagataatttgaatgtcgtcagggaccttgataccattttcctg
aagtgctctgagtgcaccaatagctagactatcactagccgcaaagtaagcatcaggtaaagtagctccactctt
aatcttagaatcaagaagttcatagccagattggacagtgaagtcgccagtcagaataaaaagagggtcatagat
tccctttttcatacagtagttccgataagaacgtaaacgaggatcagagatgatttcagttgcatctgttgtttt
ttcttgcccaatgagtaggcctatattattacaaccttgttctttaagataacagagtgccgattgtacggagtt
ttcaaaatcagtagtaacacatggatgtccttgattaagagtatcactatcaacaaagaccagagtcttttttag
tctttctagtttcgcaatttgttcacgactaaattttccgatacagagtacaccaacgacctcctctcctagact
tgaaggaatgtcgttgaaaaagtgaagcatctcgtagtctagttcataggctcttttttcaataccaagtctaat
attataatagtagatgtcatctaactcgtgttcttcactaacccattgaataattgccacttgatactttttttt
tttgaattattaattgttttgtatttagtatatccgatttcatcagcgatagttaatatccgatgtctagtatcc
tctgttacggaaagagtttcatctttattaagaacacgtgaaacagttgaaatagatacacctgctaagtttgcg
atatctgttaatgtagccatagtatcctccgcatatttcagtataacataacttttcttttttacctatatttta
ctaaaaaaatagtaaaagtattgatttttcatgtgaaagggattacaatttcagtgtaaacaaaaagaataaggg
agatacagcct
SEQ ID NO: 5
cagtagttccgataagaacgtaaacgaggatcagagatgatttcagttgcatctgttgttttttcttgcccaatg
agtaggcctatattattacaaccttgttctttaagataacagagtgccgattgtacggagttttcaaaatcagta
gtaacacatggatgtccttgattaagagtatcactatcaacaaagaccagagtcttttttagtctttctagtttc
gcaatttgttcacgactaaattttccgatacagagtacaccaacgacctcctctcctagacttgaaggaatgtcg
ttgaaaaagtgaagcatctcgtagtctagttcataggctcttttttcaataccaagtctaatattataatagtag
atgtcatctaactcgtgttcttcactaacccattgaataattgccacttgatactttttttttttgaattattaa
ttgttttgtatttagtatatccgatttcatcagcgatagttaatatccgatgtctagtatcctctgttacggaaa
gagtttcatctttattaagaacacgtgaaacagttgaaatagatacacctgctaagtttgcgatatctgttaatg
tagccatagtatcctccgcatatttcagtataacataacttttcttttttacctatattttactaaaaaaatagt
aaaagtattgatttttcatgtgaaagggattacaatttcagtgtaaacaaaaagaataagggagatacagcctat
gaatacgttacagttaagagaaaagtttaaagaagtttttggtgtagaagcagatcatactttcttttcaccagg
tcgtattaatttgattggtgagcatacggactacaatggaggtaacgtccttccggtagctattaccctaggtac
ttacggagcggcccgcaaacgtgatgacaaagttttgcgtttcttctcagctaactttgaagagaagggaatcat
cgaagtgccacttgaaaatcttcgttttgaaaaagaacacaactggacaaactatccaaaaggtgttcttcattt
cttgcaagaagctgggcatacgattgattcaggtatggatatttacatctatggtaacattccaaatggatcagg
cttgtcatcatcatcatctttggaattgttgattggtgttattgctgaaaaactttatgaccttaaattggaacg
ccttgacttggttaaaatcggtaagcaaactgaaaatgactttatcggcgttaactctggtatcatggaccaatt
cgctattggtatgggagctgatcaatgtgcgatttacttggatacaaatactctaaagtatgacttgatacccct
tgaccttaaagacaatgttgttgttatcatgaacactaacaaacgtcgtgaattggctgattctaaatacaatga
acgtcgtgctgaatgtgaaac
SEQ ID NO: 6
ctattttgtactgtttcttaatgttaattttgttcctagtttagtaagtgttggtatttttcgaggtgctaagaa
ttgtttattgagaatatccatagctgttcgtcccatttcttctgtatagacagtgacactagaaagtggaggata
cacttgtttagctagagttgtatcgttaaaagagataatttgaatgtcgtcagggaccttgataccattttcctg
aagtgctctgagtgcaccaatagctagactatcactagccgcaaagtaagcatcaggtaaagtagctccactctt
aatcttagaatcaagaagttcatagccagattggacagtgaagtcgccagtcagaataaaaagagggtcatagat
tccctttttcatacagtagttccgataagaacgtaaacgaggatcagagatgatttcagttgcatctgttgtttt
ttcttgcccaatgagtaggcctatattattacaaccttgttctttaagataacagagtgccgattgtacggagtt
ttcaaaatcagtagtaacacatggatgtccttgattaagagtatcactatcaacaaagaccagagtcttttttag
tctttctagtttcgcaatttgttcacgactaaattttccgatacagagtacaccaacgacctcctctcctagact
tgaaggaatgtcgttgaaaaagtgaagcatctcgtagtctagttcataggctcttttttcaataccaagtctaat
attataatagtagatgtcatctaactcgtgttcttcactaacccattgaataattgccacttgatactttttttt
tttgaattattaattgttttgtatttagtatatccgatttcatcagcgatagttaatatccgatgtctagtatcc
tctgttacggaaagagtttcatctttattaagaacacgtgaaacagttgaaatagatacacctgctaagtttgcg
atatctgttaatgtagccatagtatcctccgcatatttcagtataacataacttttcttttttacctatatttta
ctaaaaaaatagtaaaagtattgatttttcatgtgaaagggattacaatttcagtgtaaacaaaaagaataaggg
agatacagcctatgaatacgttacagttaagagaaaagtttaaagaagtttttggtgtagaagcagatcatactt
tcttttcaccaggtcgtattaatttgattggtgagcatacggactacaatggaggtaacgtccttccggtagcta
ttaccctaggtacttacggagcggcccgcaaacgtgatgacaaagttttgcgtttcttctcagctaactttgaag
agaagggaatcatcgaagtgccacttgaaaatcttcgttttgaaaaagaacacaactggacaaactatccaaaag
gtgttcttcatttcttgcaagaagctgggcatacgattgattcaggtatggatatttacatctatggtaacattc
caaatggatcaggcttgtcatcatcatcatctttggaattgttgattggtgttattgctgaaaaactttatgacc
ttaaattggaacgccttgacttggttaaaatcggtaagcaaactgaaaatgactttatcggcgttaactctggta
tcatggaccaattcgctattggtatgggagctgatcaatgtgcgatttacttggatacaaatactctaaagtatg
acttgataccccttgaccttaaagacaatgttgttgttatcatgaacactaacaaacgtcgtgaattggctgatt
ctaaatacaatgaacgtcgtgctgaatgtgaaacagcagtatctgaactccaagaaaaacttgatatccaaacac
ttggtgaattggacctctggacattcgatgcatacagctacttgattaaagataaaaatcgtatcaaacgtgcac
gccatgcagttcttgaaaatcaacgtacacttcaagcccgtaaagctcttgaagcaggagatttggaaggctttg
gtcgtcttatgaatgcttctcacgtatcattggaacatgattacgaagttacaggtcttgaacttgatactttgg
cacacacagcatgggaacaagaaggtgttcttggagctcgtatgacaggagcgggatttggtggatgtgccatcg
cacttgtaaacaaagataaagttgaagatttcaaaaaagcagttggtcaacgttatgaagaagtcgttggttatg
caccaagcttctatatcgctgaagtagctggcggttcacgagtacttgattaatgagaaagaagtagagattatg
gctgaaaatttagtaaacacttttgtaactcaggttattgaaaatagtgattatgaggaattggaccgtatctat
ttgacaaacaaagtttttgctttggttggagaaggagttgctgatgttgaaacagatagctctgaattgattgac
cttaaagaccagttgcttcaagcaggtgttaaagctggttctgtaggtgaacttaatgaagaacaagatatcatc
ggtgctcaactcatggacctgattacaccaagtcctagtgttgtaaatcgtaacttttgggatacctataaatct
aatccagaacaagctatcgctgatttttatgctctcagtaagcgcaatgattatgttaaggtaaaggcaattgct
caaaatatcgcctataagtctccaacaaagtacggtgacttggaaattacgattaacctttcaaaacctgaaaaa
gatccaaaagccatcgctgcagctaaaaatgcggtagcttctgattatccaaaatgccaactttgtatggaaaat
gaaggttatctaggtcgcatcaatcacccagcacgtagtaatcaccgtgttattcgtttccaaatggaagacaag
gagtggggcttccaatattcaccatatgcttactttaatgaacattctattttcttttatggtaagcacgaacca
atgcacatcagtccattgacgtttggtcgtctcctatcaattgttgaagcatttcctggttactttgcaggttca
aatgccgatcttccaattgtaggtggttcaattcttacacacgaacactatcaaggtggtcgccacaccttccca
atggaagtagcaggcattaaagaaaaagttagctttgatggatactctgatgttgaaactggtattgttaattgg
cctatgtcagttcttcgtttaagaagtgaagataaggaaagacttattgctcttgcaaccaaaattttaaattgc
tggcgtggttattcagacgaaaaagctggtgtcttggctgagtctgatggacaacctcaccacaccattacccca
attgctcgtagaaaagacggcaaatttgaattggatttggttcttcgtgacaatcaaacttctgaagaacatcca
gacggtatctaccacccacataaagatgttcaacatattaagaaagaaaacatcggtttgattgaagttatggga
ttggccattcttccacctcgtttgaaaacagaacttaaagatgttgaagattatctattgggtcaaggtaaccaa
gttgctccaattcaccaagaatgggcagatgaactcaaagctcaaaatccgaatgttacggctgaggaagtgaca
gaagttgttcgacaatctgttgcagatatctttgctcgtgtactagaagatgcaggtgtttataagactaatagt
gaaggcttggatcagtttaaagcatttgtagattttgtaaatttagctgattaattgttttttctgaagaaagga
gataagaaatggcaattttagtattaggtggagctggttatatcggttctcacatggttgatcgcttagttgaaa
aaggtcaagaaaaagtagtagtagtagatagtttggtaaccggtcaccgtgctgccgtacatccagatgctattt
tctatcaaggcgatctctctgatcaagattttatgagaaaggtcttcaaagaaaatcctgatgttgatgccgtta
ttcactttgcggcctattcattggttggtgaatcaatggaaaaaccactcaaatattttgataacaatacagctg
gaatggttaaattgcttgaagttatgaacgaatgcggtgttaaatacattgtcttctcatcaacagcagcaactt
acggaattccagaagaaattccaattcttgaaacaacaccacaaaatcctatcaacccatatggtgaaagtaagt
tgatgatggaaaccattatgaagtggtctgataaagcttatggtattaaatatgtgccacttcgttacttcaacg
ttgctggtgcaaaacctgatggatctatcggtgaagaccatggtccagaaactcaccttcttccaattatccttc
aagtagctcaaggtgttcgtgaaaaaatcatgatctttggggatgactacaatactccagacgggactaacgttc
gtgactatgtgcatccattcgacttggcggacgctcacctccttgctgttgaatatcttcgtaaaggtaatgaat
ctacagcctttaacttaggttcatcaacaggtttttcaaaccttcaaattcttgaagcagctcgtaaggtaactg
gtaaagagattccagctgaaaaagttgatcgtcgtcctggtgatcctgatatattgattgcttcatctgaaaaag
cacgtacagttcttggatggaaaccacaatttgataacatcgaaaaaatcatcgcaagtgcttgggcatggcatt
ctagccatccaaaagggtacgatgatcgaggataattaaaataataaacaaaaataaagagtatatataattgtt
gtacttctttgtttttttgaggaaataatatgaaaataagctgcgaaattattggaaaagttgattcaggcgatg
tcagtaaaatttcaatggaaaataataatggtgttgtcatttccacactcacaacaggtgctacacttcaggagt
ttttggttccaacggaaactggtgctcttaaaaatatagtacttggtttcagtgatttcgaggattactataaga
ataatttatgtgcctgccagtccattggtagggttgctggaagaattgggaaagcttcgtatactcataatatgg
ttctttatagccttcctaagaatgagggagaaaactgcttacatggcggtccaaaaggaatgcaggttcaaaatt
ggaactatgtcacaaacctgaatgatgaatatgttgagacaaaatttattagaagactttattcaagtgtagatg
gctttcctggtgatgttacagtctctattagttatagacttgacaataataaccgcttaacaatactctttgaag
ccttcgatgttacagagtcaacagtctttaatccgacaaaccatgtttactttaatcttagcgacaaacaggatt
tatcaagtcatgagttacaaatctattcagactatcgtttagagttggattctgagttaatccctacaggacaaa
aaattaatgtagatggaacaaattatgactttagaaagacaactgacttgctacctcgaattgaagcaaataatg
gttttgatgatgcctttgtagttgaagggggaacttgtgatcatgtgaaagaagtagctattttgcatgacaaag
aaagcggagatggtattgaaatctactcaaacagaaatggtttagtcatttttacgatggatgatatcgaagata
atatttactttgcaagagataaaggcaaaatggcaaaagggcgtgaagctattgctctggaagctcaaacccttc
cagatgccgttaatcacaaaggttttggagatattatcttaaataaaggccatagtgtaaactatgaaattggct
tccaatactttaattcatcaagataa
SEQ ID NO: 7
ctattttgtactgtttcttaatgttaattttgttcctagtttagtaagtgttggtatttttcgaggtgctaataa
ttgtttattgagaatatccatagctgttcgtcccatttcttctgtatagacagtgacactagaaagtggaggata
cacttgtttagctagagttgtatcgttaaaagagataatttgaatgtcgtcagggaccttgataccattttcctg
aagtgctctgagtgcaccaatagctagactatcactagccgcaaagtaagcatcaggtaaagtagctccactctt
aatcttagaatcaagaagttcatagccagattggacagtgaagtcaccagtcagaataaaaagagggtcatagat
tcccttttccatacagtagttccgataagaacgtaaacgaggatcagagatgatttcagttgcatctgttgtttt
ttcttgcccaatgagtaggcctatattattacaaccttgttctttaagataacagagtgccgattgtacggagtt
ttcaaaatcagtagtaacacatggatgtccttgattaagagtatcactatcaacaaagaccagagtcttttttag
tctttctagtttcgcaatttgttcacgactaaattttccgatacagagtacaccaacgacctcctctcctagact
tgaaggaatgtcgttgaaaaagtgaagcatctcgtagtctagttcataggctcttttttcaataccaagtctaat
attataatagtagatgtcatctaactcgtgttcttcactaacccattgaataattgccacttgatacttttcttt
ttttgaattattaattgttttgtatttagtatatccgatttcatcagcgatagttaatatccgatgtctagtatc
ctctgttacggaaagagtttcatctttattaagaacacgtgaaacagttgaaatagatacacctgctaattttgc
gatatctgctaatgtagccatagtatcctcctcatatttcagtataacataacttttatttttttacctatattt
tactaaaaaaatagtaaaaatattgattttccatgtgaaaggggttacgatttcagtataaacaaaaagaataag
tgagatacatcctatgaatacatcacagttaagagaaaagtttaaagaagtttttggtgtagaagcagatcatac
tttcttttcaccaggtcgtattaatttgattggtgagcatacggactacaatggaggtaacgtccttccggtagc
tattaccctaggtacttacggagcggcccgcaaacgtgatgacaaagttttacgtttcttctcagctaactttga
agagaagggaatcatcgaagtgccacttgaaaatcttcgttttgaaaatgaacacaactggacaaactatccaaa
aggtgttcttcatttcttgcaagaagctgggcatacgattgattcaggtatggatatttacatctatggtaacat
tccaaacggatcaggcttgtcatcatcatcatctttggaattattgattggtgttattgttgaaaaactttatga
cattaaattggaacgcctggacttggttaaaatcggaaaacaaacggaaaatgactttattggcgttaactctgg
tatcatggaccaattcgctattggtatgggaactgatcaatgtgcgatttacttggacacaaatactctaaagta
tgacttggtaccccttgacctcaaggataatgtcgtagtcatcatgaacactaacaaacgtcgtgaattggctga
ttctaaatacaatgaacgtcgtgctgaatgtgaaacagcagtatctgaactacaagaaaaattggatatccaaac
tctcggtgaattagacttcttgacatttgacgcatacagctatttgattaaagatgaaaaccgtatcaaacgtgc
acgccatgtagttcttgaaaatcaacgtacacttcaagctcgtaaagctcttgaagcaggagatttggaaggctt
tggacgccttatgaatgcttctcatgtgtcattggaatatgattacgaagttacaggtcttgaacttgatacttt
ggcacacacagcttgggaacaagaaggagtattaggagcccgcatgacaggagctggtttcggtggatgtgccat
tgcacttgtaaacaaagacaaagttgaagacttcaaaaaagcagttggtcaacgctatgaagaagtcgttggtta
tgcaccaagcttctatattgccgaagtaactggtggttcacgagtacttgattaatgagaaagaagtagagatta
tggctgaaaatttagtaaacacttttgtaactcaggttattgaaaatagtgattatgaggaattggaccgtatct
atttgacaaacaaagtttttactttggttggagaaggagttgctgatgttgaaacagatagctctgagttgattg
accttaaagaccagttgcttcaagcaggtgttaaagctggttccgtaggtgaacttaacgaagaacaagatatca
tcggtgctcaactcatggacttaattacaccaagaccgagcgttgtcaatcgtaacttctgggatacttataaat
ctaacccagagcaagcaattgctgacttttatgcacaaagtaaacgaaatgattatgtgaaagtcaaagccattg
cacaaaatattgcttataaagcaccaactaaatacggtgacttggaaattacgattaacctttcaaaacctgaaa
aagatcccaaagccatcgctgcagctaaaaatgcggtagcttctgattatccaaaatgccaactttgtatggaaa
atgaaggttatttgggtcgcattaatcacccagcccgcagcaatcaccgtgttgttcgtttccaaatggaagaca
aggagtggggcttccaatactcgccttatgcctactttaacgaacattctatcttcttttatggtaagcacgaac
caatgcacatcagtccattgacgtttggccgtctcctaacaattgttgaagcattccctggttacttcgcaggtt
caaatgccgatcttccaattgtaggtggttcaattcttacacatgaacactatcaaggtggtcgccataccttcc
caatggaagtagcaggcattaaagaaaaagttagctttgatggttactctgatgttgaggctggcatcgttaatt
ggcctatgtctgttcttcgtctaagaagtgaagacaagggaagacttatcgctcttgcaactaaaatcctaaatt
gctggcgtggttattcagacgaaaaagctggggtcttggctgagtctgatggacaacctcaccacaccattactc
caattgctcgtagaaaagacggcaaatttgaattggatttggttcttcgtgacaatcaaacttctgaagaatatc
cagacggtatctatcacccacataaagatgttcaacatattaagaaagaaaatattggtttgattgaagttatgg
gattggccattcttccacctcgtttgaaaacagaacttaaagatgttgaagattatctattaggtcaaggtaacc
aagttgctccaattcaccaagaatgggcagatgaactcaaagctcaaaatccgaatattacggctgaggaagtga
cagaagttgttcgacaatctgttgcagatatctttgctcgtgtactagaagatgcaggtgtttataagactaata
gtgaaggcttggatcagtttaaagcatttgtagattttgtaaatttagctgattaattgttttttctgaagaaag
gagataaaaaatggcaattttagtattaggtggagctggttatatcggttctcacatggttgatcgcttagttga
aaaaggtcaagaaaaagtagtagtagtagatagtttggtaacaggtcaccgtgctgccgtacatccagatgctat
tttttatcaaggcgatctttctgatcaggattttatgagaaaggtcttcaaagaaaaccctgatgttgatgctgt
tattcactttgcggcctattctttggttggtgaatcaatggaaaaaccactcaaatattttgataacaacacagc
tggaatggttaaattgcttgaggttatgaacgaatgtggtgttaaatacattgtcttctcatcaacagcagcaac
ttacggaattccagaagaaattccaattcttgaaacaacaccacaaaatcctatcaacccatatggtgaaagtaa
gttgatgatggaaaccattatgaagtggtcagaccaagcttacggcatcaagtatgtccctcttcgttactttaa
tgtggcgggtgccaaacctgatggttcgattggtgaggaccacggtccagaaactcaccttctaccgattattct
tcaagtagctcaaggtgttcgtgaaaaaatcatgatctttggggatgactacaatactccagacgggactaacgt
tcgtgactatgtgcatccattcgacttggcggacgctcacctccttgctgttgaatatcttcgtaaaggtaatga
atctacagcctttaacttaggttcatcaacaggtttctcaaaccttcaaattcttgaagcagctcgtaaggtaac
tggtaaagagattccagctgaaaaagctgatcgtcgtcctggtgatcctgatatattgattgcttcatctgaaaa
agcacgtacagttcttggatggaaaccacaatttgataacatcgaaaaaatcatcgcaagtgcttgggcatggca
ttctagccatccaaaagggtacgatgatcgaggataattaaaataataaacaaaaaaacaaagagtatatataat
tgttgtactctttgttttttgaggaaataatatgaaaataagctgcgaaattattggaaaagttgattcaggcga
tgtcagtaaaatttcaatggaaaataataatggtgttgtcatttccacactcacaacaggtgctacacttcagga
gtttttggttccaacggaaactggtgctcttaaaaatatagtacttggtttcagtgatttcgaggattactataa
gaataatttatgtgcctgccagtccattggtagggttgctggaagaattgggaaagcttcgtatactcataatat
ggttctttatagccttcctaagaatgagggagaaaactgcttacatggcggtccaaaaggaatgcaggtccaaaa
ttggaactatgtcacaaacctgaatgatgaatatgttgagacaaaatttattagaagactttattcaagtgtaga
tggctttcctggtgatgtgacagtctctattagttatagacttaacaataataaccgcttaacaatactctttga
agccttcgatgttacagagtcaacaatttttaatccgacaaaccatgtttactttaatcttagcgacaaacagga
tttatcaagtcatgagttacaaatctattcagactatcgtttagagttggattctgagttaattccaacaggaca
aaaaattaatgtagatgaaacgaattatgattttagaaagacaactgacttgctacctcgaattgaagcaaataa
tggttttgatgatgcctttgtagttggagggggaacttgtgatcatgtgaaagaagtagctattttgcatgacaa
agaaagtggagatggtattgaaatcttctcaaacagaaatggtttagtcatttttacgatggatgatatcgaaga
taatatttactttgcaagagataaaggcaaaatggcaaaaaggcgtgaagctattgctatggaagctcaaaccct
tccagatgccgttaatcacaaaggttttggagatattatcttagataaaggtcatagtgtaaactatgaaattgg
cttccaatactttaattcatcaagataaccatgtattagtaaaattttagtaaaaaacactgaaattattgactg
cataaaccaattttcatataatgtaaacgtattcaaataataggaggtttccgaaatggaaaaatctaaaggtca
gatgaagtctcgtttatcctacgcagctggtgcttttggtaacgacgtcttctatgcaaccttgtcaacatactt
tatcatgtttgtgacaactcacttgtttaacacaggtgatccaaagcaaaatagtcactacgtactattaatcac
taacattatctctattttgcgtatcttggaagtatttatcgatccattgatcggtaatatgattgataacactaa
tactaagtatggtaaattcaaaccatgggtagttggtggtggtatcatcagttctatcaccttgttgcttctctt
caccgatttaggtggtttgaataaaacaaatcctttcttgtaccttgtactttttggaattatctaccttataat
ggatgtcttctactcgattaaagatatcggtttctggtcaatgcttcctgccttgtctcttgacagtcacgaacg
tgaaaaaatggcaacttttgcccgtattggttctacgattggtgccaatattgtaggtgttgccatcatgccaat
cgttttgttcttctctatgacgaacagtagtggctctggagataaatctggatggttctggtttgcatttatcgt
tgcactcattggtgtgattacatcaattgctgttggtattggtacacgtgaagttgagtcaaaacttcgtgataa
caatgaaaaaactagccttaaacaagtctttaaagttcttggtaaaaacgaccaattgatgtggctgtctctcgg
atattggttctatggtcttggtattaatacacttaatgctcttcaactttattacttcacatttatccttggtga
ttcagggaaatactcaatcctttacggattgaatacagttgttggtttggtttcagtttcactgttccctagtct
tgctggtaaattcaaccgtaaacgtttgttctatggatgtattgcagtaatgctcggtggtatcggaatctttag
tattgcaggtacttcacttccaatgatcttgactgcagctgaactcttcttcattccacaacctcttgtgttcct
tgttatccttatgattatctctgactcagtagaatatggtcaatggaaattgggacaccgtgatgaatcacttac
tttgtcagttcgtccacttgttgataaacttggtggtgcgatgtcaaactggcttgtttctacaattgccgtagc
tgccggtatgacaacaggtgcctcagcatcaacaattacaacacatcaacagtctatttttaagcttagcatgtt
tggtttcccagcagcagcaatgcttatcggtgccttcattattgctcgtaaaatcactttgactgaagcacgtca
cgctaaaattgttgaagaattggaacatcgctttagcgtagcaacttctgaaaatgaagttaaagctaacgtcgt
atctcttgtaacccctacaactggttatttggttgatctctcaagtgttaatgatgaacactttgcttcaggtag
catgggtaaaggtttcgccattaaacctactgatggagctgtctttgcaccaattagtggtaccattcgtcaaat
tcttcctactcgccatgcagttggtattgaaagtgaagatggtgtcattgttcttatccacgttggcatcggaac
agttaaacttaatggtgaaggattcattagttacgtagaacaaggtgatcgtgttgaagttggacaaaaacttct
tgagttctggtcaccaattattgagaaaaatggtcttgatgacacagtacttgtcactgtaactaattcagaaaa
attcagtgctttccatcttgaacaaaaagttggagaaaaggtagaagctttgtctgaagttattaccttcaaaaa
aggagaataatctatgaacatgactgaaaaaattcaaacttatttaaacgatccaaagattgttagcgttaatac
tgttgatgctcactcagatcataagtattttgaatctcttgaagaattttctgaaggggagatgaagttaagaca
atctcttaatggaaaatggaaaattcactatgctcagaatacaaatcaggttttaaaagacttttataaaacaga
atttgatgaaactgatttgaatttcatcaatgtaccaggtcatttagagcttcaaggttttggttctccacaata
tgtgaatacccaatatccttgggatggtaaagaattccttcgtccacctcaagttcctcaagaatcaaatgctgt
tgcatcatacgttaaacattttaccttgaatgatgcattaaaagataaaaaagtatttatctcattccaaggggt
tgctacttccatctttgtatgggtcaatggtaacttcgtaggatacagtgaagattcatttacacctagtgaatt
tgaaattagtgattaccttgttgaaggtgataacaagttggcggtagctgtttatcgttactctacagcaagctg
gttggaagaccaagacttctggagactttacggtatttttagagatgtttacttgtatgctattccaaaagttca
cgttcaagatctctttgttaagggagattatgattaccaaacaaaagcaggtcaattggatattgatttgaagac
tgttggtgattatgaagacaagaagattaaatatgttctttcagattatgaaggcatcgttacagaaggtgatgc
atctgttaatggtgacggtgaactatctgtaagtcttgaaaatcttaaaatcaaaccttggagtgctgaaagtcc
taaactttacgatttgatccttcatgttttggatgatgaccaagttgttgaagtcgttccagttaaagttggatt
cagacgctttgaaattaaagataaacttatgcttttgaatggtaagagaattgtctttaaaggggttaacagaca
cgaatttaacgctagaacaggacgttgtatcactgaagaagatatgctttgggatatcaaagtgatgaagcaaca
taacatcaatgctgttcgtacttcacactatcctaaccaaacacgttggtatgaattgtgtgatgaatatggact
ttatgttatcgatgaagccaaccttgaaacacacggtacatggcaaaaacttggtctatgcgaaccttcatggaa
tatcccagctagtgaaccagaatggttgcctgcttgtttggatcgtgccaataacatgttccaacgcgataagaa
ccacgctagtgttatcatttggtcttgtggtaatgaatcatatgctggtaaagatattgctgacatggctgatta
cttccgtagtgttgacaatactcgtccagttcactatgaaggtgttgcatggtgtcgtgagtttgattacattac
agacatcgaaagtcgtatgtatgcgaaaccagctgatatcgaagaatacctcacaactggtaaactagttgatct
ttcaagcgttagtgataaacactttgcttcaggtaacctaactaacaaacctcaaaaaccttatatttcatgtga
atacatgcacacaatgggtaactctggtggtggattgcaactctacactgacttagagaaatatccagaatacca
aggtggatttatttgggacttcattgaccaagctatttacaaaacacttccaaatggtagcgaattcctatcata
tggtggtgactggcatgatagaccttctgactacgaattttgtggaaatggtatcgtctttgcagatcgtaccct
aactccaaaacttcaaacagttaaacatctttactctaatattaagattgctgttgatgaaaaatcagtaactat
caagaatgataatctcttcgaagatctttctgcttatactttcctagctagagtttacgaagatggtagaaaagt
tagtgaaagtgaatatcactttgatgttaaaccaggcgaagaagcaacattcccagttaactttgtagtcgaggc
ttcaaattctgaacaaatttacgaagttgcttgtgttctgagggaagcaactgaatgggctcctaaaggtcatga
aattgttcgtggtcaatatgttgttgaaaagattagcactgaaacaccagttaaagcacctttgaatgttgttga
aggcgacttcaacatcggtattcaaggacaaaacttctcaatcttgctttcacgtgcacaaaatactttagtatc
tgctaagtataatggtgttgaattcattgagaaaggtcctaaacttagcttcactcgtgcttacactgacaacga
tcgtggtgctggatatccattcgaaatggcaggctggaaggttgctggaaactatagtaaagttacagatactca
aattcaaatcgaagacgactctgttaaagtgacttatgttcatgaattgccaggcttgtctgatgtcgaagttaa
ggtaacttatcaagttgattacaagggtcgaatctttgttactgcaaactatgatggtaaagcaggtttgccaaa
cttccctgaatttggtctagaatttgctatcggttcacaatttacaaaccttagctattatggatacggtgcaga
agaaagctaccgtgataaacttcctggtgcctatcttggtcgatatgaaacatctgttgaaaagacatttgctcc
atatctaatgccacaagaatctggtaatcactatggtactcgtgaattcacagtatctgatgataaccataatgg
tcttaaattcaccgcacttaataaagcattcgaattcagtgctttgcgtaacagtactgaacaaattgaaaatgc
tcgtcaccaatatgagttgcaagaatctgatgctacatggattaaagttcttgctgctcaaatgggtgtaggtgg
tgacgacacatggggtgctccagttcatgacgaattcttgcttagctcagcagatagctatcaattaagcttcat
gattgaaccactaaattag

### EXEMPLARY EMBODIMENTS

Among the provided embodiments are:
1. A method for generating a Streptococcus thermophilus strain exhibiting a “high galactose utilization” profile, comprising:
   a) providing a Streptococcus thermophilus strain, bearing in its genome a gal-lac gene cluster and having a percentage of consumed galactose at the maximum speed of lactose consumption (VₘₐₓLacₕ) as determined by assay I which is less than 50% and optionally a percentage of consumed galactose at the end of lactose consumption (V₀Lacₕ) determined by assay I which is less than 50%;
   b) modifying the sequence of the gal-lac gene cluster of said Streptococcus thermophilus strain to obtain a gal-lac gene cluster with a different sequence, said different sequence comprising SEQ ID NO:2 or a SEQ ID NO:2 derivative; and
   c) selecting a Streptococcus thermophilus strain obtained in step b) which exhibits a “high galactose utilization” profile defined by a percentage of consumed galactose at the maximum speed of lactose consumption (VₘₐₓLacₕ) as determined by assay I which is at least 50%.
2. The method according to embodiment 1, wherein step b) is selected from the group consisting of modifying the sequence of the gal-lac gene cluster of said Streptococcus thermophilus strain to obtain a gal-lac gene cluster with a different sequence, said different sequence comprising SEQ ID NO:3 or a SEQ ID NO:3 derivative, and modifying the sequence of the gal-lac gene cluster of said Streptococcus thermophilus strain to obtain a gal-lac gene cluster with a different sequence, said different sequence comprising SEQ ID NO:4 or a SEQ ID NO:4 derivative.
3. The method according to embodiment 1, wherein step b) is modifying the sequence of the gal-lac gene cluster of said Streptococcus thermophilus strain to obtain a gal-lac gene cluster with a different sequence, said different sequence comprising SEQ ID NO:5 or a SEQ ID NO:5 derivative.
4. The method according to any one of embodiments 1 to 3, wherein step b) is modifying the sequence of the gal-lac gene cluster of said Streptococcus thermophilus strain to obtain a gal-lac gene cluster with a different sequence, said different sequence comprising SEQ ID NO:6 or a SEQ ID NO:6 derivative, in particular the gal operon of said different sequence consists of the sequence as defined in SEQ ID NO:6 or a SEQ ID NO:6 derivative.
5. The method according to any one of embodiments 1 to 4, wherein step b) is modifying the sequence of the gal-lac gene cluster of said Streptococcus thermophilus strain to obtain a gal-lac gene cluster consisting of the sequence as defined in SEQ ID NO:1 or a SEQ ID NO:1 derivative.
6. The method according to any one of embodiments 1 to 5, wherein said Streptococcus thermophilus strain of step a) is galactose-negative.
7. A Streptococcus thermophilus strain obtainable by the method according to any one of embodiments 1 to 6, provided that the strain is not the Streptococcus thermophilus DSM32823 strain deposited at DSMZ on May 29, 2018.
8. A Streptococcus thermophilus strain characterized in that:
   a) the sequence of its gal-lac gene cluster comprises the sequence as defined in SEQ ID NO:2 or a SEQ ID NO:2 derivative; and
   b) it has a “high galactose utilization” profile defined by a percentage of consumed galactose at the maximum speed of lactose consumption (VₘₐₓLacₕ) as determined by assay I which is at least 50%;
   provided that the strain is not the Streptococcus thermophilus DSM32823 strain deposited at DSMZ on May 29, 2018.
9. The Streptococcus thermophilus strain according to embodiment 8, wherein its gal-lac gene cluster is selected from the group consisting of a gal-lac gene cluster, the sequence of which comprises the sequence as defined in SEQ ID NO:3 or a SEQ ID NO:3 derivative and a gal-lac gene cluster, the sequence of which comprises the sequence as defined in SEQ ID NO:4 or a SEQ ID NO:4 derivative.
10. The Streptococcus thermophilus strain according to embodiment 8, wherein the sequence of its gal-lac gene cluster comprises the sequence as defined in SEQ ID NO:5 or a SEQ ID NO:5 derivative.
11. The Streptococcus thermophilus strain according to any one of embodiments 8 to 10, wherein the sequence of its gal-lac gene cluster comprises the sequence as defined in SEQ ID NO:6 or a SEQ ID NO:6 derivative, in particular the gal operon of its gal-lac gene cluster consists of the sequence as defined in SEQ ID NO:6 or a SEQ ID NO:6 derivative.
12. The Streptococcus thermophilus strain according to any one of embodiments 8 to 11, wherein the sequence of its gal-lac gene cluster consists of the sequence as defined in SEQ ID NO:1 or a SEQ ID NO:1 derivative.
13. The method according to any one of embodiments 1 to 6 or the Streptococcus thermophilus strain according to any one of embodiments 7 to 12, wherein the strain has a “high galactose utilization” profile defined by a percentage of consumed galactose at the maximum speed of lactose consumption (VₘₐₓLacₕ) as determined by assay I which is selected from the group consisting of a percentage of at least 50%, at least 55%, at least 60%, at least at least 65%, at least 70%, at least 75%, at least 80% and at least 85%.
14. The method according to any one of embodiments 1 to 6 or the Streptococcus thermophilus strain according to any one of embodiments 7 to 12, wherein the “high galactose utilization” profile is further defined by a percentage of consumed galactose at the end of lactose consumption (V₀Lacₕ) as determined by assay I which is at least at least 70%, particularly is selected from the group consisting of a percentage of at least 70%, at least 75%, at least 80%, at least 85%, at least 90%, at least 95% and a percentage which is 100%.
15. The method according to any one of embodiments 1 to 6 and 13 to 14 or the Streptococcus thermophilus strain according to any one of embodiments 7 to 14, wherein the strain has a “high galactose utilization” profile defined by a percentage of consumed galactose at the maximum speed of lactose consumption (VₘₐₓLacₕ) as determined by assay I which is selected from the group consisting of a percentage of at least 50%, at least 55%, at least 60%, at least at least 65%, at least 70%, at least 75%, at least 80% and at least 85%, and a percentage of consumed galactose at the end of lactose consumption (V₀Lacₕ) as determined by assay I which is selected from the group consisting of a percentage of at least 70%, at least 75%, at least 80%, at least 85%, at least 90%, at least 95% and a percentage which is 100%.
16. The method according to any one of embodiments 1 to 6 and 13 to 15 or the Streptococcus thermophilus strain according to any one of embodiments 7 to 15, wherein the strain has a “high galactose utilization” profile defined by either a) a percentage of consumed galactose at the maximum speed of lactose consumption (VₘₐₓLacₕ) as determined by assay I which is selected from the group consisting of a percentage of at least 70%, at least 75%, at least 80% and at least 85%, and a percentage of consumed galactose at the end of lactose consumption (V₀Lacₕ) as determined by assay I which is selected from the group consisting of a percentage of at least 80%, at least 85%, at least 90%, at least 95% and a percentage which is 100%, or b) a percentage of consumed galactose at the maximum speed of lactose consumption (VₘₐₓLacₕ) as determined by assay I which is at least 50% and less than 70%, and a percentage of consumed galactose at the end of lactose consumption (V₀Lacₕ) as determined by assay I which is at least 70% and at most 80%.
17. The method according to any one of embodiments 1 to 6 and 13 to 16 or the Streptococcus thermophilus strain according to any one of embodiments 7 to 16, wherein said SEQ ID derivative has at least 97% identity with said SEQ ID.
18. The Streptococcus thermophilus strain according to any one of embodiments 7 to 17, whose genome sequence has an identity which is at most 99.98%, at most 99.97%, at most 99.6% or at most 99.5% to the genome sequence of the DSM32823 strain.
19. The Streptococcus thermophilus strain according to any one of embodiments 7 to 17, further provided that the strain is not a variant of the DSM32823 strain.
20. A culture comprising the Streptococcus thermophilus strain of any one of embodiments 7 to 19, and optionally at least one bacterial strain and/or ingredient(s).
21. A kit-of-part comprising or consisting of a) the Streptococcus thermophilus strain according to any one of embodiments 7 to 19, and b) at least one other bacterial strain and/or ingredient(s).
22. A food or feed product comprising the Streptococcus thermophilus strain of any one of embodiments 7 to 19, the culture of embodiment 20 or the kit-of-part of embodiment 21, in particular a dairy, meat or cereal food or feed product, in particular a fermented dairy food product.
23. A method to manufacture a fermented product, comprising:
   a) inoculating a substrate, in particular a milk substrate, with the Streptococcus thermophilus strain of any one of embodiments 7 to 19, the culture of embodiment 20 or the kit-of-part of embodiment 21; and
   b) fermenting the inoculated substrate obtained from step a) to obtain a fermented product, preferably a fermented dairy product.
24. A method to manufacture pasta-filata cheese, comprising:
   a) providing or producing a curd suitable for stretching, wherein said curd is obtained by inoculating and fermenting milk with the Streptococcus thermophilus strain of any one of embodiments 7 to 19, the culture of embodiment 20 or the kit-of-part of embodiment 21;
   b) stretching the curd of step a) to obtain a stretched curd; and
   c) manipulating the stretched curd of step b), to finally end up with a pasta-filata cheese.
25. The method according to embodiment 24, wherein step a) of producing a curd suitable for stretching comprises:
   a1) inoculating milk with the Streptococcus thermophilus strain of any one of embodiments 7 to 19, the culture of embodiment 20 or the kit-of-part of embodiment 21, and optionally with a milk coagulant;
   a2) fermenting the inoculated milk of step a1) to obtain a coagulated milk;
   a3) cutting the coagulated milk of step a2), heating and stirring, to obtain a mix of curd and whey; and
   a4) draining the mix of curd and whey of step a3), to obtain a curd suitable for stretching.
26. The method according to embodiment 24 or 25, further comprising washing the curd, when heating and stirring at step a3).
27. Use of at least a Streptococcus thermophilus strain of any one of embodiments 7 to 19 to manufacture a pasta-filata cheese.
28. Use of at least a Streptococcus thermophilus strain of any one of embodiments 7 to 19 to produce a cheese whey which has a galactose concentration decreased as compared to a cheese whey produced using a Streptococcus thermophilus strain bearing in its genome a gal-lac gene cluster and having a percentage of consumed galactose at the maximum speed of lactose consumption (VₘₐₓLacₕ) as determined by assay I which is less than 50% and optionally a percentage of consumed galactose at the end of lactose consumption (V₀Lacₕ) determined by assay I which is less than 50%.

### EXAMPLES

### Example 1: Ability of 3 Representative Strains of S. thermophilus to Utilize the Galactose Moiety of Lactose

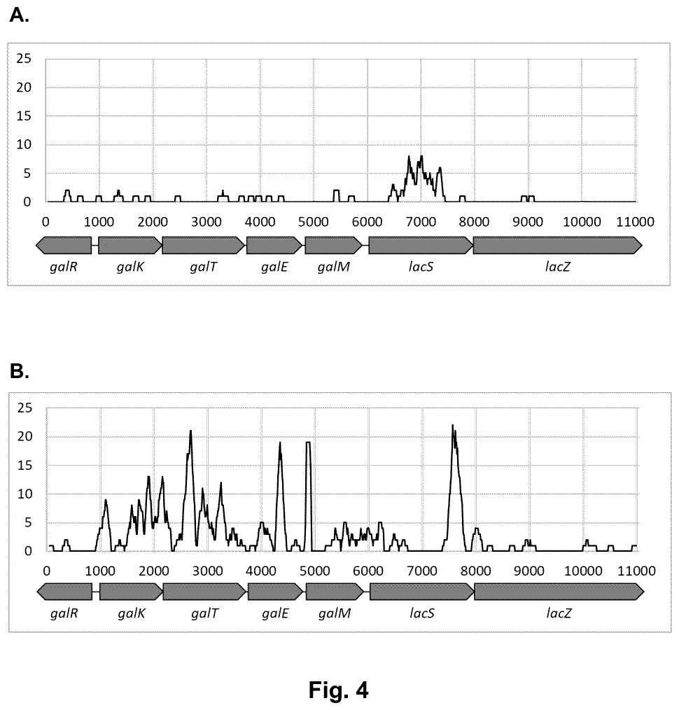

Three representative strains from DuPont collection of strains (DSM32823, DSM33036 and DGCC7773), that have been used industrially for years in dairy fermentation (cheese and fresh fermented milk fermentation), were tested for their utilization of galactose from lactose. Strains were grown in conditions inspired from de Vin et al. (2005). Practically, DSM32823, DSM33036 and DGCC7773 were pre-cultivated twice successively in M17 broth supplemented with lactose 5 g/L for 12 hours at 37° C. The pre-culture was then used to inoculate at 1% (v/v) M17 broth supplemented with lactose 5 g/L (300-ml culture). The culture was then incubated at 37° C. in a water bath. To evaluate the lactose concentration and galactose concentration in the medium over time, every 30 min, a sample of the culture was withdrawn (5 ml), filtered through a 0.2 μm Nylon filter and placed into a 2 ml HPLC vial. Filtered samples were stored at −20° C. until further analysis. Five μL of the sample were injected on an Agilent 1200 HPLC (high-performance-liquid-chromatography). The elution was done in isocratic mode with 0.025N sulfuric acid solution at 0.7 mL/min. Sugars were separated in 20 min onto an H+ ion exchange column (ROA Rezex® 150 mm×7.8 mm×8 μm) and were detected with a refractometer. Quantification was performed comparatively to an external calibration. The calculation of sugar amount was performed with Chromeleon reprocessing software (ThermoFischer Scientific) and the concentration of lactose (mM) and the concentration of galactose (mM) at a given time point is determined. Concentration of galactose below 0.1 g/L (0.5 mM) is considered not measurable. The evolution of concentration over time is represented in FIG. 3.

The results indicated in FIGS. 3A and 3B that both DSM33036 and DGCC7773 behaved similarly, consuming only part of the galactose originating from lactose hydrolysis within 8 hours of growth, and thus are resembling the majority of the strains according to de Vin at al. (2005).

Actually,
in a first phase, corresponding to a slow consumption (hydrolysis) of lactose, the strain consumed the galactose that did not accumulate,
then, in a second phase corresponding to a rapid consumption of lactose, the strain was not capable of consuming all the galactose that was produced from lactose hydrolysis, the galactose accumulated in the medium,
In a third and final phase, when no more lactose was available from the medium, the strains were consuming some but not all the available galactose.

On the contrary, after the first phase, DSM32823 consumed almost all the galactose produced from the lactose, that was finally rapidly consumed to exhaustion in the third phase.

### Example 2: Comparative Genomics of the Gal-Lac Gene Cluster of DSM33036, DSM32823 and DGCC7773

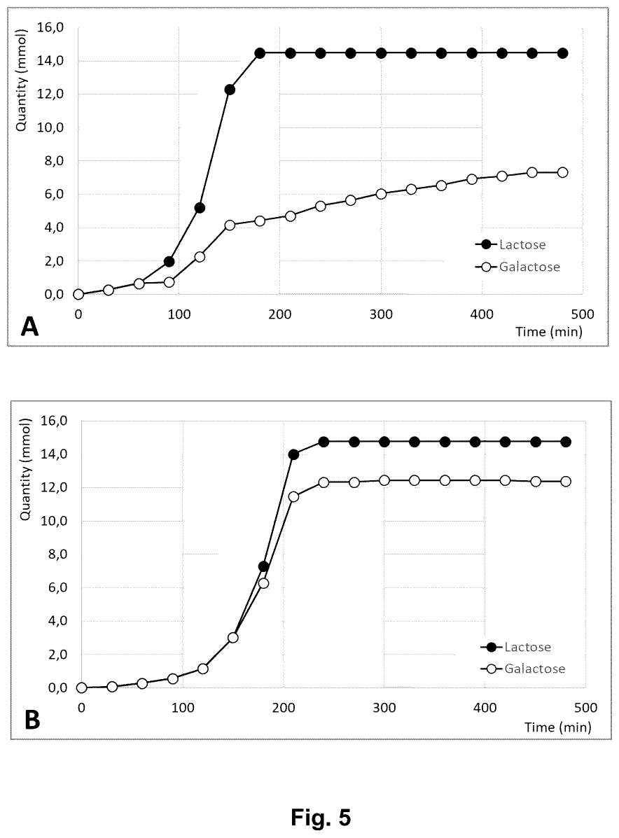

First, the sequence of the gal-lac gene cluster of two strains exhibiting a similar metabolism of galactose (DSM33036 and DGCC7773) were compared and the nucleotide differences between these 2 sequences were analyzed. Thus, following sequence alignment of SEQ ID NO:7 (DSM33036) and SEQ ID NO:8 (DGCC7773), the density of SNPs was calculated by counting the number of variable positions within a 100-bp sliding window which was slid by 10-bp increments. The density of the single-nucleotide polymorphisms (SNPs) between the 2 sequences was aligned with a representation, at scale, of the open reading frames (ORFs) comprised within the gal-lac gene cluster (FIG. 4A).

The sequence identity between the 2 sequences is 99.46%. It is immediately apparent from FIG. 4A that the gal-lac gene cluster of the two strains exhibit low variability over the whole gal-lac gene cluster, with the exception of some minor variabilities throughout the lacS gene.

In view of the difference of metabolism of the galactose (produced from lactose) between the DSM32823 strain, on one hand, and the DSM33036 and DGCC7773 strains, on the other hand, the sequence of the gal-lac gene cluster of DSM32823 (SEQ ID NO:1) and the sequence of the gal-lac gene cluster of DSM33036 (SEQ ID NO:7) were compared, and the nucleotide differences analyzed as described above. The density of the single-nucleotide polymorphisms (SNPs) between the 2 sequences was aligned with a representation, at scale, of the open reading frames (ORFs) comprised within the gal-lac gene cluster (FIG. 4B).

The sequence identity between the 2 sequences is 96.25%. It is immediately apparent from FIG. 4B that some regions are highly variable between strains DSM32823 and DSM33036: the region found between the galR and galK genes, some parts of the coding sequence of the galK gene, some parts of the coding sequence of the galT gene, the middle part of the coding sequence of the galE gene, the region between the galE and the galM genes, and the very end of the lacS gene. Other parts of the gal-lac operon gene cluster, like the galR gene and the lacZ gene, exhibit a low variability.

Altogether these data show that strains exhibiting a similar metabolism of galactose from lactose present little variability between their gal-lac gene clusters, whereas strains exhibiting a different metabolism of galactose from lactose present a significant variability in some regions of the gal-lac gene cluster.

### Example 3: Construction of a Derivative of DSM33036 in which the Gal-Lac Gene Cluster was Replaced by that of DSM32823

To proceed to the replacement of the gal-lac gene cluster of DSM33036 with that of DSM32823, in a first stage, the gal-lac gene cluster was removed from DSM33036 genome. For this purpose, a synthetic DNA, named Ery-D-gal-lac, was produced as an assembly of three genetic elements: i) a genomic sequence from DSM33036 corresponding to the upstream region of the gal-lac gene cluster (downstream from galR), named Down-galR, ii) an erythromycin resistance gene originating from the cloning vector pG+HOST9 (Life Science), named EryR and iii) a genomic sequence from DSM33036 corresponding to the downstream region of the gal-lac gene cluster (downstream from lacZ), named Down-lacZ. Each of the three genetic elements was obtained through PCR amplification. The EryR element was obtained using pG+HOSTt9 plasmid preparation as a template and primers pG9ery-F1 (5′-TGTTCGTGCTGACTTGCAC (SEQ ID NO:9) and Ery-pG9-R3 (5′-CCTCGAGGTCGACGGTATC (SEQ ID NO: 10)). For PCR amplification, the conditions were: 98° C. for 30 sec followed by 33 cycles of 10 sec at 98° C., 30 sec at 58° C. and 45 sec at 72° C., then finally an incubation at 72° C. for 7 min. The enzyme used for amplification was the LA Taq DNA Polymerase (TaKaRa) using the LA-Taq PCR buffer II containing 2.5 mM of MgCl2. The Down-galR element was obtained using DSM33036 genomic DNA as a template. Two mL of an overnight culture of the strain in M17 containing 70 g/L of sucrose were harvested and resuspended in 180 μL of a lysis buffer (lysozyme 20 μg/mL, mutanolysin 120 U/mL, Tris-HCl 20 mM, EDTA 2 mM, Triton-X100 0.12%) and incubated for 1 hour at 37° C.; then the DNeasy Tissue kit (Qiagen, Germany) was used to purify the DNA according to manufacturer's instruction (DNA was resuspended in 100 μL of Tris-HCl 10 mM). PCR amplification was performed as described for the EryR element using primers down-galR_F1 (5′-ATCGTCCAGACAATGGCATG (SEQ ID NO:11)) and GIB-GalR-eryR1 (5′-GTGCAAGTCAGCACGAACACTGAACCATAAACCTGAATAGG (SEQ ID NO:12)) The Down-lacZ element was obtained using DSM33036 genomic DNA as a template and primers GIB-lacZ-eryF1 (5′-GATACCGTCGACCTCGAGGGTACTGATTAGCACTCCAAC (SEQ ID NO:13)) and down-LacZ-R1 (5′-AGATTACCCTGCCTCAATTG (SEQ ID NO:14)). PCR amplification was performed as described for the EryR element. Down-galR and Down-lacZ elements were separately ligated to the EryR element. Ligation was performed using 0.1 pmol of each element and the NEBuilder HiFi DNA Assembly kit (New England Biolabs, Ipswich, MA) at 50° C. for 60 minutes. The final Ery-D-gal-lac synthetic DNA (2,175 pb) was obtained by PCR amplifying an equimolar mix of the two ligation products by using down-galR-F1 and down-LacZ-R1 primers in the same condition as for the previously described PCR and using the following conditions: 98° C. for 30 sec followed by 33 cycles of 30 sec at 98° C., 30 sec at 58° C. and 2.25 min at 72° C., then by a final incubation at 72° C. for 7 min. To obtain the final construction, competent cells of the recipient strain DSM33036 were prepared according to the protocol described by Dandoy et al. (2011). Three hundred microliters of competent cells were transformed with 5 pmol of Ery-D-gal-lac synthetic DNA, in the presence of 1 μM of the inducer peptide ComS17-24 (purity>95%; supplied by Peptide 2.0 (Chantilly, VA)). Following 5 hours of incubation at 37° C., 100 μL of serial dilutions in M17 broth of the transformation suspension were plated on the surface of M17-agar supplemented with 5 g/L sucrose, with 5 μg/mL erythromycin) (Sigma, #E5389), and incubated under anaerobic conditions at 37° C. for 48 h. Erythromycin resistant colonies were picked up and verified for their DNA sequence to ensure a proper excision of the gal-lac gene cluster and its replacement by the erythromycin resistance gene and for their lactose- and galactose-negative phenotype. One of the colonies was selected, propagated and named DSM33036::KOgal-lac.

The gal-lac gene cluster from DSM32823 was then introduced into the genome of DSM33036::KOgal-lac to replace the EryR gene. For this purpose, DSM33036::KOgal-lac was transformed with a DNA fragment from DSM32823 encompassing the gal-lac gene cluster (SEQ ID NO:1). Genomic DNA from the donor strains was prepared as described above. The transformant DNA was obtained through PCR amplification of the gal-lac gene cluster from DSM32823 using the above cited primers down-galR-F1 and down-LacZ-R1, generating a 12.3 kb fragment. The following PCR conditions were applied: 98° C. for 5 min, followed by 33 cycles of 30 sec at 98° C., 30 sec at 58° C. and 13 min at 68° C., then with a final extension at 72° C. for 7 min. Eight hundred microliters of competent cells were transformed with 5.5 pmol of amplicon of the gal-lac gene cluster in the presence of 1 μM of the inducer peptide ComS17-24 (purity>95%; supplied by Peptide 2.0 (Chantilly, VA)). After 5 hours of incubation at 37° C., 100 μL of serial dilutions in M17 broth of the transformation suspension were plated on M17 supplemented with 5 g/L lactose and incubated under anaerobic conditions at 37° C. for 48 h. Colonies capable of growing on lactose were picked up and verified for their erythromycin sensitivity to ensure that the erythromycin resistance gene was effectively removed. Some of the colonies were selected and their gal-lac locus was investigated to ensure a proper replacement by the gal-lac gene cluster from DSM32823. Finally, one of the colonies, named DGCC13139 was selected for further investigation.

Globally, the DGCC13139 construct consists in a strain with the DSM33036 genetic background in which the gal-lac gene cluster is replaced at the same location by the gal-lac gene cluster of DSM32823. To further investigate the ability of DGCC13139 to utilize galactose from lactose, the exact same experimental setting was performed as described in Example 1.

Basically, the kinetic of lactose utilization and of galactose production upon growth at 37° C. of the strain in M17 containing 5 g/L of lactose was investigated through HPLC dosage of carbohydrates. For the purpose of comparison, various kinetic values were determined from the HPLC data. These values are: the quantity of lactose consumed (Lacₕ), the quantity of galactose produced (Galₚ) and the quantity of galactose consumed (Gal_{c}); all sugar quantities (in mmol) are normalized for 1 litre of cultivation medium (whatever the volume of culture medium used for the sugar quantity measurements). The determination of these values was performed as follows:
the quantity of lactose consumed (Lacₕ) represents the difference between the initial amount of lactose, normalized for 1 liter of cultivation medium (lacᵢ) minored by the amount of lactose, normalized for 1 liter of cultivation medium, upon strain growth at a given time point (lacₜ): Lacₕ (mmol)=Lacᵢ (mmol)−Lacₜ (mmol).
upon lactose consumption, lactose is hydrolyzed into glucose and galactose, one mol of lactose generating one mol of each monosaccharide. The quantity of galactose produced at a given time point (Galₚ) is thus equivalent to the quantity of lactose consumed (Lacₕ): Galₚ (mmol)=Lacₕ (mmol)
the quantity of galactose consumed (Gale) represents the difference between the quantity of galactose, normalized for 1 liter of medium, upon strain growth (Galₚ) minored by the quantity of galactose, normalized for 1 liter of cultivation medium, upon strain growth at a given time point (Galₜ): Gal_{c} (mmol)=Galₚ (mmol)−Galₜ (mmol).

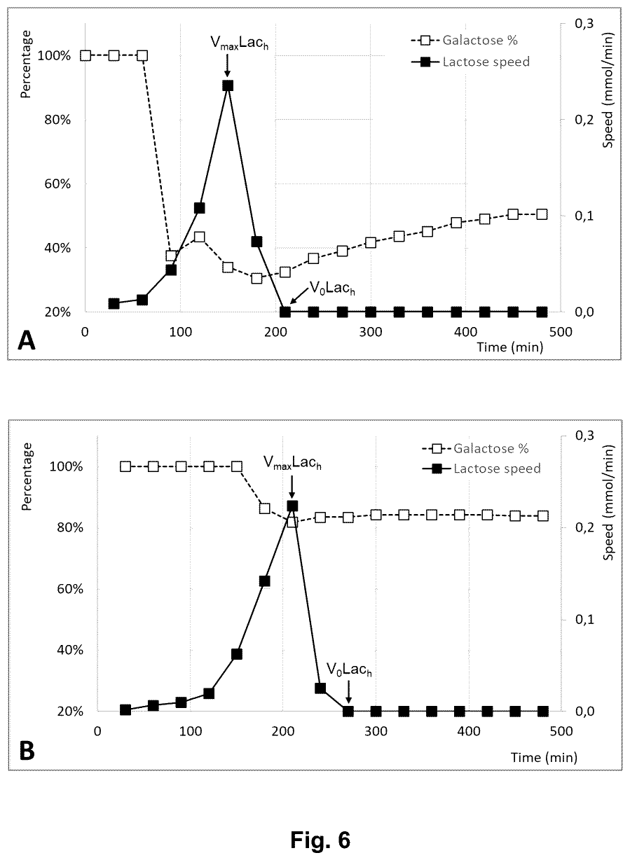

The results on lactose consumption and on galactose consumption are presented in FIG. 5 (A and B). Kinetic of consumption of lactose for both DSM33036 and DGCC13139 are similar (FIG. 5, filled black circles). On the contrary, the kinetic of consumption of the galactose moiety originating from lactose is dramatically changed (FIG. 5, empty circles). Instead of a limited consumption of galactose for DSM33036, DGCC13139 is consuming most of it. From these kinetic values, the proportion of galactose that was consumed could be calculated (for each time point) as the percentage of the galactose produced from lactose that is consumed by the strain during growth: Gal_{c} (%)=[Gal_{c} (mmol)/Galₚ (mmol)]×100. In order to compare results on galactose consumption by the parental and the constructed strains independently from the duration of fermentation, critical “time points” have be identified and selected. The selected “time points” are:
i) that corresponding to the maximum speed of lactose consumption (VₘₐₓLacₕ); and
ii) that corresponding to lactose exhaustion thus to a null speed of lactose consumption (V₀Lacₕ).

The speed of lactose consumption at a given time point is calculated as the difference of lactose quantity between two time points divided by the duration between these two time points and is expressed as VLacₕ (mmol/min)=dLacₕ (mmol)/dt (min). By calculating the speed of lactose consumption at every time point of the kinetics (i.e., every 30 minutes), one can determine at which time point the speed of lactose consumption is maximal (VₘₐₓLacₕ) and at which time point the speed of lactose reaches 0 (V₀Lacₕ), and then calculate the corresponding percentage of galactose consumption (as defined above) for these two time points.

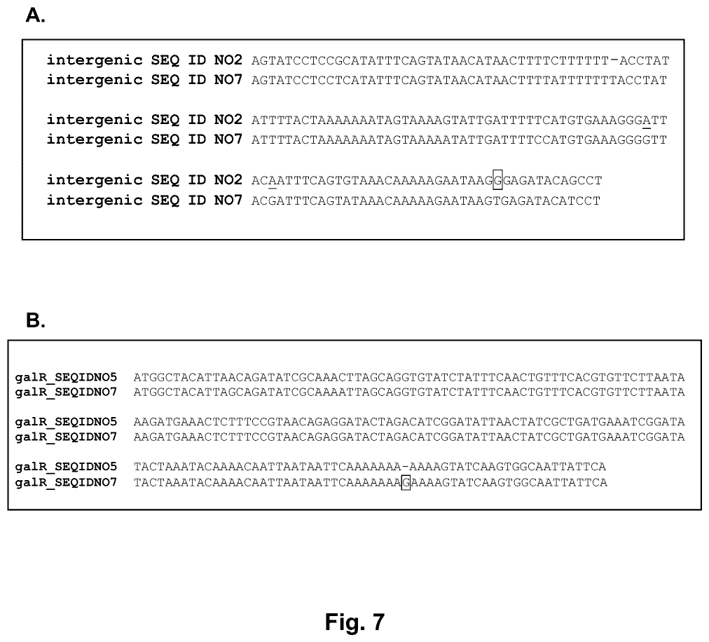

The results of the calculation of speed of lactose consumption (VLacₕ) as well as of the percentage of consumed galactose at each time point, for the parental DSM33036 and the constructed strain DGCC13139, are given in Table 1 and a graphical representation is given in FIG. 6 (A and B).

TABLE 1
Speed of lactose consumption and percentage of
galactose from lactose consumed over time. Values at the
VₘₐₓLacₕ and at the V₀Lacₕ are highlighted in grey

| | DSM33036 | | DGCC13139 | |
|---|---|---|---|---|
| | Speed of lactose | % of | Speed of lactose | % of |
| Time | consumption | consumed | consumption | consumed |
| (min) | (mmol/min) | galactose | (mmol/min) | galactose |
| 0 | | 100% | | 100% |
| 30 | 0.009 | 100% | 0.002 | 100% |
| 60 | 0.013 | 100% | 0.007 | 100% |
| 90 | 0.044 | 38% | 0.010 | 100% |
| 120 | 0.108 | 43% | 0.019 | 100% |
| 150 | 0.236 | 34% | 0.062 | 100% |
| 180 | 0.073 | 31% | 0.142 | 86% |
| 210 | 0.000 | 32% | 0.224 | 82% |
| 240 | 0.000 | 37% | 0.025 | 83% |
| 270 | 0.000 | 39% | 0.000 | 83% |
| 300 | 0.000 | 42% | 0.000 | 84% |
| 330 | 0.000 | 44% | 0.000 | 84% |
| 360 | 0.000 | 45% | 0.000 | 84% |
| 390 | 0.000 | 48% | 0.000 | 84% |
| 420 | 0.000 | 49% | 0.000 | 84% |
| 450 | 0.000 | 50% | 0.000 | 84% |
| 480 | 0.000 | 50% | 0.000 | 84% |

For strain DSM33039, the maximum speed of acidification (VₘₐₓLacₕ) is 0.236 mmol/min, and the null speed of lactose consumption (V₀Lacₕ) is reached 60 minutes after the VₘₐₓLacₕ. For strain DGCC13139, the maximum speed of acidification (VₘₐₓLacₕ) is 0.224 mmol/min, and the null speed of lactose consumption (V₀Lacₕ) is reached 60 minutes after the VₘₐₓLacₕ.

The percentage of consumed galactose at the maximum speed of lactose consumption (VₘₐₓLacₕ) and upon completion of lactose consumption (V₀Lacₕ) is very poor for DSM33036 and are respectively 34% and 32%. On the opposite, DGCC13139 is consuming galactose almost concomitantly to lactose consumption and the percentage of consumed galactose is always above 80%, reaching 82% at the VₘₐₓLacₕ and 83% at the V₀Lacₕ. As a comparison, the percentage of consumed galactose at the VₘₐₓLacₕ and the percentage of consumed galactose at the V₀Lacₕ for the DSM32823 strain (determined in the same conditions as for DSM33036 and DGCC13139) are respectively 79% and 100%.

This experimental work demonstrated that some specific genetic features associated to the gal-lac gene cluster of DSM32823 (SEQ ID NO:1) are responsible for an improved consumption of the galactose moiety of lactose in a medium containing lactose as the sole source of carbohydrate.

These data also show that the percentage of consumed galactose at VₘₐₓLacₕ and the percentage of consumed galactose at V₀Lacₕ are two parameters that can be used to identify strains having a high level of galactose consumption in lactose-containing medium, and to discriminate them from other strains with poor level of galactose consumption. These 2 parameters are calculated using the assay I described below:

### Assay I

The Streptococcus thermophilus strain to be tested is pre-cultivated twice successively in M17 broth supplemented with lactose 5 g/L for 12 hours at 37° C. The pre-culture is then used to inoculate at 1% (v/v) M17 broth supplemented with lactose 5 g/L (300-ml culture). The culture is then incubated at 37° C. in a water bath. Every 30 min, a sample of the culture is withdrawn (5 ml), filtered through a 0.2 μm Nylon filter and placed into a 2 ml HPLC vial. Filtered samples are stored at −20° C. until further analysis. Five μL of the sample are injected on an Agilent 1200 HPLC (high-performance-liquid-chromatography). The elution is done in isocratic mode with 0.025N sulfuric acid solution at 0.7 mL/min. Sugars are separated in 20 min onto an H+ ion exchange column (ROA Rezex® 150 mm×7.8 mm×8 μm) and are detected with a refractometer. Quantification is performed comparatively to an external calibration. The calculation of sugar amount is performed with Chromeleon reprocessing software (ThermoFischer Scientific). From the quantification of lactose and galactose at each time point, the percentage of galactose consumption at the maximum speed of lactose consumption (VₘₐₓLacₕ) and at null speed of lactose consumption (V₀Lacₕ) are determined as follows:
1. for each time point, the quantity of lactose consumed (Lacₕ) is determined by calculating the difference between the initial amount of lactose normalized for 1 liter of cultivation medium (Lacᵢ) minored by the amount of lactose, normalized for 1 liter of cultivation medium, upon strain growth at a given time point (Lacₜ): Lacₕ (mmol)=Lacᵢ (mmol)−Lacₜ (mmol);
2. the quantity of galactose produced at a given time point (Galₚ) is determined as being equivalent to the quantity of lactose consumed Lacₕ: Galₚ (mmol)=Lacₕ (mmol);
3. for each time point, the quantity of galactose consumed (Gal_{c}) is determined by calculating the difference between the quantity of galactose produced, normalized for 1 litre of medium, upon strain growth, at a given time point (Galₚ) minored by the quantity of galactose, normalized for 1 liter of cultivation medium, upon strain growth at a given time point (Galₜ): Gal_{c} (mmol)=Galₚ (mmol)−Galₜ (mmol);
4. for each time point, the proportion of galactose consumed is determined by calculating the percentage of galactose produced from lactose that is consumed by the strain during growth: Gal_{c} (%)=[Gal_{c} (mmol)/Galₚ (mmol)]×100;
5. from the Lacₕ values calculated at each time point, the speed of lactose consumption at a given time point is determined by calculating the difference of lactose quantity between two time points divided by the duration between these two time points: VLacₕ (mmol/min)=dLacₕ (mmol)/dt (min). The time point at which the speed of lactose consumption is maximal (VₘₐₓLacₕ) and the time point at which the speed of lactose consumption reaches 0 (V₀Lacₕ) are selected;
6. the percentage of galactose [Gal_{c} (%)] calculated under 4. corresponding to the time point at which the speed of lactose consumption is maximal (VₘₐₓLacₕ) and the percentage of galactose [Gal_{c} (%)] calculated under 4. corresponding to the time point at which the speed of lactose consumption reaches 0 (V₀Lacₕ) are then respectively considered to be the percentage of galactose consumption at VₘₐₓLacₕ and the percentage of galactose consumption at V₀Lacₕ as determined by assay I for a particular Streptococcus thermophilus strain.

### Example 4: Construction of a Derivative of DGCC7773 in which the Gal-Lac Gene Cluster was Replaced by that of DSM32823

To check that the improved consumption of the galactose moiety of lactose was not dependent on the genetic background of the receiving strain, the gal-lac gene cluster of another strain, DGCC7773, was replaced with that of DSM32823, using the same approach as for EXAMPLE 3. In a first stage, the gal-lac gene cluster was removed from DGCC7773 and replaced by the Ery-D-gal-lac synthetic fragment to generate DGCC7773::KOgal-lac. Then gal-lac gene cluster from DSM32823 was then transferred in the genome of DGCC7773::KOgal-lac to replace the EryR gene. For this purpose, DGCC7773::KOgal-lac was transformed with a DNA fragment from DSM32823 encompassing the gal-lac gene cluster (SEQ ID NO:1). The resulting strain was named DGCC13142.

Globally, DGCC13142 construction consists in a strain with DGCC7773 genetic background the gal-lac gene cluster of which is replaced in the same location by the gal-lac gene cluster of DSM32823 (SEQ ID NO:1). To further investigate the ability of DGCC13142 to consume galactose from lactose, the percentage of consumed galactose at VₘₐₓLacₕ and at V₀Lacₕ. as determined by assay I were calculated and compared with those of DGCC7773. The results are given in Table 2 that provides a synthetic overview of the results.

TABLE 2
Percentage of galactose consumed at the maximum
speed of lactose consumption (VₘₐₓLacₕ) and at
null speed of lactose consumption (V₀Lacₕ)

| | | | % of consumed | |
|---|---|---|---|---|
| | | | galactatose | |
| Strain | Genetic | | at | at |
| Name | background | Genetic transfer | VₘₐₓLacₕ | V₀Lacₕ |
| DSM32823 | WT | Donor strain | 79 | 100 |
| DGCC7773 | WT | Recipient strain | 46 | 36 |
| DGCC13142 | DGCC7773 | Transfer of gal-lac | 79 | 90 |
| | | gene cluster | | |
| | | from DSM32823 | | |

As for DSM33036, the consumption of galactose by DGCC7773 is poor and is of 46% and 36% at the maximum speed of lactose consumption (VₘₐₓLacₕ) and upon completion of lactose consumption (V₀Lacₕ), respectively. Again, on the opposite, DGCC13142 is consuming galactose almost concomitantly to lactose consumption and the percentage of consumed galactose reaches 79% at the VₘₐₓLacₕ and 90% at the V₀Lacₕ.

The additional set of experimental work demonstrated that the specific genetic features associated to the gal-lac gene cluster of DSM32823 (SEQ ID NO:1), that are responsible for an improved consumption of the galactose moiety of lactose, are acting independently to the genetic background of the strain and could thus be broadly applied to a diversity of S. thermophilus strains.

### Example 5: Construction of a Derivative of DSM33036 in which Multiple Fragments of the Gal-Lac Gene Cluster were Replaced by Equivalent Fragments of the Gal-Lac Gene Cluster of DSM32823

Alternatively to the insertion of the gal-lac gene cluster from DSM32823 in place of the native gal-lac gene cluster of a strain which gal-lac gene cluster was preliminary removed from the genome (as done in EXAMPLES 3 and 4), transformation of a wild type strain (possessing a native gal-lac gene cluster) through natural competence with the gal-lac gene cluster from DSM32823 could be envisioned. In such genetic engineering of a strain, a multiplicity of different recombination events may occur leading to multiple possible recombinant strains. Indeed, crossing-over events may happen at each end of the transforming DNA, leading to the complete replacement of the gal-lac gene cluster, or at any places within homologous sequence of the transforming DNA, leading to the replacement of only a portion of the gal-lac gene cluster. Moreover, it is possible that more than two crossing-over events take place, possibly 3 or 4 or even more. Therefore, multiple portions of the gal-lac gene cluster could have been replaced varying in number, length and position.

Competent cells of DSM33036, prepared as described in EXAMPLE 3, were transformed with DNA corresponding to the gal-lac gene cluster from DSM32823 (SEQ ID NO:1). For this purpose, chromosomal DNA from DSM32823 was prepared as described in EXAMPLE 3, and the gal-lac gene cluster was PCR-amplified as described in EXAMPLE 3 using down-galR-F1 and down-LacZ-R1 primers. Transformation was performed by mixing 800 μL of competent cells with 5.3 pmol of PCR amplified DNA in the presence of 1 μM of the inducer peptide ComS17-24. After 5 hours of incubation at 37° C., dilutions in M17 broth of the transformation suspension were plated on M17 supplemented with 5 g/L galactose and incubated under anaerobic conditions at 37° C. for 48 h. Eight colonies growing on galactose were picked up and further cultured (strains DGCC13135, 13136, 13137, 13138, 13392, 13393, 13394 and 13395). The selected strains were then investigated for the percentage of consumed galactose at VₘₐₓLacₕ and at V₀Lacₕ. as determined by assay I.

Results of assay I on these 8 strains are disclosed in Table 3.

TABLE 3
Percentage of the galactose consumed at the maximum
speed of lactose consumption (VₘₐₓLacₕ) and at null
speed of lactose consumption (V₀Lacₕ)

| | | | % age | |
|---|---|---|---|---|
| | | | of consumed | |
| | Genetic | | galactatose at | |
| Strain Name | background | Genetic transfer | VₘₐₓLacₕ | V₀Lacₕ |
| DSM32823 | WT | none | 79 | 100 |
| DSM33036 | WT | none | 34 | 32 |
| DGCC13135 | DSM33036 | fragments of DSM32823 | 84 | 88 |
| | | gal-lac gene cluster | | |
| DGCC13136 | DSM33036 | fragments of DSM32823 | 85 | 94 |
| | | gal-lac gene cluster | | |
| DGCC13137 | DSM33036 | fragments of DSM32823 | 84 | 92 |
| | | gal-lac gene cluster | | |
| DGCC13138 | DSM33036 | fragments of DSM32823 | 88 | 100 |
| | | gal-lac gene cluster | | |
| DGCC13392 | DSM33036 | fragments of DSM32823 | 69 | 78 |
| | | gal-lac gene cluster | | |
| DGCC13393 | DSM33036 | fragments of DSM32823 | 80 | 89 |
| | | gal-lac gene cluster | | |
| DGCC13394 | DSM33036 | fragments of DSM32823 | 64 | 73 |
| | | gal-lac gene cluster | | |
| DGCC13395 | DSM33036 | fragments of DSM32823 | 74 | 80 |
| | | gal-lac gene cluster | | |

Again, consumption of galactose for the selected strains at the maximum speed of lactose consumption (VₘₐₓLacₕ) and upon completion of lactose consumption (V₀Lacₕ) is significantly improved compared to the parental strain and reach values that are close or similar to that of DSM32823 (i.e., the donor strain of the gal-lac gene cluster). Percentage of the galactose consumed at VₘₐₓLacₕ and at V₀Lacₕ ranged respectively from 64 to 88% and from 73 to 100%.

Because various sections of the gal-lac genetic locus may have been affected by the recombination event(s), genomic investigations were performed on the selected strains. A genomic comparison showed that at least one region overlapping from 5′ to 3′, part of the coding sequence of the galR gene, the intergenic region between the galK and galR genes and part of the coding sequence of the galK gene is systematically affected by the recombination events. This region found in all selected strains is defined as SEQ ID NO:5. For every selected strain, a region from the gal-lac gene cluster of DSM32823 encompassing SEQ ID NO:5 has been transferred into the genome of DSM33036. Therefore, SEQ ID NO:5 is believed to be necessary and sufficient to provide the strain with an improved ability to consume galactose from lactose.

This new set of results evidenced that, not all, but only part(s) of, the specific genetic features associated to the gal-lac gene cluster of DSM30823 is (are) responsible and sufficient for an improved consumption of the galactose moiety of lactose. The involvement of only a part of the gal-lac gene cluster of DSM30823 in the improved consumption of the galactose moiety of lactose could have been anticipated, considering that some parts of the gal-lac gene cluster of DSM32823 are not significantly different, even are identical, to their corresponding parts in the gal-lac gene cluster of other strains (see FIG. 4B).

### Example 6: Use of Fragments of the Gal-Lac Gene Cluster Corresponding to SEQ ID NO:5 to Construct Derivative of DSM33036 with Improved Consumption of Galactose from Lactose

Alternatively to the replacement of the whole gal-lac gene cluster of a strain by that from DSM32823, the replacement of only a part of it could possibly be enough to provide an improved consumption of galactose resulting from lactose. This is supported by genetic comparison made in EXAMPLE 2 and by the results obtained in EXAMPLE 5. In particular, EXAMPLE 5 suggested that a section of the gal-lac gene cluster overlapping part of the coding sequence of the galR gene, the intergenic region between the galK and galR genes and part of the coding sequence of the galK gene is of importance for the consumption of galactose. This section is defined as SEQ ID NO:5. Experiments using SEQ ID NO:5 to replace part or all of the equivalent sequence in DSM33036 were investigated.

Competent cells of DSM33036, prepared as described in EXAMPLE 3, were transformed with the DNA fragment from DSM32823 defined in SEQ ID NO:5. For this purpose, genomic DNA from DSM32823 was prepared as described in EXAMPLE 3, and SEQ ID NO:5 amplicon was PCR-generated as described in EXAMPLE 3 using galR-R1 (5′-CAGTAGTTCCGATAAGAACG (SEQ ID NO:15)) and ST89PCR-10Gal-R1 (5′-GTTTCACATTCAGCACGACG (SEQ ID NO:16)) primers. Transformation was performed by mixing 300 μL of competent cells of DSM33036 and 2 pmol of SEQ ID NO:5 amplicon in the presence of 1 μM of the inducer peptide ComS17-24. After 5 hours of incubation at 37° C., dilutions in M17 broth of the transformation suspension were plated on M17 supplemented with 5 g/L galactose and incubated under anaerobic conditions at 37° C. for 48 h. Four colonies growing on galactose were picked up and further cultured (strains DGCC13399, 13400, 13401 and 13402). The selected strains were then investigated for the percentage of consumed galactose at VₘₐₓLacₕ and at the V₀Lacₕ. as determined by assay I. Results of assay I on these 4 selected strains are disclosed in Table 4.

TABLE 4
Percentage of the galactose consumed at the maximum speed of
lactose consumption (VₘₐₓLacₕ) and at null speed of lactose
consumption (V₀Lacₕ) corresponding to lactose exhaustion

| | | | % age of | |
|---|---|---|---|---|
| | | | consumed | |
| | Genetic | | galactatose at | |
| Strain Name | background | Genetic transfer | VₘₐₓLacₕ | V₀Lacₕ |
| DSM32823 | WT | none | 79 | 100 |
| DSM33036 | WT | none | 34 | 32 |
| DGCC13399 | DSM33036 | DNA fragments | 56 | 71 |
| | | corresponding to | | |
| | | SEQ ID n°2 | | |
| DGCC13400 | DSM33036 | DNA fragments | 55 | 73 |
| | | corresponding to | | |
| | | SEQ ID n°2 | | |
| DGCC13401 | DSM33036 | DNA fragments | 58 | 77 |
| | | corresponding to | | |
| | | SEQ ID n°2 | | |
| DGCC13402 | DSM33036 | DNA fragments | 67 | 80 |
| | | corresponding to | | |
| | | SEQ ID n°2 | | |

Again, consumption of galactose for the selected strains at the maximum speed of lactose consumption (VₘₐₓLacₕ) and upon completion of lactose consumption (V₀Lacₕ) is significantly improved compared to the parental strain, reaching values that are close or similar to that of DSM32823 (the donor strain of SEQ ID NO:5). Percentage of the galactose consumed at VₘₐₓLacₕ and at V₀Lacₕ ranged respectively from 55 to 67% and from 71 to 80%.

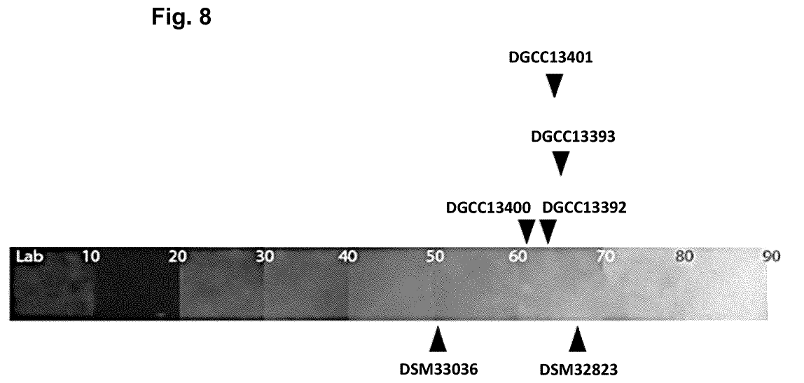

Because recombination events may have occurred in different places of SEQ ID NO:5 when used for the engineering of DSM33036 variants, genomic investigations were performed on the 4 selected strains. A genomic comparison showed that the following particularities specific to the DSM32823 strain (all located within SEQ ID NO:5) have been found in the 4 selected variants whereas they were absent from the DSM33036 strain:
1) the intergenic region between the galR and galK genes bears a G SNP located in the putative Shine-Dalgarno sequence (ribosome binding site) upstream of the galK gene (boxed in FIG. 7A);
2) the intergenic region between the galR and galK genes bears a galK promoter characterized by at least a A nucleotide in positions −9 and −14 (as compared to the first nucleotide of transcription) (underlined positions in FIG. 7A);
3) the beginning of the galR coding sequence, whose length varies among the 4 strains, always contains the deletion of the G nucleotide located at position 175 of the coding sequence of the galR gene within SEQ ID NO:7. The deletion of this G nucleotide leads to a frameshift in the coding sequence of the galR gene and the apparition of a premature STOP codon, leading to a truncated GalR protein (see the alignment of the first 200 nucleotides of the galR coding sequence of DSM33036 and the first 199 nucleotides of the galR coding sequence of DSM32823 in FIG. 7B).

It can therefore be concluded from this comparison that:
the presence of at least the intergenic region between the galR and galK genes, defined as SEQ ID NO:2, containing the particular Shine-Dalgarno sequence upstream of the galK gene (1) and the particular galK promoter (2), is necessary to provide a Streptococcus thermophilus strain exhibiting a “high galactose utilization” profile (improved percentages of galactose consumption at VₘₐₓLacₕ and V₀Lacₕ); and
the presence of a sequence overlapping the beginning of the coding sequence of the galR gene up to the frameshift included, and the intergenic region between the galR and galK genes, sequence defined as SEQ ID NO:3, is necessary and sufficient to design a Streptococcus thermophilus strain exhibiting a “high galactose utilization” profile (improved percentages of galactose consumption at VₘₐₓLacₕ and V₀Lacₕ). SEQ ID NO:3 contains the particular Shine-Dalgarno sequence upstream of the galK gene (1) the particular galK promoter (2) and the particular frameshift in the coding sequence of the galR gene (3).

### Example 7: Production of Pizza Cheese and Evaluation of Functionalities (Residual Lactose and Galactose in Whey and Curd, Browning after Pizza Baking)

Culture to be Tested

The following above-cited strains are tested alone or in combination with Lactococcus lactis and Lactobacillus helveticus strains:
the DSM32823 strain;
the DSM33036 strain;
the DGCC13392 strain;
the DGCC13393 strain;
the DGCC13400 strain;
the DGCC13401 strain;
the DSM32823 strain in combination with Lactococcus lactis strains (commercial product M70; Danisco Dupont reference: 1259559) and a Lactobacillus helveticus strain;
the DSM33036 strain in combination with M70 Lactococcus lactis strains and a Lactobacillus helveticus strain;
the DGCC13392 strain in combination with M70 Lactococcus lactis strains and a Lactobacillus helveticus strain;
the DGCC13393 strain in combination with M70 Lactococcus lactis strains and a Lactobacillus helveticus strain;
the DGCC13400 strain in combination with M70 Lactococcus lactis strains and a Lactobacillus helveticus strain;
the DGCC13401 strain in combination with M70 Lactococcus lactis strains and a Lactobacillus helveticus strain.

Manufacture of a Pizza Cheese

Pizza cheese is manufactured as follows:
cow milk (34.25 g of fat, 34.8 g of protein, 139.2 g of dry matter and 359 mg of urea for 1 L of milk) is heat-treated at 74° C. for 1 minute, and cooled to 35° C.
calcium chloride is added at a concentration of 15 g/100 L of milk
the pH of the milk is standardized at pH 6.4 by CO₂ addition
the milk is distributed in 3-liter VATs
the cultures to be tested (concentration of 1.10¹⁰-1.10¹¹ cfu/g of frozen pellets) are inoculated into the milk in the different VATs
the inoculated milk is fermented at 35° C. for 30 minutes, with inoculation of Marzyme (Danisco Dupont reference 90667) at concentration of 2200 IMC/100 L at t=20 minutes, to obtain a coagulated milk
at t=1 h, the coagulated milk is cut into cubes of 1-1.5 cm sides, and continuously stirred, and then the temperature is increased to 39° C. in 15 minutes
at t=2 h, the curd is molded in mold the diameter of which is 11 cm, and pressed under a weight (1 kg) for 2 minutes; a sample of the whey is removed at this stage to measure the residual sugars
the mix of curd and whey is then drained, by placing the mold without weight at 45° C. until the pH of the curd reached 5.2;
the curd is then cooled to 4° C.;
the curd is then stretched using a cooker stretcher at a rate of 60 rpm with water at 90° C., until the temperature of the stretched curd reaches 55° C.
the stretched curd is brined (in a solution of NaCl at 300 g/L) for 30 minutes
the cheese is then matured and stored during 15 days at 4° C.

Residual Sugars in Whey after Molding

During the manufacture of pizza cheese, a sample of the whey after molding is obtained and the residual lactose and galactose contained thereof are determined as follows: whey sample is diluted in sulfuric acid solution, homogenized into liquid cheese and centrifuged. The supernatant is filtrated and injected on HPLC (high-performance-liquid-chromatography). Sugars are separated onto an H+ ion exchange column (ROA Rezex®) 150 mm×7.8 mm×8 μm) column and detected with refractometer.

The use of the DSM32823, DGCC13392, DGCC13393, DGCC13400 and DGCC13401 strains alone or in combination with Lactococcus lactis and Lactobacillus helveticus strains show an important decrease in the concentration of galactose in the whey as compared to the DSM33036 strain.

Residual Sugars in Curd after Stretching

A sample of the curd during the manufacture of pizza cheese is obtained and the residual lactose and galactose contained thereof are determined. Curd sample is diluted in sulfuric acid solution, homogenized into liquid cheese and centrifuged. The supernatant is filtrated and injected on HPLC (high-performance-liquid-chromatography). Sugars are separated onto an H⁺ ion exchange column (ROA Rezex®) 150 mm×7.8 mm×8 μm) column and detected with refractometer.

The use of the DSM32823, DGCC13392, DGCC13393, DGCC13400 and DGCC13401 strains alone or in combination with Lactococcus lactis and Lactobacillus helveticus strains show an important decrease in the concentration of galactose in the curd as compared to the DSM33036 strain.

Browning and Luminescence Results

Pizzas are prepared as follows: pizza cheese (15-days old cheese matured and stored at 4° C. under foil) is shredded and added on a frozen pizza crust covered of tomato sauce (50 g of pizza cheese is added by quarter of pizza). The pizzas are then baked at 250° C. for 5 min 30 seconds in a Zanolli conveyor pizza oven. For each quarter of pizzas, the browning intensity is calculated. A Minolta colorimeter CR-300 is used to measure the color of the pizza surface after cooking. The CIE L*a*b color space (CIELAB), which expresses color as three numerical values—L* for the lightness, a* for the green-red color component and b* for blue-yellow color component—is used. The L value is used to estimate the browning intensity especially the variation of clearness (the lower the L value, the darker the pizza surface). Thus, a pizza is considered as burnt when the L value is below 55.

The use of the DSM32823, DGCC13392, DGCC13393, DGCC13400 and DGCC13401 strains alone or in combination with Lactococcus lactis and Lactobacillus helveticus strains shows a significant improvement of the browning reduction after pizza baking (increase of the L value) as compared to the use of the DSM33036 strain.

### Example 8: Production and Evaluation of Pizza Cheese Produced Using Streptococcus thermophilus Strains Exhibiting “High Galactose Utilization”

Each of the following strains were tested independently to determine their impact on pizza cheese production and functionalities:
the DSM32823 strain;
the DSM33036 strain;
the DGCC13392 strain;
the DGCC13393 strain;
the DGCC13400 strain;
the DGCC13401 strain.

The manufacture of the pizza cheese was carried out as described in Example 7 above, except that the cheese was matured and stored for 30 days at 4° C.

Residual Sugars in Curd after Stretching

To assess the influence of each strain on the level of residual galactose present in curd after stretching, a sample of the curd during the manufacture of pizza cheese was obtained and residual galactose was quantified as described in Example 7. Table 6 shows the levels of galactose in curd after stretching according to bacterial strain used to manufacture the pizza cheese.

TABLE 6
Galactose levels in curd after stretching according to
bacterial strain used to manufacture the pizza cheese.

| Strain Name | Galactose (g/kg) |
|---|---|
| DSM33036 | 6.9 |
| DSM32823 | 1.7 |
| DGCC13392 | 2.9 |
| DGCC13393 | 2.9 |
| DGCC13400 | 3.4 |
| DGCC13401 | 3.3 |

The use of the DSM32823, DGCC13392, DGCC13393, DGCC13400 and DGCC13401 strains resulted in at least a 2-fold decrease in the concentration of galactose in the curd compared to the DSM33036 reference strain.

These results demonstrate the ability of the strains generated herein exhibiting a “high galactose utilization” profile to effectively and efficiently (e.g., without the need for additional processing time) decrease galactose levels.

Browning and Luminescence Results

Pizzas were prepared as follows: pizza cheese (30-days old cheese matured and stored at 4° C. under foil) was shredded and added on a frozen pizza crust covered with tomato sauce (pizza dough: 30 cm/120 g of cheese). The pizzas were baked at 250° C. for 5 min 30 seconds in a Zanolli conveyor pizza oven. The browning intensity was calculated using a Minolta colorimeter CR-300 to measure the color of the pizza surface after cooking. The CIE L*a*b color space (CIELAB), which expresses color as three numerical values—L* for the lightness, a* for the green-red color component and b* for blue-yellow color component—was used. The L value was used to estimate the browning intensity and variation of clearness (the lower the L value, the darker the pizza surface). For example, a pizza is considered burnt when the L value is below 55.

FIG. 8 shows the L values after pizza baking for each strain tested. Use of DSM32823, DGCC13392, DGCC13393, DGCC13400 and DGCC13401 strains resulted in a decrease in browning after pizza baking (increase of the L value) as compared to the use of the DSM33036 strain.

These results are supportive of the use of Streptococcus thermophilus strains exhibiting “high galactose utilization” to decrease galactose content in pizza cheese and reduce browning of cheese after baking.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. Although the invention may be described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for generating a Streptococcus thermophilus strain exhibiting a high galactose utilization profile, wherein the process comprises introducing a nucleic acid sequence comprising the sequence set forth by SEQ ID NO:2 or a SEQ ID NO:2 derivative to a Streptococcus thermophilus strain bearing in its genome a gal-lac gene cluster and having a percentage of consumed galactose at the maximum speed of lactose consumption (VₘₐₓLacₕ) as determined by assay I which is less than 50.

2-4. (canceled)

5. A Streptococcus thermophilus strain deposited under accession number DSM33851, DSM33852, DSM33853, or DSM33854 on Apr. 21, 2021, at the DSMZ or a mutant thereof.

6. A method according to claim 1, wherein the method comprises:
a) providing a Streptococcus thermophilus strain, bearing in its genome a gal-lac gene cluster and having a percentage of consumed galactose at the maximum speed of lactose consumption (VₘₐₓLacₕ) as determined by assay I which is less than 50%;
b) modifying the sequence of the gal-lac gene cluster of said Streptococcus thermophilus strain to obtain a gal-lac gene cluster with a different sequence, said different sequence comprising SEQ ID NO:2 or a SEQ ID NO:2 derivative; and
c) selecting a Streptococcus thermophilus strain obtained in step b) which exhibits a galactose utilization profile defined by a percentage of consumed galactose at the maximum speed of lactose consumption (VₘₐₓLacₕ) as determined by assay I which is at least 50%.

7. The method according to claim 6, wherein step b) is selected from the group consisting of:
modifying the sequence of the gal-lac gene cluster of said Streptococcus thermophilus strain to obtain a gal-lac gene cluster with a different sequence, said different sequence comprising SEQ ID NO:3 or a SEQ ID NO:3 derivative; and
modifying the sequence of the gal-lac gene cluster of said Streptococcus thermophilus strain to obtain a gal-lac gene cluster with a different sequence, said different sequence comprising SEQ ID NO:4 or a SEQ ID NO:4 derivative.

8-11. (canceled)

12. A Streptococcus thermophilus strain obtainable by the method according to claim 6, provided that the strain is not the Streptococcus thermophilus DSM32823 strain deposited at DSMZ on May 29, 2018.

13. A Streptococcus thermophilus strain, wherein:
a) the sequence of its gal-lac gene cluster comprises the sequence as defined in SEQ ID NO:2 or a SEQ ID NO:2 derivative; and
b) the strain has a galactose utilization profile defined by a percentage of consumed galactose at the maximum speed of lactose consumption (VₘₐₓLacₕ) as determined by assay I which is at least 50%;
provided that the strain is not the Streptococcus thermophilus DSM32823 strain deposited at DSMZ on May 29, 2018.

14. The Streptococcus thermophilus strain according to claim 13, wherein its gal-lac gene cluster is selected from the group consisting of;
a gal-lac gene cluster, the sequence of which comprises the sequence as defined in SEQ ID NO:3 or a SEQ ID NO:3 derivative, and
a gal-lac gene cluster, the sequence of which comprises the sequence as defined in SEQ ID NO:4 or a SEQ ID NO:4 derivative.

15. The Streptococcus thermophilus strain according to claim 14, wherein the sequence of its gal-lac gene cluster comprises the sequence as defined in SEQ ID NO:5 or a SEQ ID NO:5 derivative.

16. The Streptococcus thermophilus strain according to claim 13, wherein the sequence of its gal-lac gene cluster comprises the sequence as defined in SEQ ID NO:6 or a SEQ ID NO:6 derivative, in particular the gal operon of its gal-lac gene cluster consists of the sequence as defined in SEQ ID NO:6 or a SEQ ID NO:6 derivative.

17. The Streptococcus thermophilus strain according to claim 13, wherein the sequence of its gal-lac gene cluster consists of the sequence as defined in SEQ ID NO:1 or a SEQ ID NO:1 derivative.

18. The Streptococcus thermophilus strain according to claim 12, wherein the strain has a galactose utilization profile defined by a percentage of consumed galactose at the maximum speed of lactose consumption (VₘₐₓLacₕ) as determined by assay I which is at least 50%.

19. The Streptococcus thermophilus strain according to claim 12, wherein the galactose utilization profile is further defined by a percentage of consumed galactose at the end of lactose consumption (V₀Lacₕ) as determined by assay I which is at least at least 70%.

20. The Streptococcus thermophilus strain according to claim 12, wherein the strain has a galactose utilization profile defined by:
a percentage of consumed galactose at the maximum speed of lactose consumption (VₘₐₓLacₕ) as determined by assay I which is at least 50%, and
a percentage of consumed galactose at the end of lactose consumption (V₀Lacₕ) as determined by assay I which is at least 70%.

21. The Streptococcus thermophilus strain according to claim 12, wherein the strain has a galactose utilization profile defined by either:
a) both:
a percentage of consumed galactose at the maximum speed of lactose consumption (VₘₐₓLacₕ) as determined by assay I which is at least 70%, and
a percentage of consumed galactose at the end of lactose consumption (V₀Lacₕ) as determined by assay I which is at least 80%; or
b) both:
a percentage of consumed galactose at the maximum speed of lactose consumption (VₘₐₓLacₕ) as determined by assay I which is at least 50% and less than 70%, and
a percentage of consumed galactose at the end of lactose consumption (V₀Lacₕ) as determined by assay I which is at least 70% and at most 80%.

22. The Streptococcus thermophilus strain according to claim 12, wherein said SEQ ID derivative has at least 97% identity with SEQ ID NO:2.

23. The Streptococcus thermophilus strain according to claim 12, wherein genome sequence of the strain has an identity which is at most 99.98% to the genome sequence of the DSM32823 strain.

24-26. (canceled)

27. A culture wherein the culture comprises:
the Streptococcus thermophilus strain of claim 12; and
a bacterial strain from the genus Lactococcus or Lactobacillus.

28. A product, wherein the product:
the product is a dairy, meat, cereal food or feed product; and
the product comprises the Streptococcus thermophilus strain of claim 12.

29. A method to manufacture a fermented product, comprising:
a) inoculating a substrate with the Streptococcus thermophilus strain of claim 12, and
b) fermenting the inoculated substrate obtained from step a) to obtain a fermented product.

30. A method to manufacture pasta-filata cheese, comprising:
a) providing or producing a curd suitable for stretching, wherein said curd is obtained by inoculating and fermenting milk with the Streptococcus thermophilus strain of claim 12,
b) stretching the curd of step a) to obtain a stretched curd, and
c) manipulating the stretched curd of step b) to obtain a pasta-filata cheese.

31-34. (canceled)


SEQUENCE LISTING (17 sequences)

SEQ ID NO: 1 - DNA, 11042 bp, Streptococcus thermophilus
ctattttgtactgtttcttaatgttaattttgttcctagtttagtaagtgttggtatttttcgaggtgctaagaattgtttattgagaatatccatagctgttcgtcccatttcttctgtatagacagtgacactagaaagtggaggatacacttgtttagctagagttgtatcgttaaaagagataatttgaatgtcgtcagggaccttgataccattttcctgaagtgctctgagtgcaccaatagctagactatcactagccgcaaagtaagcatcaggtaaagtagctccactcttaatcttagaatcaagaagttcatagccagattggacagtgaagtcgccagtcagaataaaaagagggtcatagattccctttttcatacagtagttccgataagaacgtaaacgaggatcagagatgatttcagttgcatctgttgttttttcttgcccaatgagtaggcctatattattacaaccttgttctttaagataacagagtgccgattgtacggagttttcaaaatcagtagtaacacatggatgtccttgattaagagtatcactatcaacaaagaccagagtcttttttagtctttctagtttcgcaatttgttcacgactaaattttccgatacagagtacaccaacgacctcctctcctagacttgaaggaatgtcgttgaaaaagtgaagcatctcgtagtctagttcataggctcttttttcaataccaagtctaatattataatagtagatgtcatctaactcgtgttcttcactaacccattgaataattgccacttgatactttttttttttgaattattaattgttttgtatttagtatatccgatttcatcagcgatagttaatatccgatgtctagtatcctctgttacggaaagagtttcatctttattaagaacacgtgaaacagttgaaatagatacacctgctaagtttgcgatatctgttaatgtagccatagtatcctccgcatatttcagtataacataacttttcttttttacctatattttactaaaaaaatagtaaaagtattgatttttcatgtgaaagggattacaatttcagtgtaaacaaaaagaataagggagatacagcctatgaatacgttacagttaagagaaaagtttaaagaagtttttggtgtagaagcagatcatactttcttttcaccaggtcgtattaatttgattggtgagcatacggactacaatggaggtaacgtccttccggtagctattaccctaggtacttacggagcggcccgcaaacgtgatgacaaagttttgcgtttcttctcagctaactttgaagagaagggaatcatcgaagtgccacttgaaaatcttcgttttgaaaaagaacacaactggacaaactatccaaaaggtgttcttcatttcttgcaagaagctgggcatacgattgattcaggtatggatatttacatctatggtaacattccaaatggatcaggcttgtcatcatcatcatctttggaattgttgattggtgttattgctgaaaaactttatgaccttaaattggaacgccttgacttggttaaaatcggtaagcaaactgaaaatgactttatcggcgttaactctggtatcatggaccaattcgctattggtatgggagctgatcaatgtgcgatttacttggatacaaatactctaaagtatgacttgataccccttgaccttaaagacaatgttgttgttatcatgaacactaacaaacgtcgtgaattggctgattctaaatacaatgaacgtcgtgctgaatgtgaaacagcagtatctgaactccaagaaaaacttgatatccaaacacttggtgaattggacctctggacattcgatgcatacagctacttgattaaagataaaaatcgtatcaaacgtgcacgccatgcagttcttgaaaatcaacgtacacttcaagcccgtaaagctcttgaagcaggagatttggaaggctttggtcgtcttatgaatgcttctcacgtatcattggaacatgattacgaagttacaggtcttgaacttgatactttggcacacacagcatgggaacaagaaggtgttcttggagctcgtatgacaggagcgggatttggtggatgtgccatcgcacttgtaaacaaagataaagttgaagatttcaaaaaagcagttggtcaacgttatgaagaagtcgttggttatgcaccaagcttctatatcgctgaagtagctggcggttcacgagtacttgattaatgagaaagaagtagagattatggctgaaaatttagtaaacacttttgtaactcaggttattgaaaatagtgattatgaggaattggaccgtatctatttgacaaacaaagtttttgctttggttggagaaggagttgctgatgttgaaacagatagctctgaattgattgaccttaaagaccagttgcttcaagcaggtgttaaagctggttctgtaggtgaacttaatgaagaacaagatatcatcggtgctcaactcatggacctgattacaccaagtcctagtgttgtaaatcgtaacttttgggatacctataaatctaatccagaacaagctatcgctgatttttatgctctcagtaagcgcaatgattatgttaaggtaaaggcaattgctcaaaatatcgcctataagtctccaacaaagtacggtgacttggaaattacgattaacctttcaaaacctgaaaaagatccaaaagccatcgctgcagctaaaaatgcggtagcttctgattatccaaaatgccaactttgtatggaaaatgaaggttatctaggtcgcatcaatcacccagcacgtagtaatcaccgtgttattcgtttccaaatggaagacaaggagtggggcttccaatattcaccatatgcttactttaatgaacattctattttcttttatggtaagcacgaaccaatgcacatcagtccattgacgtttggtcgtctcctatcaattgttgaagcatttcctggttactttgcaggttcaaatgccgatcttccaattgtaggtggttcaattcttacacacgaacactatcaaggtggtcgccacaccttcccaatggaagtagcaggcattaaagaaaaagttagctttgatggatactctgatgttgaaactggtattgttaattggcctatgtcagttcttcgtttaagaagtgaagataaggaaagacttattgctcttgcaaccaaaattttaaattgctggcgtggttattcagacgaaaaagctggtgtcttggctgagtctgatggacaacctcaccacaccattaccccaattgctcgtagaaaagacggcaaatttgaattggatttggttcttcgtgacaatcaaacttctgaagaacatccagacggtatctaccacccacataaagatgttcaacatattaagaaagaaaacatcggtttgattgaagttatgggattggccattcttccacctcgtttgaaaacagaacttaaagatgttgaagattatctattgggtcaaggtaaccaagttgctccaattcaccaagaatgggcagatgaactcaaagctcaaaatccgaatgttacggctgaggaagtgacagaagttgttcgacaatctgttgcagatatctttgctcgtgtactagaagatgcaggtgtttataagactaatagtgaaggcttggatcagtttaaagcatttgtagattttgtaaatttagctgattaattgttttttctgaagaaaggagataagaaatggcaattttagtattaggtggagctggttatatcggttctcacatggttgatcgcttagttgaaaaaggtcaagaaaaagtagtagtagtagatagtttggtaaccggtcaccgtgctgccgtacatccagatgctattttctatcaaggcgatctctctgatcaagattttatgagaaaggtcttcaaagaaaatcctgatgttgatgccgttattcactttgcggcctattcattggttggtgaatcaatggaaaaaccactcaaatattttgataacaatacagctggaatggttaaattgcttgaagttatgaacgaatgcggtgttaaatacattgtcttctcatcaacagcagcaacttacggaattccagaagaaattccaattcttgaaacaacaccacaaaatcctatcaacccatatggtgaaagtaagttgatgatggaaaccattatgaagtggtctgataaagcttatggtattaaatatgtgccacttcgttacttcaacgttgctggtgcaaaacctgatggatctatcggtgaagaccatggtccagaaactcaccttcttccaattatccttcaagtagctcaaggtgttcgtgaaaaaatcatgatctttggggatgactacaatactccagacgggactaacgttcgtgactatgtgcatccattcgacttggcggacgctcacctccttgctgttgaatatcttcgtaaaggtaatgaatctacagcctttaacttaggttcatcaacaggtttttcaaaccttcaaattcttgaagcagctcgtaaggtaactggtaaagagattccagctgaaaaagttgatcgtcgtcctggtgatcctgatatattgattgcttcatctgaaaaagcacgtacagttcttggatggaaaccacaatttgataacatcgaaaaaatcatcgcaagtgcttgggcatggcattctagccatccaaaagggtacgatgatcgaggataattaaaataataaacaaaaataaagagtatatataattgttgtacttctttgtttttttgaggaaataatatgaaaataagctgcgaaattattggaaaagttgattcaggcgatgtcagtaaaatttcaatggaaaataataatggtgttgtcatttccacactcacaacaggtgctacacttcaggagtttttggttccaacggaaactggtgctcttaaaaatatagtacttggtttcagtgatttcgaggattactataagaataatttatgtgcctgccagtccattggtagggttgctggaagaattgggaaagcttcgtatactcataatatggttctttatagccttcctaagaatgagggagaaaactgcttacatggcggtccaaaaggaatgcaggttcaaaattggaactatgtcacaaacctgaatgatgaatatgttgagacaaaatttattagaagactttattcaagtgtagatggctttcctggtgatgttacagtctctattagttatagacttgacaataataaccgcttaacaatactctttgaagccttcgatgttacagagtcaacagtctttaatccgacaaaccatgtttactttaatcttagcgacaaacaggatttatcaagtcatgagttacaaatctattcagactatcgtttagagttggattctgagttaatccctacaggacaaaaaattaatgtagatggaacaaattatgactttagaaagacaactgacttgctacctcgaattgaagcaaataatggttttgatgatgcctttgtagttgaagggggaacttgtgatcatgtgaaagaagtagctattttgcatgacaaagaaagcggagatggtattgaaatctactcaaacagaaatggtttagtcatttttacgatggatgatatcgaagataatatttactttgcaagagataaaggcaaaatggcaaaagggcgtgaagctattgctctggaagctcaaacccttccagatgccgttaatcacaaaggttttggagatattatcttaaataaaggccatagtgtaaactatgaaattggcttccaatactttaattcatcaagataaccatgtattagtaaaagtttagtaaaaaacactgaaattattgactgtataaaccaatttttatataatgtaaacgcattcaaataataggaggtttccgaaatggaaaaatctaaaggtcagatgaagtctcgtttatcctacgcagctggtgctttcggtaacgacgtcttctatgcaaccatgtcaacatactttatcatgtttgtgacaactcacttgtttaacacaggtgatccaaagcaagatagtcactacgtactattaattactaacattatcgctgctttgcgtatcttggaagtatttatcgatccattgatcggtaatatgattgataacactaatactaagtatggtaaattcaaaccatgggtagttggtggtggtatcatcagttctatcaccttgttgcttctcttcaccgatttaggtggtttgaataaaacaaatcctttcttgtaccttgtactttttggaattatctaccttgtaatggatgtcttctactcgattaaagatatcggtttctggtcaatgattcctgccttgtctcttgatagtcacgaacgtgaaaaaatggcaacttttgcccgtattggttctacgattggtgccaatattgtaggtgttgccatcatgccaatcgttttgttcttctctatgacgaacaatagtggctctggagataaatctggatggttctggtttgcatttatcgttgctctcattggtgtgattacatcaattgctgttggtattggtacacgtgaagttgagtcaaaacttcgtgataacaatgaaaaaactagccttaaacaagtctttaaagttcttggtaaaaacgaccaattgatgtggctgtctctcggatattggttctatggtcttggtattaatacacttaatgctcttcaactttattacttcacatttatccttggtgattcagggaaatactcaatcctttacggattgaatacagttgttggtttggtttcagtttcactgttccctagtcttgctggtaaattcaaccgtaaacgtttgttctatggatgtattgcagtaatgctcggtggtatcggaatctttagtattgcaggtacttcacttccaatgatcttgactgcagctgaactcttcttcattccacaacctcttgtgttccttgttatccttatgattatctctgactcagtagaatatggtcaatggaaattgggacaccgtgatgaatcacttactttgtcagttcgtccacttgttgataaacttggtggtgcgatgtcaaactggcttgtttctacaattgccgtagctgccggtatgacaacaggtgcctcagcatcaacaattacaacacatcaacagtctatttttaagcttagcatgtttggtttcccagcagcagcaatgcttatcggtgccttcattattgctcgtaaaatcactttgactgaagcacgtcacgctgaaattgttgaagaattggaaaatcgctttagcgtagcaacttctgaaaatgaagttaaagctaacgtcgtatctcttgtagcccctacaactggtaaactagttgatctttcaagcgttagtgataaacgctttgcttcaggtagcctaggtaaaggatttgcaatcaaaccttcagatggtaaggtttttgccccaattagtggtacagttcgtcagattctacctactcgtcatgcgattggtattgaaagtgatgatggtgtcgttgttctcgtacacgttggtattggaacagttaaacttaaaggtgaagggttcattagttacgtaaaacaaggtgatcgtgttgaagttggacaaaaacttcttgagttctggtcaccaattattgagaaaaatggtcttgatgacacagtacttgtcactgtaactaattcagaaaaattcagtgctttccatcttgaacaaaaagttggagaaaaggtagaagctttgtctgaagttattaccttcaaaaaaggagaataatttatgaacacgactgaaaaaattcaaacttacttaaacgatccaaagattgttagcgttaatactgttgacgctcactcagatcataagtattttgaatctcttgcagaattttctgagggggagatgaagttaagacaatctcttaatggaaaatggaaaattcactatgctcagaatacaaatcaggttttaaaagacttttataaaacagaatttgatgaaactgatttgaatttcatcaatgtaccaggtcatttagagcttcaaggttttggttctccacaatatgtgaatacccagtatccttgggatggtaaagaattccttcgtccacctcaagttcctcaagaatcaaatgctgttgcatcatacgttaaacattttaccttgaatgatgcattaaaagataaaaaagtatttatctcattccaaggggttgctacttccatctttgtatgggtcaatggtaacttcgtaggatacagtgaagattcatttacacctagtgaatttgaaattagtgattaccttgttgaaggtgataacaagttggcggtagctgtttatcgttactctacagcaagctggttggaagaccaagacttctggagactttacggtatttttagagatgtttacttgtatgctattccaaaagttcacgttcaagatctctttgttaagggagattatgattaccaaacaaaaacaggtcaattggatattgatttgaagactgttggtgattatgaagacaagaagattaaatatgttctttcagattatgaaggcatcgttacagaaggtgatgcatctgttaatggtgacggtgaactatctgtaagtcttgaaaatcttaaaatcaaaccttggagtgctgaaagtcctaaactttacgatttgatccttcatgttttggatgatgaccaagttgttgaagtcgttccagttaaagttggatttagacgctttgaaattaaagataaacttatgcttttgaatggtaagagaattatctttaaaggggttaacagacacgaatttaacgctagaacaggacgttgtatcactgaagaagatatgctttgggatatcaaagtgatgaaacaacataacatcaatgctgttcgtacttcacactatcctaaccaaacacgttggtatgaattgtgtgatgaatatggactttatgttatcgatgaagccaaccttgaaacacacggtacatggcaaaaacttggtctatgcgaaccttcatggaatatcccagctagtgaaccagaatggttgcctgcttgtttggatcgtgccaataacatgttccaacgcgataagaaccacgctagtgttatcatttggtcttgtggtaatgaatcatatgctggtaaagatattgctgacatggctgattacttccgtagtgttgacaatactcgtccagttcactatgaaggtgttgcatggtgtcgtgagtttgattacattacagacatcgaaagtcgtatgtatgcgaaaccagctgatatcgaagaatacctcacaactggtaaactagttgatctttcaagcgttagtgataaacactttgcttcaggtaacctaactaacaaacctcaaaaaccttatatttcatgtgaatacatgcacacaatgggtaactctggtggtggattgcaactctacactgacttagagaaatatccagaataccaaggtggatttatttgggacttcattgaccaagctatttacaaaacacttccaaatggtagcgaattcctatcatatggtggtgactggcatgatagaccttctgactacgaattttgtggaaatggtatcgtctttgcagatcgtaccctaactccaaaacttcaaacagttaaacatctttactctaatattaagattgctgttgatgaaaaatcagtaactatcaagaatgataatctcttcgaagatctttctgcttatactttcctagctagagtttacgaagatggtagaaaagttagtgaaagtgaatatcactttgatgttaaaccaggcgaagaagcaacattcccagttaactttgtagttgaggcttcaaattctgaacaaatttacgaagttgcttgtgttctgagggaagcaactaaatgggctcctaaaggtcatgaaattgttcgtggtcaatatgttgttgaaaagattagcactgaaacaccagttaaagcacctttgaatgttgttgaaggggacttcaacatcggtattcaaggacaaaacttctcaatcttgctttcacgtgcacaaaatactttagtatctgctaagtataatggtgttgaattcattgagaaaggtcctaaacttagcttcactcgtgcttacactgacaacgatcgtggtgctggatatccattcgaaatggcaggctggaaggttgctggaaactatagtaaagttacagatactcaaattcaaatcgaagacgactctgttaaagtgacttatgttcatgaattgccaggcttgtctgatgtcgaagttaaggtaacttatcaagttgactacaagggtcgaatctttgttactgcaaactatgatggtaaagcaggtttgccaaacttccctgaatttggtctagaatttgctatcggttcacaatttacaaaccttagctattatggatacggtgcagaagaaagctaccgtgataaacttcctggtgcctatcttggtcgatatgaaacatctgttgaaaagacatttgctccatatctaatgccacaagaatctggtaatcactatggtactcgtgaattcacagtatctgatgataaccataatggtcttaaattcaccgcacttaataaagcattcgaattcagtgctttgcgtaacagtactgaacaaattgaaaatgctcgtcaccaatatgagttgcaagaatctgatgctacatggattaaagttcttgctgctcaaatgggtgtaggtggtgacgactcatggggtgctccagttcatgacgaattcttgcttagctcagcagatagctatcaattaagcttcatgattgaaccactaaattag

SEQ ID NO: 2 - DNA, 141 bp, Streptococcus thermophilus
agtatcctccgcatatttcagtataacataacttttcttttttacctatattttactaaaaaaatagtaaaagtattgatttttcatgtgaaagggattacaatttcagtgtaaacaaaaagaataagggagatacagcct

SEQ ID NO: 3 - DNA, 335 bp, Streptococcus thermophilus
aattgccacttgatactttttttttttgaattattaattgttttgtatttagtatatccgatttcatcagcgatagttaatatccgatgtctagtatcctctgttacggaaagagtttcatctttattaagaacacgtgaaacagttgaaatagatacacctgctaagtttgcgatatctgttaatgtagccatagtatcctccgcatatttcagtataacataacttttcttttttacctatattttactaaaaaaatagtaaaagtattgatttttcatgtgaaagggattacaatttcagtgtaaacaaaaagaataagggagatacagcct

SEQ ID NO: 4 - DNA, 1136 bp, Streptococcus thermophilus
ctattttgtactgtttcttaatgttaattttgttcctagtttagtaagtgttggtatttttcgaggtgctaagaattgtttattgagaatatccatagctgttcgtcccatttcttctgtatagacagtgacactagaaagtggaggatacacttgtttagctagagttgtatcgttaaaagagataatttgaatgtcgtcagggaccttgataccattttcctgaagtgctctgagtgcaccaatagctagactatcactagccgcaaagtaagcatcaggtaaagtagctccactcttaatcttagaatcaagaagttcatagccagattggacagtgaagtcgccagtcagaataaaaagagggtcatagattccctttttcatacagtagttccgataagaacgtaaacgaggatcagagatgatttcagttgcatctgttgttttttcttgcccaatgagtaggcctatattattacaaccttgttctttaagataacagagtgccgattgtacggagttttcaaaatcagtagtaacacatggatgtccttgattaagagtatcactatcaacaaagaccagagtcttttttagtctttctagtttcgcaatttgttcacgactaaattttccgatacagagtacaccaacgacctcctctcctagacttgaaggaatgtcgttgaaaaagtgaagcatctcgtagtctagttcataggctcttttttcaataccaagtctaatattataatagtagatgtcatctaactcgtgttcttcactaacccattgaataattgccacttgatactttttttttttgaattattaattgttttgtatttagtatatccgatttcatcagcgatagttaatatccgatgtctagtatcctctgttacggaaagagtttcatctttattaagaacacgtgaaacagttgaaatagatacacctgctaagtttgcgatatctgttaatgtagccatagtatcctccgcatatttcagtataacataacttttcttttttacctatattttactaaaaaaatagtaaaagtattgatttttcatgtgaaagggattacaatttcagtgtaaacaaaaagaataagggagatacagcct

SEQ ID NO: 5 - DNA, 1446 bp, Streptococcus thermophilus
cagtagttccgataagaacgtaaacgaggatcagagatgatttcagttgcatctgttgttttttcttgcccaatgagtaggcctatattattacaaccttgttctttaagataacagagtgccgattgtacggagttttcaaaatcagtagtaacacatggatgtccttgattaagagtatcactatcaacaaagaccagagtcttttttagtctttctagtttcgcaatttgttcacgactaaattttccgatacagagtacaccaacgacctcctctcctagacttgaaggaatgtcgttgaaaaagtgaagcatctcgtagtctagttcataggctcttttttcaataccaagtctaatattataatagtagatgtcatctaactcgtgttcttcactaacccattgaataattgccacttgatactttttttttttgaattattaattgttttgtatttagtatatccgatttcatcagcgatagttaatatccgatgtctagtatcctctgttacggaaagagtttcatctttattaagaacacgtgaaacagttgaaatagatacacctgctaagtttgcgatatctgttaatgtagccatagtatcctccgcatatttcagtataacataacttttcttttttacctatattttactaaaaaaatagtaaaagtattgatttttcatgtgaaagggattacaatttcagtgtaaacaaaaagaataagggagatacagcctatgaatacgttacagttaagagaaaagtttaaagaagtttttggtgtagaagcagatcatactttcttttcaccaggtcgtattaatttgattggtgagcatacggactacaatggaggtaacgtccttccggtagctattaccctaggtacttacggagcggcccgcaaacgtgatgacaaagttttgcgtttcttctcagctaactttgaagagaagggaatcatcgaagtgccacttgaaaatcttcgttttgaaaaagaacacaactggacaaactatccaaaaggtgttcttcatttcttgcaagaagctgggcatacgattgattcaggtatggatatttacatctatggtaacattccaaatggatcaggcttgtcatcatcatcatctttggaattgttgattggtgttattgctgaaaaactttatgaccttaaattggaacgccttgacttggttaaaatcggtaagcaaactgaaaatgactttatcggcgttaactctggtatcatggaccaattcgctattggtatgggagctgatcaatgtgcgatttacttggatacaaatactctaaagtatgacttgataccccttgaccttaaagacaatgttgttgttatcatgaacactaacaaacgtcgtgaattggctgattctaaatacaatgaacgtcgtgctgaatgtgaaac

SEQ ID NO: 6 - DNA, 5951 bp, Streptococcus thermophilus
ctattttgtactgtttcttaatgttaattttgttcctagtttagtaagtgttggtatttttcgaggtgctaagaattgtttattgagaatatccatagctgttcgtcccatttcttctgtatagacagtgacactagaaagtggaggatacacttgtttagctagagttgtatcgttaaaagagataatttgaatgtcgtcagggaccttgataccattttcctgaagtgctctgagtgcaccaatagctagactatcactagccgcaaagtaagcatcaggtaaagtagctccactcttaatcttagaatcaagaagttcatagccagattggacagtgaagtcgccagtcagaataaaaagagggtcatagattccctttttcatacagtagttccgataagaacgtaaacgaggatcagagatgatttcagttgcatctgttgttttttcttgcccaatgagtaggcctatattattacaaccttgttctttaagataacagagtgccgattgtacggagttttcaaaatcagtagtaacacatggatgtccttgattaagagtatcactatcaacaaagaccagagtcttttttagtctttctagtttcgcaatttgttcacgactaaattttccgatacagagtacaccaacgacctcctctcctagacttgaaggaatgtcgttgaaaaagtgaagcatctcgtagtctagttcataggctcttttttcaataccaagtctaatattataatagtagatgtcatctaactcgtgttcttcactaacccattgaataattgccacttgatactttttttttttgaattattaattgttttgtatttagtatatccgatttcatcagcgatagttaatatccgatgtctagtatcctctgttacggaaagagtttcatctttattaagaacacgtgaaacagttgaaatagatacacctgctaagtttgcgatatctgttaatgtagccatagtatcctccgcatatttcagtataacataacttttcttttttacctatattttactaaaaaaatagtaaaagtattgatttttcatgtgaaagggattacaatttcagtgtaaacaaaaagaataagggagatacagcctatgaatacgttacagttaagagaaaagtttaaagaagtttttggtgtagaagcagatcatactttcttttcaccaggtcgtattaatttgattggtgagcatacggactacaatggaggtaacgtccttccggtagctattaccctaggtacttacggagcggcccgcaaacgtgatgacaaagttttgcgtttcttctcagctaactttgaagagaagggaatcatcgaagtgccacttgaaaatcttcgttttgaaaaagaacacaactggacaaactatccaaaaggtgttcttcatttcttgcaagaagctgggcatacgattgattcaggtatggatatttacatctatggtaacattccaaatggatcaggcttgtcatcatcatcatctttggaattgttgattggtgttattgctgaaaaactttatgaccttaaattggaacgccttgacttggttaaaatcggtaagcaaactgaaaatgactttatcggcgttaactctggtatcatggaccaattcgctattggtatgggagctgatcaatgtgcgatttacttggatacaaatactctaaagtatgacttgataccccttgaccttaaagacaatgttgttgttatcatgaacactaacaaacgtcgtgaattggctgattctaaatacaatgaacgtcgtgctgaatgtgaaacagcagtatctgaactccaagaaaaacttgatatccaaacacttggtgaattggacctctggacattcgatgcatacagctacttgattaaagataaaaatcgtatcaaacgtgcacgccatgcagttcttgaaaatcaacgtacacttcaagcccgtaaagctcttgaagcaggagatttggaaggctttggtcgtcttatgaatgcttctcacgtatcattggaacatgattacgaagttacaggtcttgaacttgatactttggcacacacagcatgggaacaagaaggtgttcttggagctcgtatgacaggagcgggatttggtggatgtgccatcgcacttgtaaacaaagataaagttgaagatttcaaaaaagcagttggtcaacgttatgaagaagtcgttggttatgcaccaagcttctatatcgctgaagtagctggcggttcacgagtacttgattaatgagaaagaagtagagattatggctgaaaatttagtaaacacttttgtaactcaggttattgaaaatagtgattatgaggaattggaccgtatctatttgacaaacaaagtttttgctttggttggagaaggagttgctgatgttgaaacagatagctctgaattgattgaccttaaagaccagttgcttcaagcaggtgttaaagctggttctgtaggtgaacttaatgaagaacaagatatcatcggtgctcaactcatggacctgattacaccaagtcctagtgttgtaaatcgtaacttttgggatacctataaatctaatccagaacaagctatcgctgatttttatgctctcagtaagcgcaatgattatgttaaggtaaaggcaattgctcaaaatatcgcctataagtctccaacaaagtacggtgacttggaaattacgattaacctttcaaaacctgaaaaagatccaaaagccatcgctgcagctaaaaatgcggtagcttctgattatccaaaatgccaactttgtatggaaaatgaaggttatctaggtcgcatcaatcacccagcacgtagtaatcaccgtgttattcgtttccaaatggaagacaaggagtggggcttccaatattcaccatatgcttactttaatgaacattctattttcttttatggtaagcacgaaccaatgcacatcagtccattgacgtttggtcgtctcctatcaattgttgaagcatttcctggttactttgcaggttcaaatgccgatcttccaattgtaggtggttcaattcttacacacgaacactatcaaggtggtcgccacaccttcccaatggaagtagcaggcattaaagaaaaagttagctttgatggatactctgatgttgaaactggtattgttaattggcctatgtcagttcttcgtttaagaagtgaagataaggaaagacttattgctcttgcaaccaaaattttaaattgctggcgtggttattcagacgaaaaagctggtgtcttggctgagtctgatggacaacctcaccacaccattaccccaattgctcgtagaaaagacggcaaatttgaattggatttggttcttcgtgacaatcaaacttctgaagaacatccagacggtatctaccacccacataaagatgttcaacatattaagaaagaaaacatcggtttgattgaagttatgggattggccattcttccacctcgtttgaaaacagaacttaaagatgttgaagattatctattgggtcaaggtaaccaagttgctccaattcaccaagaatgggcagatgaactcaaagctcaaaatccgaatgttacggctgaggaagtgacagaagttgttcgacaatctgttgcagatatctttgctcgtgtactagaagatgcaggtgtttataagactaatagtgaaggcttggatcagtttaaagcatttgtagattttgtaaatttagctgattaattgttttttctgaagaaaggagataagaaatggcaattttagtattaggtggagctggttatatcggttctcacatggttgatcgcttagttgaaaaaggtcaagaaaaagtagtagtagtagatagtttggtaaccggtcaccgtgctgccgtacatccagatgctattttctatcaaggcgatctctctgatcaagattttatgagaaaggtcttcaaagaaaatcctgatgttgatgccgttattcactttgcggcctattcattggttggtgaatcaatggaaaaaccactcaaatattttgataacaatacagctggaatggttaaattgcttgaagttatgaacgaatgcggtgttaaatacattgtcttctcatcaacagcagcaacttacggaattccagaagaaattccaattcttgaaacaacaccacaaaatcctatcaacccatatggtgaaagtaagttgatgatggaaaccattatgaagtggtctgataaagcttatggtattaaatatgtgccacttcgttacttcaacgttgctggtgcaaaacctgatggatctatcggtgaagaccatggtccagaaactcaccttcttccaattatccttcaagtagctcaaggtgttcgtgaaaaaatcatgatctttggggatgactacaatactccagacgggactaacgttcgtgactatgtgcatccattcgacttggcggacgctcacctccttgctgttgaatatcttcgtaaaggtaatgaatctacagcctttaacttaggttcatcaacaggtttttcaaaccttcaaattcttgaagcagctcgtaaggtaactggtaaagagattccagctgaaaaagttgatcgtcgtcctggtgatcctgatatattgattgcttcatctgaaaaagcacgtacagttcttggatggaaaccacaatttgataacatcgaaaaaatcatcgcaagtgcttgggcatggcattctagccatccaaaagggtacgatgatcgaggataattaaaataataaacaaaaataaagagtatatataattgttgtacttctttgtttttttgaggaaataatatgaaaataagctgcgaaattattggaaaagttgattcaggcgatgtcagtaaaatttcaatggaaaataataatggtgttgtcatttccacactcacaacaggtgctacacttcaggagtttttggttccaacggaaactggtgctcttaaaaatatagtacttggtttcagtgatttcgaggattactataagaataatttatgtgcctgccagtccattggtagggttgctggaagaattgggaaagcttcgtatactcataatatggttctttatagccttcctaagaatgagggagaaaactgcttacatggcggtccaaaaggaatgcaggttcaaaattggaactatgtcacaaacctgaatgatgaatatgttgagacaaaatttattagaagactttattcaagtgtagatggctttcctggtgatgttacagtctctattagttatagacttgacaataataaccgcttaacaatactctttgaagccttcgatgttacagagtcaacagtctttaatccgacaaaccatgtttactttaatcttagcgacaaacaggatttatcaagtcatgagttacaaatctattcagactatcgtttagagttggattctgagttaatccctacaggacaaaaaattaatgtagatggaacaaattatgactttagaaagacaactgacttgctacctcgaattgaagcaaataatggttttgatgatgcctttgtagttgaagggggaacttgtgatcatgtgaaagaagtagctattttgcatgacaaagaaagcggagatggtattgaaatctactcaaacagaaatggtttagtcatttttacgatggatgatatcgaagataatatttactttgcaagagataaaggcaaaatggcaaaagggcgtgaagctattgctctggaagctcaaacccttccagatgccgttaatcacaaaggttttggagatattatcttaaataaaggccatagtgtaaactatgaaattggcttccaatactttaattcatcaagataa

SEQ ID NO: 7 - DNA, 11044 bp, Streptococcus thermophilus
ctattttgtactgtttcttaatgttaattttgttcctagtttagtaagtgttggtatttttcgaggtgctaataattgtttattgagaatatccatagctgttcgtcccatttcttctgtatagacagtgacactagaaagtggaggatacacttgtttagctagagttgtatcgttaaaagagataatttgaatgtcgtcagggaccttgataccattttcctgaagtgctctgagtgcaccaatagctagactatcactagccgcaaagtaagcatcaggtaaagtagctccactcttaatcttagaatcaagaagttcatagccagattggacagtgaagtcaccagtcagaataaaaagagggtcatagattcccttttccatacagtagttccgataagaacgtaaacgaggatcagagatgatttcagttgcatctgttgttttttcttgcccaatgagtaggcctatattattacaaccttgttctttaagataacagagtgccgattgtacggagttttcaaaatcagtagtaacacatggatgtccttgattaagagtatcactatcaacaaagaccagagtcttttttagtctttctagtttcgcaatttgttcacgactaaattttccgatacagagtacaccaacgacctcctctcctagacttgaaggaatgtcgttgaaaaagtgaagcatctcgtagtctagttcataggctcttttttcaataccaagtctaatattataatagtagatgtcatctaactcgtgttcttcactaacccattgaataattgccacttgatacttttctttttttgaattattaattgttttgtatttagtatatccgatttcatcagcgatagttaatatccgatgtctagtatcctctgttacggaaagagtttcatctttattaagaacacgtgaaacagttgaaatagatacacctgctaattttgcgatatctgctaatgtagccatagtatcctcctcatatttcagtataacataacttttatttttttacctatattttactaaaaaaatagtaaaaatattgattttccatgtgaaaggggttacgatttcagtataaacaaaaagaataagtgagatacatcctatgaatacatcacagttaagagaaaagtttaaagaagtttttggtgtagaagcagatcatactttcttttcaccaggtcgtattaatttgattggtgagcatacggactacaatggaggtaacgtccttccggtagctattaccctaggtacttacggagcggcccgcaaacgtgatgacaaagttttacgtttcttctcagctaactttgaagagaagggaatcatcgaagtgccacttgaaaatcttcgttttgaaaatgaacacaactggacaaactatccaaaaggtgttcttcatttcttgcaagaagctgggcatacgattgattcaggtatggatatttacatctatggtaacattccaaacggatcaggcttgtcatcatcatcatctttggaattattgattggtgttattgttgaaaaactttatgacattaaattggaacgcctggacttggttaaaatcggaaaacaaacggaaaatgactttattggcgttaactctggtatcatggaccaattcgctattggtatgggaactgatcaatgtgcgatttacttggacacaaatactctaaagtatgacttggtaccccttgacctcaaggataatgtcgtagtcatcatgaacactaacaaacgtcgtgaattggctgattctaaatacaatgaacgtcgtgctgaatgtgaaacagcagtatctgaactacaagaaaaattggatatccaaactctcggtgaattagacttcttgacatttgacgcatacagctatttgattaaagatgaaaaccgtatcaaacgtgcacgccatgtagttcttgaaaatcaacgtacacttcaagctcgtaaagctcttgaagcaggagatttggaaggctttggacgccttatgaatgcttctcatgtgtcattggaatatgattacgaagttacaggtcttgaacttgatactttggcacacacagcttgggaacaagaaggagtattaggagcccgcatgacaggagctggtttcggtggatgtgccattgcacttgtaaacaaagacaaagttgaagacttcaaaaaagcagttggtcaacgctatgaagaagtcgttggttatgcaccaagcttctatattgccgaagtaactggtggttcacgagtacttgattaatgagaaagaagtagagattatggctgaaaatttagtaaacacttttgtaactcaggttattgaaaatagtgattatgaggaattggaccgtatctatttgacaaacaaagtttttactttggttggagaaggagttgctgatgttgaaacagatagctctgagttgattgaccttaaagaccagttgcttcaagcaggtgttaaagctggttccgtaggtgaacttaacgaagaacaagatatcatcggtgctcaactcatggacttaattacaccaagaccgagcgttgtcaatcgtaacttctgggatacttataaatctaacccagagcaagcaattgctgacttttatgcacaaagtaaacgaaatgattatgtgaaagtcaaagccattgcacaaaatattgcttataaagcaccaactaaatacggtgacttggaaattacgattaacctttcaaaacctgaaaaagatcccaaagccatcgctgcagctaaaaatgcggtagcttctgattatccaaaatgccaactttgtatggaaaatgaaggttatttgggtcgcattaatcacccagcccgcagcaatcaccgtgttgttcgtttccaaatggaagacaaggagtggggcttccaatactcgccttatgcctactttaacgaacattctatcttcttttatggtaagcacgaaccaatgcacatcagtccattgacgtttggccgtctcctaacaattgttgaagcattccctggttacttcgcaggttcaaatgccgatcttccaattgtaggtggttcaattcttacacatgaacactatcaaggtggtcgccataccttcccaatggaagtagcaggcattaaagaaaaagttagctttgatggttactctgatgttgaggctggcatcgttaattggcctatgtctgttcttcgtctaagaagtgaagacaagggaagacttatcgctcttgcaactaaaatcctaaattgctggcgtggttattcagacgaaaaagctggggtcttggctgagtctgatggacaacctcaccacaccattactccaattgctcgtagaaaagacggcaaatttgaattggatttggttcttcgtgacaatcaaacttctgaagaatatccagacggtatctatcacccacataaagatgttcaacatattaagaaagaaaatattggtttgattgaagttatgggattggccattcttccacctcgtttgaaaacagaacttaaagatgttgaagattatctattaggtcaaggtaaccaagttgctccaattcaccaagaatgggcagatgaactcaaagctcaaaatccgaatattacggctgaggaagtgacagaagttgttcgacaatctgttgcagatatctttgctcgtgtactagaagatgcaggtgtttataagactaatagtgaaggcttggatcagtttaaagcatttgtagattttgtaaatttagctgattaattgttttttctgaagaaaggagataaaaaatggcaattttagtattaggtggagctggttatatcggttctcacatggttgatcgcttagttgaaaaaggtcaagaaaaagtagtagtagtagatagtttggtaacaggtcaccgtgctgccgtacatccagatgctattttttatcaaggcgatctttctgatcaggattttatgagaaaggtcttcaaagaaaaccctgatgttgatgctgttattcactttgcggcctattctttggttggtgaatcaatggaaaaaccactcaaatattttgataacaacacagctggaatggttaaattgcttgaggttatgaacgaatgtggtgttaaatacattgtcttctcatcaacagcagcaacttacggaattccagaagaaattccaattcttgaaacaacaccacaaaatcctatcaacccatatggtgaaagtaagttgatgatggaaaccattatgaagtggtcagaccaagcttacggcatcaagtatgtccctcttcgttactttaatgtggcgggtgccaaacctgatggttcgattggtgaggaccacggtccagaaactcaccttctaccgattattcttcaagtagctcaaggtgttcgtgaaaaaatcatgatctttggggatgactacaatactccagacgggactaacgttcgtgactatgtgcatccattcgacttggcggacgctcacctccttgctgttgaatatcttcgtaaaggtaatgaatctacagcctttaacttaggttcatcaacaggtttctcaaaccttcaaattcttgaagcagctcgtaaggtaactggtaaagagattccagctgaaaaagctgatcgtcgtcctggtgatcctgatatattgattgcttcatctgaaaaagcacgtacagttcttggatggaaaccacaatttgataacatcgaaaaaatcatcgcaagtgcttgggcatggcattctagccatccaaaagggtacgatgatcgaggataattaaaataataaacaaaaaaacaaagagtatatataattgttgtactctttgttttttgaggaaataatatgaaaataagctgcgaaattattggaaaagttgattcaggcgatgtcagtaaaatttcaatggaaaataataatggtgttgtcatttccacactcacaacaggtgctacacttcaggagtttttggttccaacggaaactggtgctcttaaaaatatagtacttggtttcagtgatttcgaggattactataagaataatttatgtgcctgccagtccattggtagggttgctggaagaattgggaaagcttcgtatactcataatatggttctttatagccttcctaagaatgagggagaaaactgcttacatggcggtccaaaaggaatgcaggtccaaaattggaactatgtcacaaacctgaatgatgaatatgttgagacaaaatttattagaagactttattcaagtgtagatggctttcctggtgatgtgacagtctctattagttatagacttaacaataataaccgcttaacaatactctttgaagccttcgatgttacagagtcaacaatttttaatccgacaaaccatgtttactttaatcttagcgacaaacaggatttatcaagtcatgagttacaaatctattcagactatcgtttagagttggattctgagttaattccaacaggacaaaaaattaatgtagatgaaacgaattatgattttagaaagacaactgacttgctacctcgaattgaagcaaataatggttttgatgatgcctttgtagttggagggggaacttgtgatcatgtgaaagaagtagctattttgcatgacaaagaaagtggagatggtattgaaatcttctcaaacagaaatggtttagtcatttttacgatggatgatatcgaagataatatttactttgcaagagataaaggcaaaatggcaaaaaggcgtgaagctattgctatggaagctcaaacccttccagatgccgttaatcacaaaggttttggagatattatcttagataaaggtcatagtgtaaactatgaaattggcttccaatactttaattcatcaagataaccatgtattagtaaaattttagtaaaaaacactgaaattattgactgcataaaccaattttcatataatgtaaacgtattcaaataataggaggtttccgaaatggaaaaatctaaaggtcagatgaagtctcgtttatcctacgcagctggtgcttttggtaacgacgtcttctatgcaaccttgtcaacatactttatcatgtttgtgacaactcacttgtttaacacaggtgatccaaagcaaaatagtcactacgtactattaatcactaacattatctctattttgcgtatcttggaagtatttatcgatccattgatcggtaatatgattgataacactaatactaagtatggtaaattcaaaccatgggtagttggtggtggtatcatcagttctatcaccttgttgcttctcttcaccgatttaggtggtttgaataaaacaaatcctttcttgtaccttgtactttttggaattatctaccttataatggatgtcttctactcgattaaagatatcggtttctggtcaatgcttcctgccttgtctcttgacagtcacgaacgtgaaaaaatggcaacttttgcccgtattggttctacgattggtgccaatattgtaggtgttgccatcatgccaatcgttttgttcttctctatgacgaacagtagtggctctggagataaatctggatggttctggtttgcatttatcgttgcactcattggtgtgattacatcaattgctgttggtattggtacacgtgaagttgagtcaaaacttcgtgataacaatgaaaaaactagccttaaacaagtctttaaagttcttggtaaaaacgaccaattgatgtggctgtctctcggatattggttctatggtcttggtattaatacacttaatgctcttcaactttattacttcacatttatccttggtgattcagggaaatactcaatcctttacggattgaatacagttgttggtttggtttcagtttcactgttccctagtcttgctggtaaattcaaccgtaaacgtttgttctatggatgtattgcagtaatgctcggtggtatcggaatctttagtattgcaggtacttcacttccaatgatcttgactgcagctgaactcttcttcattccacaacctcttgtgttccttgttatccttatgattatctctgactcagtagaatatggtcaatggaaattgggacaccgtgatgaatcacttactttgtcagttcgtccacttgttgataaacttggtggtgcgatgtcaaactggcttgtttctacaattgccgtagctgccggtatgacaacaggtgcctcagcatcaacaattacaacacatcaacagtctatttttaagcttagcatgtttggtttcccagcagcagcaatgcttatcggtgccttcattattgctcgtaaaatcactttgactgaagcacgtcacgctaaaattgttgaagaattggaacatcgctttagcgtagcaacttctgaaaatgaagttaaagctaacgtcgtatctcttgtaacccctacaactggttatttggttgatctctcaagtgttaatgatgaacactttgcttcaggtagcatgggtaaaggtttcgccattaaacctactgatggagctgtctttgcaccaattagtggtaccattcgtcaaattcttcctactcgccatgcagttggtattgaaagtgaagatggtgtcattgttcttatccacgttggcatcggaacagttaaacttaatggtgaaggattcattagttacgtagaacaaggtgatcgtgttgaagttggacaaaaacttcttgagttctggtcaccaattattgagaaaaatggtcttgatgacacagtacttgtcactgtaactaattcagaaaaattcagtgctttccatcttgaacaaaaagttggagaaaaggtagaagctttgtctgaagttattaccttcaaaaaaggagaataatctatgaacatgactgaaaaaattcaaacttatttaaacgatccaaagattgttagcgttaatactgttgatgctcactcagatcataagtattttgaatctcttgaagaattttctgaaggggagatgaagttaagacaatctcttaatggaaaatggaaaattcactatgctcagaatacaaatcaggttttaaaagacttttataaaacagaatttgatgaaactgatttgaatttcatcaatgtaccaggtcatttagagcttcaaggttttggttctccacaatatgtgaatacccaatatccttgggatggtaaagaattccttcgtccacctcaagttcctcaagaatcaaatgctgttgcatcatacgttaaacattttaccttgaatgatgcattaaaagataaaaaagtatttatctcattccaaggggttgctacttccatctttgtatgggtcaatggtaacttcgtaggatacagtgaagattcatttacacctagtgaatttgaaattagtgattaccttgttgaaggtgataacaagttggcggtagctgtttatcgttactctacagcaagctggttggaagaccaagacttctggagactttacggtatttttagagatgtttacttgtatgctattccaaaagttcacgttcaagatctctttgttaagggagattatgattaccaaacaaaagcaggtcaattggatattgatttgaagactgttggtgattatgaagacaagaagattaaatatgttctttcagattatgaaggcatcgttacagaaggtgatgcatctgttaatggtgacggtgaactatctgtaagtcttgaaaatcttaaaatcaaaccttggagtgctgaaagtcctaaactttacgatttgatccttcatgttttggatgatgaccaagttgttgaagtcgttccagttaaagttggattcagacgctttgaaattaaagataaacttatgcttttgaatggtaagagaattgtctttaaaggggttaacagacacgaatttaacgctagaacaggacgttgtatcactgaagaagatatgctttgggatatcaaagtgatgaagcaacataacatcaatgctgttcgtacttcacactatcctaaccaaacacgttggtatgaattgtgtgatgaatatggactttatgttatcgatgaagccaaccttgaaacacacggtacatggcaaaaacttggtctatgcgaaccttcatggaatatcccagctagtgaaccagaatggttgcctgcttgtttggatcgtgccaataacatgttccaacgcgataagaaccacgctagtgttatcatttggtcttgtggtaatgaatcatatgctggtaaagatattgctgacatggctgattacttccgtagtgttgacaatactcgtccagttcactatgaaggtgttgcatggtgtcgtgagtttgattacattacagacatcgaaagtcgtatgtatgcgaaaccagctgatatcgaagaatacctcacaactggtaaactagttgatctttcaagcgttagtgataaacactttgcttcaggtaacctaactaacaaacctcaaaaaccttatatttcatgtgaatacatgcacacaatgggtaactctggtggtggattgcaactctacactgacttagagaaatatccagaataccaaggtggatttatttgggacttcattgaccaagctatttacaaaacacttccaaatggtagcgaattcctatcatatggtggtgactggcatgatagaccttctgactacgaattttgtggaaatggtatcgtctttgcagatcgtaccctaactccaaaacttcaaacagttaaacatctttactctaatattaagattgctgttgatgaaaaatcagtaactatcaagaatgataatctcttcgaagatctttctgcttatactttcctagctagagtttacgaagatggtagaaaagttagtgaaagtgaatatcactttgatgttaaaccaggcgaagaagcaacattcccagttaactttgtagtcgaggcttcaaattctgaacaaatttacgaagttgcttgtgttctgagggaagcaactgaatgggctcctaaaggtcatgaaattgttcgtggtcaatatgttgttgaaaagattagcactgaaacaccagttaaagcacctttgaatgttgttgaaggcgacttcaacatcggtattcaaggacaaaacttctcaatcttgctttcacgtgcacaaaatactttagtatctgctaagtataatggtgttgaattcattgagaaaggtcctaaacttagcttcactcgtgcttacactgacaacgatcgtggtgctggatatccattcgaaatggcaggctggaaggttgctggaaactatagtaaagttacagatactcaaattcaaatcgaagacgactctgttaaagtgacttatgttcatgaattgccaggcttgtctgatgtcgaagttaaggtaacttatcaagttgattacaagggtcgaatctttgttactgcaaactatgatggtaaagcaggtttgccaaacttccctgaatttggtctagaatttgctatcggttcacaatttacaaaccttagctattatggatacggtgcagaagaaagctaccgtgataaacttcctggtgcctatcttggtcgatatgaaacatctgttgaaaagacatttgctccatatctaatgccacaagaatctggtaatcactatggtactcgtgaattcacagtatctgatgataaccataatggtcttaaattcaccgcacttaataaagcattcgaattcagtgctttgcgtaacagtactgaacaaattgaaaatgctcgtcaccaatatgagttgcaagaatctgatgctacatggattaaagttcttgctgctcaaatgggtgtaggtggtgacgacacatggggtgctccagttcatgacgaattcttgcttagctcagcagatagctatcaattaagcttcatgattgaaccactaaattag

SEQ ID NO: 8 - DNA, 11044 bp, Streptococcus thermophilus
ctattttgtactgtttcttaatgttaattttgttcctagtttagtaagtgttggtatttttcgaggtgctaataattgtttattgagaatatccatagctgttcgtcccatttcttctgtatagacagtgacactagaaagtggaggatacacttgtttagctagagttgtatcgttaaaagagataatttgaatgtcgtcagggaccttgataccattttcctgaagtgctctgagtgcaccaatagctagactatcactagccgcaaagtaagcatcaggtaaagtagctccactcttaatcttagaatcaagaagttcatagccagattggacagtgaagtcaccagtcagaataaaaagagggtcatagattcccttttccatacagtagtttcgataagaacgtaaacgaggatcagaaatgatttcagttgcatctgttgttttttcttgcccaatgagtaggcctatattattacaaccttgttctttaagataacagagtgccgattgtacggagttttcaaaatcagtagtaacacatggatgtccttgattaagagtatcactatcaacaaagaccagagtcttttttagtctttctagtttcgcaatttgttcacgactaaattttccgatacatagtacaccaacgacctcctctcctagacttgaaggaatgtcgttgaaaaagtgaagcatctcgtagtctagttcataggctcttttttcaataccaagtctaatattataatagtagatgtcatctaactcgtgttcttcactaacccattgaataattgccacttgatacttttctttttttgaattattaattgttttgtatttagtatatccgatttcatcagcgatagttaatatccgatgtctagtatcctctgttacggaaagagtttcatctttattaagaacacgtgaaacagttgaaatagatacacctgctaattttgcgatatctgctaatgtagccacagtatcctcctcatatttcagtataacataacttttatttttttacctatattttactaaaaaaatagtaaaaatattgattttccatgtgaaaggggttacgatttcagtataaacaaaaagaataagtgagatacatcctatgaatacatcacagttaagagaaaagtttaaagaagtttttggtgtagaagcagatcatactttcttttcaccaggtcgtattaatttgattggtgagcatacggactacaatggaggtaacgtccttccggtagctattaccctaggtacttacggagcggcccgcaaacgtgatgacaaagttttgcgtttcttctcagctaactttgaagagaagggaatcatcgaagtgccacttgaaaatcttcgttttgaaaaagaacacaactggacaaactatccaaaaggtgttcttcatttcttgcaagaagctgggcatacgattgattcaggtatggatatttacatctatggtaacattccaaacggatcaggcttgtcatcatcatcatctttggaattattgattggtgttattgttgaaaaactttatgacattaaattggaacgcctggacttggttaaaatcggaaaacaaacggaaaatgactttattggcgttaactctggtatcatggaccaattcgctattggtatgggagctgatcaatgtgcgatttacttggacacaaatactctaaagtatgacttggtaccccttgacctcaaggataatgtcgtagtcatcatgaacactaacaaacgtcgtgaattggctgattctaaatacaatgaacgtcgtgctgaatgtgaaacagcagtatctgaactacaagaaaaattggatatccaaactctcggtgaattagacttcttgacatttgatgcatacagctatttgattaaagatgaaaaccgtatcaaacgtgcacgccatgtagttcttgaaaatcaacgtacacttcaagctcgtaaagctcttgaagcaggagatttggaaggctttggacgccttatgaatgcttctcatgtgtcattggaatatgattacgaagttacaggtcttgaacttgatactttggcacacacagcttgggaacaagaaggagtattaggagcccgcatgacaggagctggtttcggtggatgtgccattgcacttgtaaacaaagacaaagttgaagacttcaaaaaagcagttggtcaacgctatgaagaagtcgttggttatgcaccaagcttctatattgccgaagtaactggtggttcacgagtacttgattaatgagaaagaagtagagattatggctgaaaatttagtaaacacttttgtaactcaggttattgaaaatagtgattatgaggaattggaccgtatctatttgacaaacaaagtttttactttggttggagaaggagttgctgatgttgaaacagatagctctgaattgattgaccttaaagaccagttgcttcaagcaggtgttaaagctggttccgtaggtgaacttaacgaagaacaagatatcatcggtgctcaactcatggacttaattacaccaagaccgagcgttgtcaatcgtaacttctgggatacttataaatctaacccagagcaagcaattgctgacttttatgcacaaagtaaacgaaatgattatgtgaaagtcaaagccattgcacaaaatattgcttataaagcaccaactaaatacggtgacttggaaattacgattaacctttcaaaacctgaaaaagatcccaaagccatcgctgcagctaaaaatgcggtagcttctgattatccaaaatgccaactttgtatggaaaatgaaggttatttgggtcgcattaatcacccagcccgcagcaatcaccgtgttgttcgtttccaaatggaagacaaggagtggggcttccaatactcgccttatgcctactttaacgaacattctatcttcttttatggtaagcacgaaccaatgcacatcagtccattgacgtttggccgtctcctaacaattgttgaagcattccctggttacttcgcaggttcaaatgccgatcttccaattgtaggtggttcaattcttacacatgaacactatcaaggtggtcgccataccttcccaatggaagtagcaggcattaaagaaaaagttagctttgatggttactctgatgttgaggctggcatcgttaattggcctatgtctgttcttcgtctaagaagtgaagacaaggaaagacttatcgctcttgcaactaaaatcctaaattgctggcgtggttattcagacgaaaaagctggggtcttggctgagtctgatggacaaccacaccacaccattactccaattgctcgtagaaaagacggcaaatttgaattggatttggttcttcgtgacaatcaaacttctgaagaatatccagacggtatctatcacccacataaagatgttcaacatattaagaaagaaaatattggtttgattgaagttatgggattggccattcttccacctcgtttgaaaacagaacttaaagatgttgaagattatctattaggtcaaggtaaccaagttgctccaattcaccaagaatgggcagatgaactcaaagctcaaaatccaaatattacggctgaggaagtgacagaagttgttcgacaatctgttgcagatatctttgctcgtgtactagaagatgcaggtgtttataagactaatagtgaaggcttggatcagtttaaagcatttgtagattttgtaaatttagctgattaattgttttttctgaagaaaggagataagaaatggcaattttagtattaggtggagctggttatatcggttctcacatggttgatcgcttagttgaaaaaggtcaagaaaaagtagtagtagtagatagtttggtaacaggtcaccgtgctgccgtacatccagatgctattttctatcaaggcgatctttctgatcaggattttatgagaaaggtcttcaaagaaaaccctgatgttgatgctgttattcactttgcggcctattctttggttggtgaatcaatggaaaaaccactcaaatattttgataacaacacagctggaatggttaaattgcttgaggttatgaacgaatgcggtgttaaatacattgtcttctcatcaacagcagcaacttacggaattccagaagaaattccaattcttgaaacaacaccacaaaatcctatcaacccatatggtgaaagtaagttgatgatggaaaccattatgaagtggtcagaccaagcttacggcatcaagtatgtccctcttcgttactttaatgtggcgggtgccaaacctgatggttcgattggtgaggaccacgatccagaaactcaccttctaccgattattcttcaagtagctcaaggtgttcgtgaaaaaatcatgatctttggggatgactacaatactccagacgggactaacgttcgtgactatgtgcatccattcgacttggcggacgctcacctccttgctgttgaatatcttcgtaaaggtaatgaatctacagcctttaacttaggttcatcaacaggtttctcaaaccttcaaattcttgaagcagctcgtaaggtaactggtaaagagattccagctgaaaaagctgatcgtcgtcctggtgatcctgatatattgattgcttcatctgaaaaagcacgtacagttcttggatggaaaccacaatttgataacatcgaaaaaatcatcgcaagtgcttgggcatggcattctagccatccaaaagggtacgatgatcgaggataattaaaataataaacaaaaaaacaaagagtatatataattgttgtactctttgttttttgaggaaataatatgaaaataagctgcgaaattattggaaaagttgattcaggcgatgtcagtaaaatttcaatggaaaataataatggtgttgtcatttccacactcacaacaggtgctacacttcaggagtttttggttccaacggaaactggtgctcttaaaaatatagtacttggtttcagtgatttcgaggattactataagaataatttatgtgcctgccagtccattggtagggttgctggaagaattgggaaagcttcgtatactcataatatggttctttatagccttcctaagaatgagggagaaaactgcttacatggcggtccaaaaggaatgcaggtccaaaattggaactatgtcacaaacctgaatgatgaatatgttgagacaaaatttattagaagactttattcaagtgtagatggctttcctggtgatgtgacagtctctattagttatagacttaacaataataaccgcttaacaatactctttgaagccttcgatgttacagagtcaacagtctttaatccgacaaaccatgtttactttaatcttagcgacaaacaggatttatcaagtcatgagttacaaatctattcagactatcgtttagagttggattctgagttaattccaacaggacaaaaaattaatgtagatgaaacgaattatgattttagaaagacaactgacttgctacctcgaattgaagcaaataatggttttgatgatgcctttgtagttggagggggaacttgtgatcatgtgaaagaagtagctattttgcatgacaaagaaagcggagatggtattgaaatcttctcaaacagaaatggtttagtcatttttacgatggatgatatcgaagataatatttactttgcaagagataaaggcaaaatggcaaaaaggcgtgaagctattgctatggaagctcaaacccttccagatgccgttaatcacaaaggttttggagatattatcttagataaaggtcatagtgtaaactatgaaattggcttccaatactttaattcatcaagataaccatgtattagtaaaattttagtaaaaaacactgaaattattgactgcataaaccaattttcatataatgtaaacgtattcaaataataggaggtttccgaaatggaaaaatctaaaggtcagatgaagtctcgtttatcctacgcagctggtgcttttggtaacgacgtcttctatgcaaccttgtcaacatactttatcatgtttgtgacaactcacttgtttaacacaggtgatccaaagcaaaatagtcactacgtactattaatcactaacattatctctattttgcgtatcttggaagtatttatcgatccattgatcggtaatatgattgataacactaatactaagtatggtaaattcaaaccatgggtagttggtggtggtatcatcagttctatcaccttgttgcttctcttcaccgatttaggtggtttgaataaaacaaatcctttcttgtaccttgtactttttggaattatctaccttgtaatggatgtcttctactcgattaaagatatcggtttctggtcaatgattcctgccttgtctcttgatagtcacgaacgtgaaaaaatggcaacttttgcccgtattggttctacgattggtgccaatattgtaggtgttgccatcatgccaatcgttttgttcttctctatgacgaacaatagtggctctggagataaatctggatggttctggtttgcatttatcgttgctctcattggtgtgattacatcaattgctgttggtattggtacacgtgaagttgagtcaaaaattcgtgataataacgaaaaaactagccttaaacaagtctttaaggttcttggtcaaaacgaccaattgatgtggttatctcttggatattggttctatggtcttggtattaatacacttaatgctcttcaactttattatttcacatttatccttggtgattcaggtaaatactcaattctttacggattgaatacagttgttggtttggtttcagtttcactcttccctaccctagctgataaattcaaccgtaaacgtttgttctacggatgtattgcagtaatgctcgggggtatcggaatatttagtattgcaggtacatcacttccaataatcttgactgcagctgaactcttcttcattccacaacctcttgtgttccttgttgtctttatgattatctctgactcagtagaatatggtcaatggaaaacgggacaccgtgatgaatcacttactttgtcagttcgtccacttattgataaacttggtggtgcgatgtcaaactggcttgtttctacatttgccgtagctgccggtatgacaacaggtgcctcagcatcaacaattacaacacatcaacagtttatctttaagcttggcatgtttgctttcccagcagcaacaatgcttatcggtgccttcattgttgctcgtaaaatcactttgactgaagcacgtcacgctaaaattgttgaagaattggaacatcgctttagcgtagcaacttctgaaaatgaagttaaagctaacgtcgtatctcttgtaacccctacaactggttatttggttgatctctcaagtgttaatgatgaacactttgcttcaggtagcatgggtaaaggtttcgccattaaacctactgatggagctgtctttgcaccaattagtggtaccattcgtcaaattcttcctactcgccatgcagttggtattgaaagtgaagatggtgtcattgttcttatccacgttggcatcggaacagttaaacttaatggtgaaggattcattagttacgtagaacaaggtgatcatgttgaagttggacaaaaacttcttgagttctggtcaccaattattgagaaaaatggtcttgatgacacagtacttgtcactgtaactaattcagaaaaattcagtgctttccatcttgaacaaaaagttggagaaaaggtagaagctttgtctgaagttattaccttcaaaaaaggagaataatctatgaacatgactgaaaaaattcaaacttatttaaacgatccaaagattgttagcgttaatactgttgatgctcactcagatcataagtattttgaatctcttgaagaattttctgaaggggagatgaagttaagacaatctcttaatggaaaatggaaaattcactatgctcagaatacaaatcaggttttaaaagacttttataaaacagaatttgatgaaactgatttgaatttcatcaatgtaccaggtcatttagagcttcaaggttttggttctccacaatatgtgaatacccaatatccttgggatggtaaagaattccttcgtccacctcaagttcctcaagaatcaaatgctgttgcatcatacgttaaacattttaccttgaatgatgcattaaaagataaaaaagtatttatctcattccaaggggttgctacttccatctttgtatgggtcaatggtaacttcgtaggatacagtgaagattcatttacacctagtgaatttgaaattagtgattaccttgttgaaggtgataacaagttggcggtagctgtttatcgttactctacagcaagctggttggaagaccaagacttctggagactttacggtatttttagagatgtttacttgtatgctattccaaaagttcacgttcaagatctctttgttaagggagattatgattaccaaacaaaagcaggtcaattggatattgatttgaagactgttggtgattatgaagacaagaagattaaatatgttctttcagattatgaaggcatcgttacagaaggtgatgcatctgttaatggtgacggtgaactatctgtaagtcttgaaaatcttaaaatcaaaccttggagtgctgaaagtcctaaactttacgatttgatccttcatgttttggatgatgaccaagttgttgaagtcgttccagttaaagttggatttagacgctttgaaattaaagataaacttatgcttttgaatggtaagagaattgtctttaaaggggttaacagacacgaatttaacgctagaacaggacgttgtatcactgaagaagatatgctttgggatatcaaagtgatgaaacaacataacatcaatgctgttcgtacttcacactatcctaaccaaacacgttggtatgaattgtgtgatgaatatggactttatgttatcgatgaagccaaccttgaaacacacggtacatggcaaaaacttggtctatgcgaaccttcatggaatatcccagctagtgaaccagaatggttgcctgcttgtttggatcgtgccaataacatgttccaacgcgataagaaccacgctagtgttatcatttggtcttgtggtaatgaatcatatgctggtaaagatattgctgacatggctgattacttccgtagtgttgacaatactcgtccagttcactatgaaggtgttgcatggtgtcgtgagtttgattacattacagacatcgaaagtcgtatgtatgcgaaaccagctgatatcgaagaatacctcacaactggtaaactagttgatctttcaagcgttagtgataaacactttgcttcaggtaacctaactaacaaacctcaaaaaccttatatttcatgtgaatacatgcacacaatgggtaactctggtggtggattgcaactctacactgacttagagaaatatccagaataccaaggtggatttatttgggacttcattgaccaagctatttacaaaacacttccaaatggtagcgaattcctatcatatggtggtgactggcatgatagaccttctgactacgaattttgtggaaatggtatcgtctttgcagatcgtaccctaactccaaaacttcaaacagttaaacatctttactctaatattaagattgctgttgatgaaaaatcagtaactatcaagaatgataatctcttcgaagatctttctgcttatactttcctagctagagtttacgaagatggtagaaaagttagtgaaagtgaatatcactttgatgttaaaccaggcgaagaagcaacattcccagttaactttgtagtcgaggcttcaaattctgaacaaatttacgaagttgcttgtgttctgagggaagcaactgaatgggctcctaaaggtcatgaaattgttcgtggtcaatatgttgttgaaaagattagcactgaaacaccagttaaagcacctttgaatgttgttgaaggcgacttcaacatcggtattcaaggacaaaacttctcaatcttgctttcacgtgcacaaaatactttagtatctgctaagtataatggtgttgaattcattgagaaaggtcctaaacttagcttcactcgtgcttacactgacaacgatcgtggtgctggatatccattcgaaatggcaggctggaaggttgctggaaactatagtaaagttacagatactcaaattcaaatcgaagacgactctgttaaagtgacttatgttcatgaattgccaggcttgtctgatgtcgaagttaaggtaacttatcaagttgattacaagggtcgaatctttgttactgcaaactatgatggtaaagcaggtttgccaaacttccctgaatttggtctagaatttgctatcggttcacaatttacaaaccttagctattatggatacggtgcagaagaaagctaccgtgataaacttcctggtgcctatcttggtcgatatgaaacatctgttgaaaagacatttgctccatatctaatgccacaagaatctggtaatcactatggtactcgtgaattcacagtatctgatgataaccataatggtcttaaattcaccgcacttaataaagcattcgaattcagtgctttgcgtaacagtactgaacaaattgaaaatgctcgtcaccaatatgagttgcaagaatctgatgctacatggattaaagttcttgctgctcaaatgggtgtaggtggtgacgacacatggggtgctccagttcatgacgaattcttgcttagctcagcagatagctatcaattaagcttcatgattgaaccactaaattag

SEQ ID NO: 9 - DNA, 19 bp, Artificial Sequence
  Oligonucleotide primer
tgttcgtgctgacttgcac

SEQ ID NO: 10 - DNA, 19 bp, Artificial Sequence
  Oligonucleotide primer
cctcgaggtcgacggtatc

SEQ ID NO: 11 - DNA, 20 bp, Artificial Sequence
  Oligonucleotide primer
atcgtccagacaatggcatg

SEQ ID NO: 12 - DNA, 41 bp, Artificial Sequence
  Oligonucleotide primer
gtgcaagtcagcacgaacactgaaccataaacctgaatagg

SEQ ID NO: 13 - DNA, 39 bp, Artificial Sequence
  Oligonucleotide primer
gataccgtcgacctcgagggtactgattagcactccaac

SEQ ID NO: 14 - DNA, 20 bp, Artificial Sequence
  Oligonucleotide primer
agattaccctgcctcaattg

SEQ ID NO: 15 - DNA, 20 bp, Artificial Sequence
  Oligonucleotide primer
cagtagttccgataagaacg

SEQ ID NO: 16 - DNA, 20 bp, Artificial Sequence
  Oligonucleotide primer
gtttcacattcagcacgacg

SEQ ID NO: 17 - DNA, 20 bp, Artificial Sequence
  Oligonucleotide primer
aattgccacttgatactttt